(19) United States
(12) Patent Application Publication
(10) Pub. No.: US 2011/0224231 A1
(43) Pub. Date: Sep. 15, 2011

(54) Novel Lactams as Beta Secretase Inhibitors

(75) Inventors: Michael A. Brodney, East Lyme, CT (US); Ivan V. Efremov, Gales Ferry, CT (US); Christopher J. Helal, Mystic, CT (US); Brian T. O'Neill, Haddam, CT (US)

(73) Assignee: Pfizer Inc., Groton, CT (US)

(21) Appl. No.: 13/130,192

(22) PCT Filed: Nov. 12, 2009
(86) PCT No.: PCT/IB2009/055043

§ 371 Date: May 19, 2011

Related U.S. Application Data
(60) Provisional application No. 61/117,225, filed on Nov. 23, 2008.

(51) Int. Cl. A61K 31/4965; C07D 471/10; A61K 31/438; A61P 25/00; A61P 25/18

(52) U.S. Cl. 514/255.05; 544/230; 546/16; 514/278

(57) ABSTRACT

Compounds and pharmaceutically acceptable salts of the compounds are disclosed, wherein the compounds have the structure of Formula (I) as defined in the specification. Corresponding pharmaceutical compositions, methods of treatment methods of synthesis, and intermediates are also disclosed.

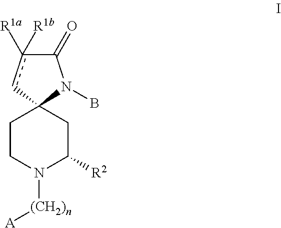

### FIELD OF THE INVENTION

The present invention relates to the treatment of Alzheimer's disease and other neurodegenerative and/or neurological disorders in mammals, including humans. This invention also relates to inhibiting, in mammals, including humans, the production of A˜beta peptides that can contribute to the formation of neurological deposits of amyloid protein. More particularly, this invention relates to spiro˜piperidine compounds useful for the treatment of neurodegenerative and/or neurological disorders, such as Alzheimer's disease and Down's Syndrome, related to A-beta peptide production.

### BACKGROUND OF THE INVENTION

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner et al., J. Neural. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Several programs have been advanced by research groups to ameliorate the pathological processes causing dementia, AD, CM and prion-mediated diseases. Beta-secretase (BACE) inhibitors are one such strategy and numerous compounds are under evaluation by pharmaceutical groups. The present invention relates to a group of brain˜penetrable BACE inhibitors and as such would be expected to be BACE inhibitors and modulators for the treatment of AD (see Ann. Rep. Med. Chem. 2007, Olsen et al., 42: 27-47).

### SUMMARY OF THE INVENTION

The invention is directed to a compound, including the pharmaceutically acceptable salts thereof, having the structure of formula I:

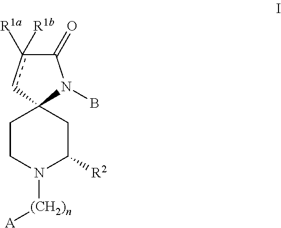

wherein the stereochemistry shown in formula I at the carbon bonded to R² and at the spirocyclic carbon is the absolute stereochemistry; B is alkyl, aryl, heteroaryl, cycloalkyl, or heterocycloalkyl, wherein B is optionally substituted with zero to three R³ groups:

A is independently aryl, cycloalkyl, heterocycloalkyl or heteroaryl wherein said aryl, cycloalkyl, heterocycloalkyl or heteroaryl is optionally substituted with one to three R⁴;

when [symbol] is a single bond, R^{1a} and R^{1b} are each independently hydrogen, alkyl, alkenyl, —(CH₂)ₜ-cycloalkyl, —(CH₂)ₜ-heterocycloalkyl, —(CH₂)ₜ-aryl, —(CH₂)ₜ-heteroaryl, ˜(CH₂)ₜ˜OR⁵, ˜(CH₂)ₜN(R⁷)₂, ˜NH˜(CH₂)ₜ˜cycloalkyl, ˜NH˜(CH₂)ₜ˜heterocycloalkyl, —NH—(CH₂)ₜ-aryl, —NH—(CH₂)ₜ-heteroaryl, —(CH₂)ₜ—COR⁵, —(CH₂)ₜ—SO₂R⁵, or —(CH₂)ₜ—CO₂R⁵; wherein said alkyl, alkenyl, —(CH₂)ₜ-cycloalkyl, —(CH₂)ₜ-heterocycloalkyl, —(CH₂)ₜ-aryl, or —(CH₂)ₜ-heteroaryl R^{1a} or R^{1b} substituent is optionally substituted with one to three hydroxyl, aryl, heteroaryl, halogen, alkyl, cycloalkyl, ˜SO₂R⁷, ˜NR⁷COR⁷, ˜CON(R⁷)₂, ˜COOR⁷, ˜C(O)R⁷, ˜CN, or ˜N(R⁷)₂ wherein said aryl, alkyl, cycloalkyl and heteroaryl substituent is optionally substituted with one to three halogen, alkyl, hydroxyl, or ˜O˜alkyl; or R^{1a} and R^{1b} together with the carbon they are bonded to form a cycloalkylene moiety or a heterocycloalkylene moiety, wherein said cycloalkylene or heterocycloalkylene moiety is optionally substituted with one to three hydroxyl, aryl, heteroaryl, halogen, alkyl, cycloalkyl, ˜SO₂R⁷, ˜NR⁷, ˜COR⁷, ˜CON(R⁷)₂, —COOR⁷, —C(O)R⁷, —CN, or —N(R⁷)₂, wherein said aryl, alkyl, cycloalkyl and heteroaryl substituent is optionally substituted with one to three halogen, alkyl, hydroxyl, or —O-alkyl;

when [symbol] is a double bond, R^{1b} is absent and R^{1a} is hydrogen, alkyl, alkenyl, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, ˜(CH₂)ₜ˜OR⁵, ˜(CH₂)ₜN(R⁷)₂, ˜NH˜(CH₂)ₜ˜cycloalkyl, ˜NH˜(CH₂)ₜ˜heterocycloalkyl, ˜NH˜(CH₂)ₜ-aryl, —NH—(CH₂)ₜ-heteroaryl, —(CH₂)ₜ—COR⁵, —(CH₂)ₜ—SO₂R⁵, or —(CH₂)ₜ—CO₂R⁵, wherein said alkyl, alkenyl, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl R^{1a} substituent is optionally substituted with one to three hydroxyl, aryl, heteroaryl, halogen, alkyl, cycloalkyl, —SO₂R⁷, —NR⁷, —COR⁷, —CON(R⁷)₂, —COOR⁷, —C(O)R⁷, or —N(R⁷)₂, wherein said aryl, alkyl, cycloalkyl and heteroaryl substituent is optionally substituted with one to three halogen, alkyl, hydroxyl, or ˜O˜alkyl;

R² is alkyl, cycloalkyl, or alkenyl wherein said alkyl, cycloalkyl, or alkenyl is optionally substituted with one to three halogen, hydroxyl, or cyano;

each R³ is independently halogen, alkyl, cyano, hydroxyl, —O-alkyl, —O-cycloalkyl, ˜SO₂R⁷, ˜N(R⁷)₂, ˜COR⁷, ˜CON(R⁷)₂, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl wherein said R³ alkyl, ˜(CH₂)ₜ˜cycloalkyl, —(CH₂)ₜ-heterocycloalkyl, —(CH₂)ₜ-aryl, or —(CH₂)ₜ-heteroaryl is optionally substituted with one to three R⁴;

each R⁴ is independently alkyl, halogen, cyano, —SO₂NHR⁷, —CON(R⁷)₂, ˜N(R⁷)₂, ˜N(R⁷)COR⁷, ˜N(R⁷)CO₂R⁷, ˜SO₂N(R⁷)₂, ˜N(R⁷)SO₂R⁷, ˜COR⁷, ˜SO₂R⁷, —(CH₂)ₜ-cycloalkyl, —(CH₂)ₜ-heterocycloalkyl, —(CH₂)ₜ-aryl, —(CH₂)ₜ-heteroaryl, —(CH₂)ₜ—N(R⁷)₂, or —(CH₂)ₜ—OR⁵; wherein each R¹ alkyl, —(CH₂)ₜ-cycloalkyl, —(CH₂)ₜ-heterocycloalkyl, —(CH₂)ₜ-aryl, or —(CH₂)ₜ-heteroaryl is optionally independently substituted by one to three cyano, alkyl, halogen, ˜CF₃ or ˜OR⁵;

each R⁵ is independently hydrogen, alkyl, ˜(CH₂)ₜ˜cycloalkyl, ˜(CH₂)ₜ˜heterocycloalkyl, —(CH₂)ₜ-aryl, or —(CH₂)ₜ-heteroaryl; wherein said —(CH₂)ₜ-cycloalkyl, ˜(CH₂)ₜ˜heterocycloalkyl, ˜(CH₂)ₜ˜aryl, or ˜(CH₂)ₜ˜heteroaryl is optionally substituted with one to three R⁶;

each R⁶ is independently alkyl, hydroxyl, alkoxy, halogen, cyano, ˜(CH₂)ₜN(R⁷)₂, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl;

each R⁷ is independently hydrogen, alkyl, —(CH₂)ₜ-cycloalkyl, —(CH₂)ₜ-heterocycloalkyl, —(CH₂)ₜ-aryl, or —(CH₂)ₜ-heteroaryl, or when two R⁷ substituents are attached to the same nitrogen atom they may be taken together with the nitrogen to which they are attached to form a heterocycloalkylene moiety; and wherein said alkyl, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl are optionally substituted with one to three alkyl, halogen, cyano, hydroxyl, or —OR⁴;

n is an integer selected from 1, 2 and 3; and

each t is an integer independently selected from 0, 2 and or pharmaceutically acceptable salts thereof.

In another embodiment of the invention, n=1.

In a further embodiment of the invention [symbol] is a single bond, and R^{1a} and R^{1b} are each independently hydrogen or alkyl. In one example of this embodiment, R^{1a} and R^{1b} together with the carbon they are bonded to form a cycloalkylene moiety or a heterocycloalkylene moiety. In another example of this embodiment, R^{1a} and R^{1b} together with the carbon they are bonded to form a cycloalkylene moiety or a heterocycloalkylene moiety. In another example of this embodiment, R^{1a} and R^{1b} are each hydrogen.

In another embodiment of the invention [symbol] is a double bond, and R^{1b} is absent.

In another embodiment of the invention, A is aryl.

In another embodiment of the invention, A is cycloalkyl.

In another embodiment of the invention, A is heteroaryl.

In another embodiment of the invention, A is heterocycloalkyl.

In another embodiment of the invention, A is aryl, heteroaryl, cycloalkyl or heterocycloalkyl, and A is optionally substituted with one R⁴ substituent. In one example of this embodiment, R⁴ is independently alkyl, halogen, cyano, —SO₂NHR⁷, ˜CON(R⁷)₂, ˜N(R⁷)₂, ˜N(R⁷)COR⁷, ˜SO₂N(R⁷)₂, ˜N(R⁷)SO₂R⁷, ˜COR⁷, ˜SO₂R⁷, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, ˜(CH₂)ₜ˜N(R⁷)₂, or —(CH₂)ₜ—OR⁵ wherein each R⁴ alkyl, —(CH₂)ₜ-cycloalkyl, —(CH₂)ₜ-heterocycloalkyl, —(CH₂)ₜ-aryl, or —(CH₂)ₜ-heteroaryl is optionally independently substituted by one to three cyano, alkyl, halogen, ˜CF₃, or ˜OR⁵. In one example of this embodiment, A is aryl or heteroaryl, and R⁴ is independently alkyl, halogen, cyano, —SO₂NHR⁷, —CON(R⁷)₂, —N(R⁷)₂, —N(R⁷)COR⁷, —SO₂N(R⁷)₂, —N(R⁷)SO₂R⁷, —SO₂R⁷, —(CH₂)ₜ-cycloalkyl, —(CH₂)ₜ-heterocycloalkyl, —(CH₂)ₜ-aryl, —(CH₂)ₜ-heteroaryl, —(CH₂)ₜ—N(R⁷)₂, or ˜(CH₂)ₜ˜OR⁵ wherein each R⁴ alkyl, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl is optionally independently substituted by one to three cyano, alkyl, halogen, ˜CF₃, or ˜OR⁵. In another example of this embodiment, R⁴ is halogen, alkyl, —OR⁵, cyano, trifluoroalkyl, —(CH₂)ₜ-cycloalkyl, —(CH₂)ₜ-heterocycloalkyl, ˜(CH₂)ₜ˜aryl, or ˜(CH₂)ₜ˜heteroaryl, wherein each R⁴ ˜(CH₂)ₜ˜cycloalkyl, ˜(CH₂)ₜ˜heterocycloalkyl, ˜(CH₂)ₜ˜aryl, or ˜(CH₂)ₜ˜heteroaryl, is optionally independently substituted by one to three —OR⁵, alkyl, cyano, or halogen. In an example of this embodiment, A is aryl and R⁴ is —OR⁵, wherein R⁵ is independently ˜(CH₂)ₜ˜cycloalkyl or ˜(CH₂)ₜ˜heteroaryl wherein t is zero and said cycloalkyl or heteroaryl is optionally substituted with one to three R⁶. In another example of this embodiment, A is aryl and R⁴ is —(CH₂)ₜ-aryl wherein t is zero and the aryl is optionally substituted by one to three cyano, alkyl, halogen, or —OR⁵. In another example of this embodiment, A is aryl and R⁴ is ˜(CH₂)ₜ˜heteroaryl wherein t is zero and the heteroaryl is optionally substituted by one to three cyano, alkyl, halogen, or ˜OR⁵. In another example of this embodiment, A is heteroaryl and R⁴ is —OR⁵, wherein R⁵ is independently —(CH₂)ₜ-cycloalkyl or —(CH₂)ₜ-heteroaryl wherein t is zero and said cycloalkyl or heteroaryl is optionally substituted with one to three R⁶. In another example of this embodiment, A is heteroaryl and R⁴ is ˜(CH₂)ₜ˜aryl wherein t is zero and the aryl is optionally substituted by one to three cyano, alkyl, halogen, or —OR⁵, In another example of this embodiment, A is heteroaryl and R⁴ is —(CH₂)ₜ-heteroaryl wherein t is zero and the heteroaryl is optionally substituted by one to three cyano, alkyl, halogen, or ˜OR⁵.

In another embodiment of the invention A is aryl, heteroaryl, cycloalkyl or heterocycloalkyl, and A is optionally substituted with two R⁴ substituents. In one example of this embodiment, each R⁴ is independently alkyl, halogen, cyano, ˜SO₂NHR⁷, ˜CON(R⁷)₂, ˜N(R⁷)COR⁷, ˜SO₂N(R⁷)₂, ˜N(R⁷)SO₂R⁷, ˜COR⁷, ˜SO₂R⁷, —(CH₂)ₜ-cycloalkyl, —(CH₂)ₜ-heterocycloalkyl, —(CH₂)ₜ-aryl, —(CH₂)ₜ-heteroaryl, —(CH₂)ₜ—N(R⁷)₂, or —(CH₂)ₜ—OR⁵ wherein each R⁴ alkyl, —(CH₂)ₜ-cycloalkyl, —(CH₂)ₜ-heterocycloalkyl, ˜(CH₂)ₜ˜aryl, or ˜(CH₂)ₜ˜heteroaryl is optionally independently substituted by one to three cyano, alkyl, halogen, or —OR⁵. In another example of this embodiment, each R⁴ is alkyl optionally independently substituted by one to three cyano, alkyl, halogen, or —OR⁵. In another example of this embodiment, A is aryl or heteroaryl, and each R⁴ is independently alkyl, halogen, cyano, ˜SO₂NHR⁷, ˜CON(R⁷)₂, ˜N(R⁷)COR⁷, ˜SO₂N(R⁷)₂, ˜N(R⁷)SO₂R⁷, ˜COR⁷, ˜SO₂R⁷, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, ˜(CH₂)ₜ˜N(R⁷)₂, or —(CH₂)ₜ—OR⁵ wherein each R⁴ alkyl, —(CH₂)ₜ-cycloalkyl, —(CH₂)ₜ-heterocycloalkyl, ˜(CH₂)ₜ˜aryl, or ˜(CH₂)ₜ˜heteroaryl is optionally independently substituted by one to three cyano, alkyl, halogen, or ˜OR⁵. In an example of this embodiment, each R⁴ is alkyl optionally independently substituted by one to three cyano, alkyl, halogen, or —OR⁵. In another example of this embodiment, each R⁴ is independently alkyl, halogen, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl, wherein each R⁴ —(CH₂)ₜ-cycloalkyl, —(CH₂)ₜ-heterocycloalkyl, —(CH₂)ₜ-aryl, or —(CH₂)ₜ-heteroaryl is optionally independently substituted by one to three cyano, alkyl, halogen, or —OR⁵. In one example of this embodiment, A is aryl and at least one R⁴ is —(CH₂)ₜ-aryl wherein t is zero and the aryl is optionally substituted by one to three cyano, alkyl, halogen, or ˜OR⁵. In another example of this embodiment, A is aryl and each R⁴ is ˜OR⁵. In another example of this embodiment, A is heteroaryl and at least one R⁴ is —(CH₂)ₜ-aryl wherein t is zero and the heteroaryl is optionally substituted by one to three cyano, alkyl, halogen, or —OR⁵. In another example of this embodiment, A is heteroaryl and each R⁴ is ˜OR⁵.

In another embodiment of the invention, A is aryl, heteroaryl, cycloalkyl or heterocycloalkyl, and A is optionally substituted with three R⁴ substituents. In one example of this embodiment, each R⁴ is independently alkyl, halogen, cyano, ˜SO₂NHR⁷, ˜CON(R⁷)₂, ˜N(R⁷)COR⁷, ˜SO₂N(R⁷)₂, ˜N(R⁷)SO₂R⁷, ˜COR⁷, ˜SO₂R⁷, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, ˜(CH₂)ₜ˜N(R⁷)₂, or —(CH₂)ₜ—OR⁵ wherein each R⁴ alkyl, —(CH₂)ₜ-cycloalkyl, —(CH₂)ₜ-heterocycloalkyl, ˜(CH₂)ₜ˜aryl, or ˜(CH₂)ₜ˜heteroaryl is optionally independently substituted by cyano, alkyl, halogen, or ˜OR⁵. In one example of this embodiment, A is aryl or heteroaryl and each R⁴ is independently alkyl, halogen, cyano, —SO₂NHR⁷, —CON(R⁷)₂, —N(R⁷)COR⁷, —SO₂N(R⁷)₂, —N(R⁷)SO₂R⁷, —COR⁷, —SO₂R⁷, —(CH₂)ₜ-cycloalkyl, ˜(CH₂)ₜ˜heterocycloalkyl, ˜(CH₂)ₜ˜aryl, ˜(CH₂)ₜ˜heteroaryl, ˜(CH₂)ₜ˜N(R⁷)₂, or —(CH₂)ₜ—OR⁵ wherein each R⁴ alkyl, —(CH₂)ₜ-cycloalkyl, —(CH₂)ₜ-heterocycloalkyl, —(CH₂)ₜ-aryl, or —(CH₂)ₜ-heteroaryl is optionally independently substituted by one to three cyano, alkyl, halogen, or —OR⁵. In another example of this embodiment, each R⁴ is alkyl optionally independently substituted by one to three cyano, alkyl, halogen, or ˜OR⁵. In another example of this embodiment, each R⁴ is independently halogen, ˜OR⁵, cyano, trifluoroalkyl, ˜(CH₂)ₜ˜cycloalkyl, ˜(CH₂)ₜ˜heterocycloalkyl, ˜(CH₂)ₜ˜aryl, or —(CH₂)ₜ-heteroaryl, wherein each R⁴—(CH₂)ₜ-cycloalkyl, —(CH₂)ₜ-heterocycloalkyl, ˜(CH₂)ₜ˜aryl, or (CH₂)ₜ˜heteroaryl is optionally independently substituted by one to three cyano, alkyl, halogen, or ˜OR⁵. In another example of this embodiment, at least one R⁴ is —(CH₂)ₜ-heterocycloalkyl wherein t is zero and the heterocycloalkyl is pyrrolidinyl, piperidinyl, or morpholinyl, and is optionally independently substituted by cyano, alkyl, halogen, or ˜OR⁵.

In another embodiment of the invention, B is aryl. Examples of said embodiment include but are not limited to:
(5R,7S)-8-(4-Hydroxy-3-isopropoxy-benzyl)-7-methyl-1-phenyl-1,8-diaza-spiro[4.5]decan-2-one, and
(5R,7S)˜8˜(4˜Hydroxy˜3˜isopropoxy˜benzyl)˜7˜methyl˜1˜phenyl˜1,8˜diaza˜spiro[4.5]dec-3-en-2-one.

In another example of this invention, B is substituted with one to three R³ substituents. Examples of this embodiment include but are not limited to:
N˜{4˜[(5R,7S)˜8˜(4˜Hydroxy˜3˜isopropoxy˜benzyl)˜7˜methyl˜2˜oxo˜8˜diaza˜spiro[4.5]dec-1-yl]-phenyl}-acetamide;
(5R,7S)-1-Biphenyl-2-yl-8-(4-hydroxy-3-isopropoxy-benzyl)-7-methyl-1,8-diaza-spiro[4,5]decan-2-one;
(5R,7S)˜8˜(4˜Hydroxy˜3˜isopropoxy˜benzyl)˜7˜methyl˜1˜(3˜trifluoromethyl˜phenyl)˜1,8˜diaza˜spiro[4.5]decan˜2˜one;
3-[(5R,7S)-8-(4-Hydroxy-3-isopropoxy-benzyl)-7-methyl-2-oxo-1,8-diaza-spiro[4.5]dec˜1˜yl]˜benzonitrile;
(5R,7S)˜8˜(4˜Hydroxy˜3˜isopropoxy˜benzyl)˜1˜(4˜methoxy˜phenyl)˜7˜methyl˜1,8-diaza-spiro[4.5]decan-2-one; and
2′-[(5R,7S)-8-(4-Hydroxy-3-isopropoxy-benzyl)-7-methyl-2-oxo-1,8-diaza-spiro[4.5]dec˜1˜yl]˜biphenyl˜4˜sulfonic acid dimethylamide.

In one example of this invention, B is substituted with only one R³ substituent and R³ is halogen. Examples of said embodiment include but are not limited to:
(5R,7S)-1-(2-Fluoro-phenyl)-8-(4-hydroxy-3-isopropoxy-benzyl)-7-methyl-1,8-diaza˜spiro[4,5]decan˜2˜one;
(5R,7S)˜1˜(4˜Fluoro˜phenyl)˜8˜(4˜hydroxy˜3˜isopropoxy˜benzyl)˜7˜methyl˜1,8˜diaza˜spiro[4,5]decan˜2˜one;
(5R,7S)-1-(2-Chloro-phenyl)-8-(4-hydroxy-3-isopropoxy-benzyl)-7-methyl-1,8-diaza˜spiro[4.5]decan˜2˜one; and
(5R,7S)˜1˜(4˜Chloro˜phenyl)-8˜(4˜hydroxy˜3˜isopropoxy˜benzyl)˜7˜methyl˜1,8, diaza-spiro[4,5]decan-2-one,

In one example of this invention, B is cycloalkyl. An example of this embodiment includes but is not limited to (5R,7S)˜1˜Cyclohexyl˜8˜(4˜hydroxy˜3˜isopropoxy-benzyl)-7-methyl-1,8-diaza-spiro[4,5]decan-2-one.

In another example of this invention, B is alkyl. An example of this embodiment includes but is not limited to (5R,7S)-8-(4-Hydroxy-3-isopropoxy-benzyl)˜1˜isopropyl˜7˜methyl˜1,8˜diaza˜spiro[4,5]decan˜2˜one.

In another embodiment of this invention, B is heterocycloalkyl. An example of this embodiment includes but is not limited to (5R,7S)-8-(4-Hydroxy-3-isopropoxy-benzyl)-7-methyl-1-(tetrahydro-pyran-4-yl)-1,8-diaza-spiro[45]decan-2-one.

In another embodiment of the invention, R² is alkyl.

In a further embodiment of the invention, the compound, including the pharmaceutically acceptable salts thereof, have the structure, where the substituents are defined above:

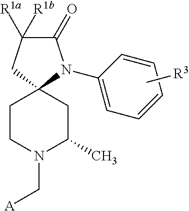

In another embodiment of the invention, the compound, including the pharmaceutically acceptable salts thereof, have the structure, where the substituents are defined above:

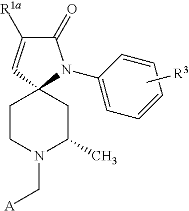

In another embodiment the present invention provides methods of treating neurological and psychiatric disorders comprising: administering to a patient in need thereof an amount of a compound of formula I effective in treating such disorders. Neurological and psychiatric disorders, include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi˜domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering to the mammal an effective amount of compound of formula 1 or pharmaceutically acceptable salt thereof. Accordingly, in one embodiment, the invention provides a method for treating a condition in a mammal, such as a human, selected from the conditions above, comprising administering a compound of formula I to the mammal. The mammal is preferably a mammal in need of such treatment. As examples, the invention provides a method for treating attention deficit/hyperactivity disorder, schizophrenia and Alzheimer's Disease.

In another embodiment the present invention provides methods of treating neurological and psychiatric disorders comprising: administering to a patient in need thereof an amount of a compound of formula I effective in treating such disorders. The compound of formula I is optionally used in combination with another active agent. Such an active agent may be, for example, an atypical antipsychotic, a cholinesterase inhibitor, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

The invention is also directed to a pharmaceutical composition comprising a compound of formula I, and a pharmaceutically acceptable carrier. The composition may be, for example, a composition for treating a condition selected from the group consisting of neurological and psychiatric disorders, including but not limited to: acute neurological and psychiatric disorders such as cerebral deficits subsequent to cardiac bypass surgery and grafting, stroke, cerebral ischemic, spinal cord trauma, head trauma, perinatal hypoxia, cardiac arrest, hypoglycemic neuronal damage, dementia, AIDS˜induced dementia, vascular dementia, mixed dementias, age˜associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug˜induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi˜domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering an effective amount of compound of formula 1 or pharmaceutically acceptable salt thereof, and a pharmaceutically acceptable carrier. The composition optionally further comprises an atypical antipsychotic, a cholinesterase inhibitor, dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

### Abbreviations and Definitions

TABLE A
Abbreviations

| | |
|---|---|
| Ac | Acetyl |
| APCI | Atmospheric pressure chemical ionization (in mass |
| | spectrometry) |
| Boc | tert-butoxycarbonyl |
| br | Broad |
| CD₃OD | Deuterated methanol |
| CDCl₃ | Deuterated chloroform |
| d | Doublet |
| dba | Dibenzylidene acetone |
| DCM | Dichloromethane |
| DMF | N,N-dirnethylformamide |
| dd | Doublet of doublets |
| DMSO | dimethyl sulphoxide |
| ES | Electrospray Ionization (in mass spectrometry) |
| Et₃N | Triethylamine |
| EtOAc | ethyl acetate |
| g | Gram (s) |
| h | Hour (s) |
| HPLC | High performance liquid chromatography |
| J | Coupling constant (in NMR) |
| LCMS | Liquid Chromatography-Mass Spectrometry |
| LDA | Lithium diisopropylamide |
| LRMS | Low Resolution Mass Spectrometry (electrospray or |
| | thermospray ionization positive scan) |
| LRMS | Low Resolution Mass Spectrometry |
| (ES) | (electrospray ionization |
| | negative scan) |
| m | Multipiet (spectral), meters (s), milli |
| m/z | mass to charge ratio (in mass spectrometry) |
| MeOH | Methanol |
| MHz | Megahertz |
| MS | Mass spectrometry |
| NMR | Nuclear Magnetic Resonance |
| ppm | Parts per million (in NMR) |
| psi | Pounds per square inch |
| q | Quartet |
| s | Singlet |
| t | Triplet |
| Tf | Trifluoromethanesulfonyl (triflyl) |
| TFA | trifluoroacetic acid |
| THF | Tetrahydrofuran |
| TLC | Thin layer chromatography |
| TMHD | 2,2,6,6-Tetramethyl-3,5-heptanedione |
| Vol. | Volume |
| δ | Chemical shift |

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert˜butyl), pentyl, iso˜amyl, hexyl and the like.

The term “benzyl” refers to methyl radical substituted with phenyl, i.e., the following structure:

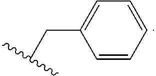

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkylene moiety” refers to a carbocyclic substituent obtained by removing two hydrogen atoms from a saturated carbocyclic molecule and having three to fourteen carbon atoms. In one embodiment, a cycloalkylene substituent has three to ten carbon atoms. Examples of cycloalkylene include the following:

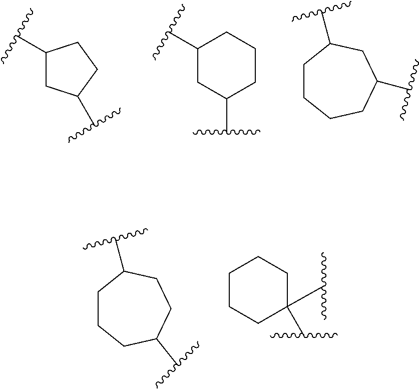

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5˜10˜membered heteroaromatic ring may be optionally substituted with halogen, C₁˜C₆ alkyl, C₃˜C₁₀ cycloalkyl, or ═O.

A cycloalkyl may be a single ring, which typically contains from 3 to 6 ring atoms. Examples include cyclopropyl, cyclobutyl, cyclopentyl, and cyclohexyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄-C₁₀ carbocyclic or 4- to 10-membered heterocyclic ring may be optionally substituted with halogen, C₁˜C₆ alkyl, C₃˜C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (i.e., alkyl, alkenyl, cycloalkyl, cycloalkenyl, aryl, etc.) is indicated by the prefix “Cₓ-C_{y}-,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃˜C₆˜cycloalkyl refers to saturated cycloalkyl containing from 3 to 6 carbon ring atoms.

In some instances, the number of atoms in a cyclic substituent containing one or more heteroatoms (i.e., heteroaryl or heterocycloalkyl) is indicated by the prefix “X-Y˜membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as ˜H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “cyano” (also referred to as “nitrile”) means —CN, which also may be depicted:

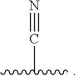

The term “carbonyl” means —C(O)—, which also may be depicted as:

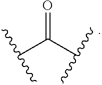

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

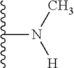

and dialkylamino such as dimethylamino, (exemplified by the formula ˜N(CH₃)₂), which may also be depicted:

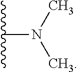

The term “halogen” refers to fluorine (which may be depicted as ˜F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl, Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The term “oxo” refers to ═O.

The term “oxy” refers to an ether substituent, and may be depicted as —O—.

The term “alkoxy” refers to an alkyl linked to an oxygen, which may also be represented as:

—O—R, wherein the R represents the alkyl group. Examples of alkoxy include methoxy, ethoxy, propoxy and butoxy.

The term “heterocycloalkyl” refers to a substituent obtained by removing a hydrogen from a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (i.e., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆˜C₁₀ aromatic ring or to a 5- to 10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one more substituents, the one or more substituents, unless otherwise specified, are each bound to a heteroatom of the heterocycloalkyl group or to a carbon atom of the heterocycloalkyl group. The fused C₆-C₁₀ aromatic ring or 5- to 10-membered heteroaromatic ring may be optionally substituted with halogen, C₁˜C₆ alkyl, C₃˜C₁₀ cycloalkyl, C₁˜C₆ alkoxy, or ═O.

The term “heterocycloalkylene moiety” refers to a substituent obtained by removing two hydrogen atoms from a saturated or partially saturated ring structure containing a total of 3 to 14 ring atoms, where at least one of the ring atoms is a heteroatom. In one embodiment, a heterocycloalkylene substituent has three to ten ring atoms. Examples of heterocycloalkylene include the following:

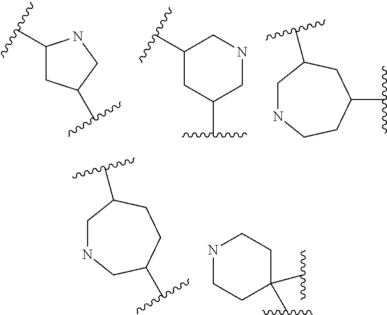

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6˜membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N˜oxide ring.

Examples of single˜ring heteroaryls and heterocycloalkyls include furanyl, dihydrofuranyl, tetrahydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2˜diazinyl”), pyrimidinyl (also known as “1,3˜diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3˜oxazinyl, 1,3,2˜oxazinyl, 1,3,6˜oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, purinyl, naphthyridinyl, pyridopyridinyl (including pyrido[3,4˜b]˜pyridinyl, pyrido[3,2˜b]˜pyridinyl, or pyrido[4,3˜b]˜pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6˜dihydro˜4H˜imidazo[4,5,1˜ij]quinoline, 4,5˜dihydroimidazo[4,5,1˜hi]indole, 4,5,6,7˜tetrahydroimidazo[4,5,1˜jk][1]benzazepine, and dibenzofuranyl.

Other examples of fused˜ring heteroaryls include benzo˜fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1˜benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3˜benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2˜benzisoxazinyl or 1,4˜benzisoxazinyl), tetrahydroisoquinolinyl carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4˜ to 10˜membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocyclic or 4- to 10-membered heterocyclic ring may be optionally substituted with halogen, C₁-C₆ alkyl, C₃˜C₁₀ cycloalkyl, or ═O.

Additional examples of heteroaryls and heterocycloalkyls include: 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]˜dioxalanyl, [1,3]˜dithiolanyl, [1,3]˜dioxanyl, 2˜tetrahydrothiophenyl, 3˜tetrahydrothiophenyl, 2˜morpholinyl, 3˜morpholinyl, 4˜morpholinyl, 2˜thiomorpholinyl, 3˜thiomorpholinyl, 4˜thiomorpholinyl, 1˜pyrrolidinyl, 2˜pyrrolidinyl, 3˜pyrrolidinyl, 1˜piperazinyl, 2-piperazinyl, 1-piperidinyl, 2-piperidinyl, 3-piperidinyl, 4-piperidinyl, 4-thiazolidinyl, diazolonyl, N˜substituted diazolonyl, 1˜phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-alpha]pyridine, benzothianyl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C˜attached or N˜attached where such is possible. For instance, a group derived from pyrrole may be pyrrol˜1˜yl (N˜attached) or pyrrol-3˜yl (C˜attached). Further, a group derived from imidazole may be imidazol-1-yl (N-attached) or imidazol-2˜yl (C˜attached).

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non˜hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non˜hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non˜hydrogen substituents, then any heteroaryl with less than 3 substitutable positions would be optionally substituted by up to only as many non˜hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non˜hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi˜moiety substituent only applies to the first moiety. To illustrate, the term “alkylcycloalkyl” contains two moieties: alkyl and cycloalkyl. Thus, a C₁-C₆- prefix on C₁-C₆-alkylcycloalkyl means that the alkyl moiety of the alkylcycloalkyl contains from 1 to 6 carbon atoms; the C₁-C₆- prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution only occurs on the alkyl moiety, the substituent would be described as “alkoxyhaloalkyl.” If the halogen substitution occurs on both the alkyl moiety and the alkoxy moiety, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A˜B˜C, moiety C is attached to the remainder of the molecule. In a substituent A˜B˜C˜D, moiety D is attached to the remainder of the molecule. Similarly, in a substituent aminocarbonylmethyl, the methyl moiety is attached to the remainder of the molecule, where the substituent may also be depicted as

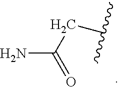

In a substituent trifluoromethylaminocarbonyl, the carbonyl moiety is attached to the remainder of the molecule, where the substituent may also be depicted as

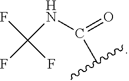

If substituents are described as being “independently selected” from a group, each substituent is selected independent of the other. Each substituent therefore may be identical to or different from the other substituent(s).

### Isomers

When an asymmetric center is present in a compound of formula I, hereinafter referred to as the compound of the invention, the compound may exist in the form of optical isomers (enantiomers). In one embodiment, the present invention comprises enantiomers and mixtures, including racemic mixtures of the compounds of formula I. In another embodiment, for compounds of formulae that contain more than one asymmetric center, the present invention comprises diastereomeric forms (individual diastereomers and mixtures thereof) of compounds. When a compound of formula I contains an alkenyl group or moiety, geometric isomers may arise.

### Tautomeric Forms

The present invention comprises the tautomeric forms of compounds of formula I. Where structural isomers are interconvertible via a low energy barrier, tautomeric isomerism (‘tautomerism’) can occur. This can take the form of proton tautomerism in compounds of formula I containing, for example, an imino, keto, or oxime group, or so˜called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non˜toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p˜hydroxybenzoate, phenylacetate, mandelate, embonate (pamoate), methanesulfonate, ethanesulfonate, benzenesulfonate, pantothenate, toluenesulfonate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2˜naphthalesulfonate, oxalate, palmoate, pectinate, 3˜phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non˜toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N˜dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hem sulphate and hemicalcium salts.

### Prodrugs

Also within the scope of the present invention are so-called “prodrugs” of the compound of the invention. Thus, certain derivatives of the compound of the invention which may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs in accordance with the invention can, for example, be produced by replacing appropriate functionalities present in the compounds of any of formula I with certain moieties known to those skilled in the art as “pro-moieties” as described, for example, in “Design of Prodrugs” by H Bundgaard (Elsevier, 1985).

### Isotopes

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half˜life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### Administration and Dosing

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle˜free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment of the conditions recited herein.

### Pharmaceutical Compositions

For the treatment of the conditions referred to above, the compound of the invention can be administered as compound per se. Alternatively, pharmaceutically acceptable salts are suitable for medical applications because heir greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit˜dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled˜release formulation. In the case of capsules, tablets, and pills, the dosage forms also may comprise buffering agents or may be prepared with enteric coatings.

In another embodiment, oral administration may be in a liquid dose form, Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH˜adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2˜tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well˜known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention.

### General Synthetic Schemes

The compounds of the formula I may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

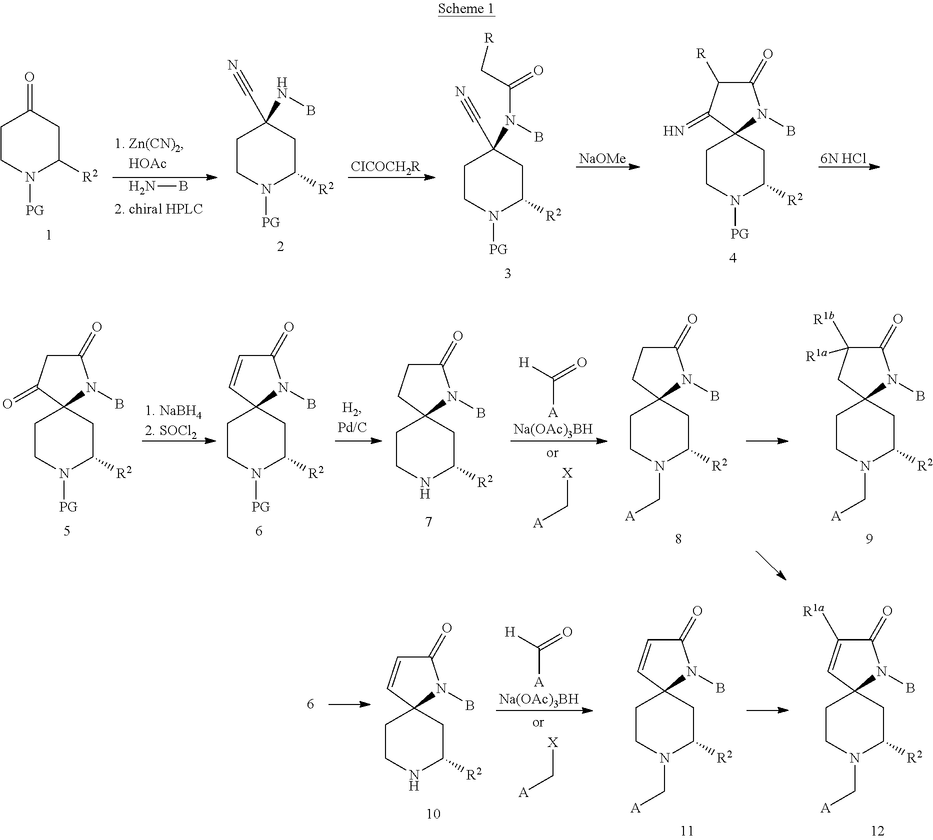

Scheme 1 illustrates the synthesis of lactam derivatives depicted by Formula I employing methods well known to one skilled in the art. Referring to scheme 1, Strecker reaction of an appropriately protected chiral piperidinone with zinc cyanide in acetic acid followed by chiral separation provides chiral compounds 2. Acylation of amine 2 with an appropriate acyl chloride provides compounds 3. Formation of the keto-amide 5 is accomplished by base catalyzed closure of 4 followed by decarboxylation/hydrolysis. Reduction of the carbonyl group of 5 with sodium borohydride followed by conversion to the chloride and elimation provides 6. Reduction of the enone and removal of the protecting group (in the case of Cbz) is accomplished with hydrogenation to provide lactam 7, Reductive amination of 7 with an aldehyde and sodium triacetoxyborohydride or alkylation of 7 with a halide (X═Cl, Br, I) and base such as sodium hydride provides compound 8. Installation of R^{1a}/R^{1b} to provide compound 9 is accomplished using methods known to one skilled in the art. Alternatively, removal of the protecting group of compound 6 (in the case of Cbz this is accomplished with 6N HCl) provides enone 10. Reductive amination of 10 with an aldehyde and sodium triacetoxyborohydride or alkylation of 10 with a halide (X═Cl, Br, I) and base such as sodium hydride provides compound 11. Synthesis of compound 12 is accomplished using methods known to one skilled in the art from compounds 8 or 11.

### EXPERIMENTAL PROCEDURES AND WORKING EXAMPLES

The following illustrate the synthesis of various compounds of the present invention. Additional compounds within the scope of this invention may be prepared using the methods illustrated in these Examples, either alone or in combination with techniques generally known in the art.

Experiments were generally carried out under inert atmosphere (nitrogen or argon), particularly in cases where oxygen˜ or moisture˜sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS) or atmospheric pressure chemical ionization (APCI) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluants/gradients were chosen to provide appropriate Rᵢs or retention times.

### Preparation 1

### Racemic (5R,7S),(5S,7R)˜1˜(3˜fluorophenyl)˜7˜methyl˜1,8˜diazaspiro[4.5]dec˜3-en-2-one (P1)

Step 1. Synthesis of benzyl 2˜methyl˜4˜oxo˜3,4˜dihydropyridine˜1(2H)˜carboxylate (C1). Benzyl chloroformate (235 g, 1.38 mol) was added drop-wise to a chilled solution of 4-methoxypyridine (150 g, 1.38 mol) and triethylamine (19 mL, 0.137 mol) in anhydrous tetrahydrofuran (6 L), while keeping the temperature below ˜50° C. A white precipitate formed. After completion of the addition, the resulting suspension was stirred at −60° C. for 20 minutes. Methylmagnesium bromide (3.0 M in diethyl ether, 650 mL, 1.95 mol) was then added drop˜wise at ˜60° C.˜˜50° C. The reaction mixture was stirred at room temperature overnight, at which time thin layer chromatography (petroleum ether/ethyl acetate=1:1) indicated that the reaction was complete. After quenching the reaction with 1 N aqueous hydrochloric acid (500 mL), the color of the reaction mixture became brown˜black. The organic layer was separated and concentrated in vacuo, and the residue and the aqueous layer were extracted with ethyl acetate (2×2 L). The combined organic layers were washed with saturated aqueous sodium chloride solution (500 dried over sodium sulfate and evaporated to dryness to give crude C1, which was used in the next step without purification, Yield: 1500 g, 4 batches.

Step 2. Synthesis of benzyl 2˜methyl˜4˜oxopiperidine˜1˜carboxylate (C2). To a stirred solution of compound C1 (750 g, 3.06 mol) in acetic acid (2.8 L) at 100° C. was added zinc powder (795 g, 12.2 mol) over 4 hours in four portions. The reaction mixture became yellow. After completion of the addition, the reaction mixture was stirred at 110° C. for 1 hour. The mixture was filtered through Celite, the filtrate was concentrated in vacuo, and the residue was diluted with water (2 L), and extracted with ethyl acetate (3 L). The combined organic layers were basified with solid potassium carbonate to pH 7˜8, then washed with saturated aqueous sodium chloride solution (1 L), dried over sodium sulfate and evaporated to dryness to give crude C2 as a brown-black oil, which was purified by column chromatography on silica gel (Gradient: 0-10% ethyl acetate in petroleum ether) to afford C2 as a light yellow oil. Yield: 836 g from 2 batches, 3.38 mol, 61% over two steps. ¹H NMR (400 MHz, CDCl₃): δ 7.40˜7.32 (m, 5H), 5.18 (s, 2H), 4.79 (m, 1H), 4.34˜4.30 (m, 1H), 3.42-3.35 (m, 1H), 2.71-2.66 (dd, 1H), 2.54-2.45 (m, 1H), 2.38-2.25 (m, 2H), 1.21-1.20 (d, 3H).

Step 3. Synthesis of racemic benzyl (2S,4R)(2R,4S)˜4˜cyano˜4˜[(3˜fluorophenyl)amino]˜2˜methylpiperidine˜1˜carboxylate (C3). 3˜Fluoroaniline (376 g, 3.38 mol) was added drop˜wise to a solution of compound C2 (418 g, 1.69 mol) in acetic acid (3 L) at room temperature. Zinc cyanide (430 g, 3.66 mol) was then added in portions. The reaction mixture was stirred at worn temperature for 18 hours, at which point thin layer chromatography (petroleum ether/ethyl acetate=4:1) showed the reaction was complete. The mixture was cooled to 0° C., and aqueous ammonium hydroxide solution (2 L) was added drop˜wise until pH=7˜8. The resulting mixture was extracted with ethyl acetate (3×2 L). The combined organic layers were washed with saturated aqueous sodium chloride solution (1 L), dried over sodium sulfate and concentrated in vacuo to give crude C3 (530 g), which was purified by column chromatography on silica gel (Gradient 1:20 to 1:2 ethyl acetate/petroleum ether) to give C3 as a brown oil comprised of a mixture of diastereomers. Yield: 846 g, 2 batches, 2.30 mol, 68%. ¹H NMR (400 MHz, CDCl₃): δ 7.39˜7.31 (m, 5H), 7.24˜7.15 (m, 1H), 6.66˜6.59 (m, 3H), 5.15 (s, 2H), 4.63˜4.43 (2 multiplets, 1H), 4.28-4.02 (2 multiplets, 1H), 3.85-3.76 (2 broad singlets, 1H), 3.39-3.24 (m, 1H), 2.40˜2.18 (several multiplets, 3H), 1.83˜1.58 (2 multiplets, 1H), 1.41˜1.20 (2 doublets. 3H).

Step 4. Synthesis of racemic benzyl (2S,4R)(2R,4S)˜4˜cyano˜4˜[(3˜ethoxy˜3˜oxopropanoyl)(3-fluorophenyl)amino]-2-methylpiperidine-1-carboxylate (C4). 2,6-Dimethylpyridine (242 g, 2.26 mol) and ethyl 3˜chloro˜3˜oxopropanoate (255 g, 1.69 mol) were added to a solution of C3 (415.5 g, 1.13 mol) in anhydrous dichloromethane (2 L) at 10° C. The brown mixture was stirred at room temperature overnight Water (500 mL) was added at 15° C., and the organic layer was separated and washed with saturated aqueous sodium chloride solution (1 L), dried over sodium sulfate, filtered and evaporated to dryness to give crude product, which was purified by chromatography on silica gel (Eluant: 1:15 then 1:5 then 1:1 ethyl acetate:petroleum ether) to give compound C4 as a brown oil. Yield: 465 g, 2 batches, 0.965 mol, 43%. NMR data indicated that this material was a single diastereomer. ¹H NMR (400 MHz, CDCl₃): δ 7.48˜7.43 (m, 1H), 7.42˜7.30 (m, 5H), 7.23˜7.19 (m, 1H), 7.06˜6.92 (m, 2H), 5.14˜5.07 (m, 2H), 4.55 (br s, 1H), 4.24˜4.09 (m, 3H), 3.38˜3.31 (m, 1H), 3.14˜3.05 (m, 2H), 2.80˜2.76 (m, 1H), 2.17˜2.04 (m, 1H), 1.78˜1.72 (m, 1H), 1.48 (d, 3H), 1.46˜1.35 (m, 1H), 1.28˜1.20 (t, 3H).

Step 5. Synthesis of racemic 8˜benzyl 3˜ethyl (5R,7S)(5S,7R)˜4˜amino˜1˜(3˜fluorophenyl)˜7˜methyl˜2˜oxo˜1,8˜diazaspiro[4.5]dec˜3˜ene˜3,8˜dicarboxylate (C5). To a solution of compound C4 (450 g, 0.934 mol) in methanol (3.4 L) at 15° C. was added a solution of sodium methoxide (60.5 g, 1.12 mol) in methanol (600 mL). A yellow precipitate formed. After completion of the addition, the reaction mixture was stirred at room temperature for 40 minutes. Thin layer chromatography (petroleum ether/ethyl acetate=2:1) showed the starting material was completely consumed. The reaction was concentrated in vacuo to give crude product, which was suspended in methanol (100 mL) and water (2 L); this mixture was cooled to 5° C. and acidified with 1 N aqueous hydrochloric acid to pH 6. The solid was filtered and dried to obtain compound C5 as a white solid. Yield: 445 g, 0.923 mol, 99%. ¹H NMR (400 MHz, CDCl₃): δ 7.35-7.33 (m, 3H), 7.24-7.22 (m, 3H), 6.95-6.93 (m, 3H), 4.93-4.90 (d, 1H), 4.72-4.68 (d, 1H), 4.26-4.20 (m, 1H), 3.87-3.83 (m, 1H), 3.36-3.29 (m, 1H), 2.98-2.90 (m, 1H), 2.41-2.33 (m, 1H), 2.10-2.01 (m, 1H), 1.92-1.86 (m, 1H), 1.79-1.72 (m, 1H), 1.30˜1.26 (t, 3H), 0.95˜0.92 (m, 3H).

Step 6. Synthesis of racemic (5R,7S)(5S,7R)˜1˜(3˜fluorophenyl)˜7˜methyl˜1,8˜diazaspiro[4,5]decane˜2,4˜dione hydrochloride (C6). Compound C5 (217 g, 0.45 mol) was added portion-wise to 6 N aqueous hydrochloric acid (2 L) at room temperature, and the mixture was heated to reflux for 5 hours. The mixture was cooled to room temperature, and concentrated in vacuo to give crude C6 as a brown solid, which was used in the next step without purification. Yield: 282 g, 2 batches.

Step 7. Synthesis of racemic benzyl (5R,7S)(5S,7R)-1-(3-fluorophenyl)-7-methyl-2,4-dioxo-1,8-diazaspiro[4,5]decane-8-carboxylate (C7). To a solution of C6 (140.5 g, 0.45 mol) in water/tetrahydrofuran (400 mL/800 mL) at 5° C. was added a solution of sodium hydroxide (90 g, 2.25 mol) in water (400 mL). The reaction became red˜brown. The reaction mixture was cooled to 0° C. and benzyl chloroformate (115.2 g, 0.67 mol) was added drop-wise. The solution, which became yellow in color, was stirred for 1 hour at 0° C., at which time thin layer chromatography (petroleum ether/ethyl acetate=1:2) showed the reaction was complete. Ethyl acetate (500 mL) was added and the aqueous layer was separated, cooled to 0° C., and acidified with 4 N aqueous hydrochloric acid to pH 2-3. The aqueous layer was then extracted with ethyl acetate (3×1 L) and the combined organic layers were washed with saturated aqueous sodium chloride solution (500 mL), dried over sodium sulfate and concentrated in vacuo to give C7 as a yellow solid. Yield: 84 g, 2 batches, 0.205 mol, 23% over too steps. ¹H NMR (400 MHz, CDCl₃): δ 7.46-7.40 (m, 1H), 7.37˜7.27 (m, 5H), 7.18˜7.13 (m, 1H), 6.93˜6.85 (m, 2H), 5.07˜4.99 (m, 2H), 4.36˜4.34 (m, 1H), 4.07˜4.03 (m, 1H), 3.55˜3.48 (m, 1H), 3.40˜3.18 (AB quartet, 2H), 2.05-1.96 (m, 2H), 1.86-1.82 (br d, 1H), 1.74-1.72 (m, 1H), 1.26-1.24 (d, 3H).

Step 8. Synthesis of racemic benzyl (5R,7S)(5S,7R)-1-(3-fluorophenyl)-4-hydroxy-7-methyl-2-oxo-1,8-diazaspiro[4.5]decane-8-carboxylate (C8). To a suspension of compound C7 (84 g, 0.205 mol) in methanol/tetrahydrofuran (2500 mL/500 mL) at 15° C. was added sodium borohydride (23.3 g, 0.614 mol) in portions. After completion of the addition, the solution was light yellow. The mixture was stirred at 15° C. for 1 hour, at which time thin layer chromatography (petroleum ether/ethyl acetate=1:1) showed the reaction was complete. The solvent was removed in vacuo and the residue was diluted with ethyl acetate (2 L). The mixture was washed with water (500 mL), then with saturated aqueous sodium chloride solution (500 mL), dried over sodium sulfate and concentrated under reduced pressure to give C8 as a yellow solid. Yield: 84 g, 0.204 mol, 99%.

Step 9. Synthesis of racemic benzyl (5R,7S)(5S,7R)˜1˜(3˜fluorophenyl)˜7˜methyl-2-oxo-1,8-diazaspiro[4.5]dec-3-ene-8-carboxylate (C9). Thionyl chloride (73.68 g, 0.614 mol) was added drop-wise to a solution of compound C8 (84 g, 0.204 mol) in pyridine (1.5 L) at 0° C. The mixture was stirred at room temperature for 1 hour, then heated to 50° C. for 5 hours. After cooling to room temperature, the solvent was removed in vacuo, and the residue was diluted with ethyl acetate (1 L) and washed with saturated aqueous sodium bicarbonate solution to pH 7. The organic layer was dried over sodium sulfate and evaporated to dryness to give crude product, which was purified by silica gel column chromatography to give C9 as a brown syrup. Yield: 62 g, 0.157 mol, 77%. LCMS m/z 395.1 (1). ¹H NMR (400 MHz, CDCl₃): δ 7.64-7.62 (d, 1H), 7.52-7.37 (m, 1H), 7.34-7.32 (m, 5H), 7.16-7.11 (m, 1H), 6.91-6.80 (m, 2H), 6.34˜6.32 (d, 1H), 5.08 (br s, 2H), 4.70˜4.64 (m, 1H), 4.24˜4.20 (m, 1H), 3.18˜3.09 (m, 1H), 2.13˜2.06 (m, 1H), 1.91˜1.87 (m, 1H), 1.69˜1.62 (m, 1H), 1.47˜1.44 (br d, 1H), 1.32˜1.30 (d, 3H).

Step 10. Synthesis of racemic (5R,7S)(5S,7R)˜1˜(3˜fluorophenyl)˜7˜methyl˜1,8˜diazaspiro[4.5]dec˜3˜en˜2˜one (P1). To a solution of C9 (20 g, 51 mmol) in methanol (20 mL) was added 6 N aqueous hydrochloric acid (200 mL) at room temperature. The reaction was then heated to reflux for 2 hours, during, which time the brown solution became yellow. Thin layer chromatography (petroleum ether/ethyl acetate=1:2) showed the reaction was complete. The mixture was concentrated to half of the initial volume, and then extracted with ethyl acetate (2×100 mL). These organic extracts were discarded. The aqueous layer was cooled to 10° C. and basified with saturated aqueous sodium hydroxide to pH 11, then extracted with ethyl acetate (5×200 mL). The organic layers were dried over sodium sulfate, filtered and evaporated to afford P1 as a red syrupy solid. Yield: 12 g, 46 mmol, 90%. LCMS m/z 261.3 (M+1). ¹H NMR (400 MHz, CDCl₃): δ 7.44-7.35 (m, 1H), 7.14-7.10 (m, 1H), 7.03-7.02 (d, 1H), 7.00-6.98 (d, 1H), 6.94-6.91 (m, 1H), 6.18-6.16 (d, 1H), 2.92-2.87 (m, 1H), 2.77-2.70 (m, 1H), 2.67-2.60 (m, 1H) 2.02-1.91 (m, 1H), 1.90-1.84 (m, 2H), 1.70˜1.61 (dd, 1H), 1.08˜1.02 (d, 3H).

### Preparation 2

### Racemic (5R,7S)(5S,7R)˜1˜(3˜fluorophenyl˜7˜methyl˜1,8˜diazaspiro[4.5]decan-2-one (P2)

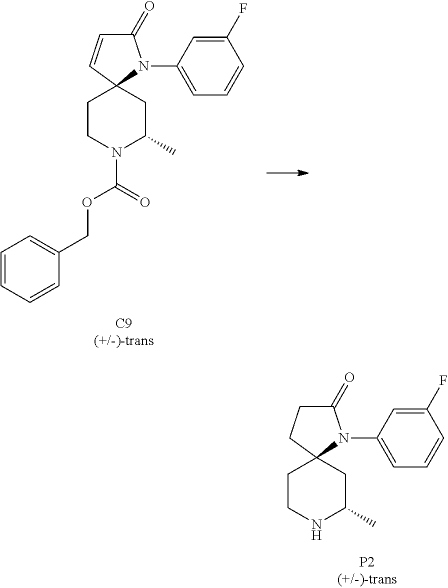

Synthesis of P2. A mixture of C9 (20 g, 51 mmol) and palladium hydroxide on carbon (2 g) in methanol (200 mL) was stirred under 45 psi of hydrogen at room temperature for 18 hours. Thin layer chromatography (petroleum ether/ethyl acetate=2:1 and dichloromethane/methanol=10:1) showed the reaction was complete. The reaction mixture was filtered and the filtrate was concentrated in vacuo: the residue was then diluted with ethyl acetate (100 mL) and water (100 mL). The mixture was cooled to 10° C. and acidified with 1 N aqueous hydrochloric acid to pH 2-3, after which the aqueous layer was separated and maintained at 10° C. It was then basified to pH 11 with saturated aqueous sodium hydroxide, and extracted with ethyl acetate (5×200 mL). These five organic layers were combined, dried over sodium sulfate and evaporated to give P2 as a red syrupy solid. Yield: 9.0 g, 34 mmol, 67%. LCMS m/z 2632 (M+1). ¹H NMR (400 MHz, CDCl₃): δ 7.40-7.34 (m, 1H), 7.10-7.05 (m, 1H), 6.95˜6.93 (m, 1H), 6.90˜6.86 (m, 1H), 2.87˜2.81 (m, 1H), 2.76˜2.69 (m, 1H), 2.61-2.54 (m, 3H), 2.16-1.97 (m, 4H), 1.79-1.70 (m, 1H), 1.50-1.41 (rid, 1H), 1.08-1.02 (d, 3H),

### Preparation 3

### (5R,7S)˜1˜(3˜Fluorophenyl)˜7˜methyl˜1,8˜diazaspiro[4.5]dec˜3˜en˜2˜one (P3)

Step 1. Synthesis of benzyl (2S,4R)-4-cyano-4-[(3-fluorophenyl)amino]-2-methylpiperidine˜1˜carboxylate (C10). A solution of benzyl (2S)˜2˜methyl˜4˜oxopiperidine˜1˜carboxylate (see C. Coburn et al. PCT Patent Application Publication WO 2007011810 A1 20070125) (31 g, 125 mmol) in acetic acid (250 mL) was treated with 3-fluoroaniline (24.1 mL, 250 mmol) followed by zinc cyanide (36.8 g, 313 mmol). The reaction mixture was allowed to stir at room temperature for 18 hours, at which time it was cooled in an ice bath and slowly basified with aqueous ammonium hydroxide solution. The resulting mixture was extracted three times with dichloromethane, and the combined organic layers were dried and concentrated in vacuo. Purification of the residue by silica gel chromatography (Eluant: 20% to 40% ethyl acetate in heptane) afforded a mixture of C10 and its isomer benzyl (2S,4S)-4-cyano˜4˜[(3˜fluorophenyl)amino]˜2˜methylpiperidine˜1˜carboxylate (C11) as an oil. Yield: 36 g, 98 mmol, 78%. This material was subjected to chromatography using a Chiralcel OJ-H column, 5 μm, 30×250 mm (Mobile phase: 70/30 CO₂/methanol; Flow rate: 120 g/min) to afford 14.6 g (32%) of C10 as a oil. Retention time: 3.45-4.46 min, MS (APCI) m/z 341.1 (M−CN)⁺. ¹H NMR (400 MHz, CDCl₃) δ 1.49 (d, J=7.3 Hz, 3H), 1.70 (ddd, J=13.3, 13.3, 4.4 Hz, 1H), 1.89 (dd, J=13.9, 6.6 Hz, 1H), 2.46 (m, 2H), 3.35 (m, 1H), 3.73 (br s, 1H), 4.28 (m, 1H), 4.63 (m, 1H), 5.16 (AB quartet, J=12 Hz, 2H), 6.60-6.67 (m, 3H), 7.21 (m, 1H), 7.37 (m, 5H).

Step 2. Synthesis of benzyl (2S,4R)-4-cyano-4-[(3-ethoxy-3-oxopropanoyl)(3-fluorophenyl)amino]-2-methylpiperidine-1-carboxylate (C12). 2,6-Dimethylpyridine (99%, 4.80 mL, 40.8 mmol) was added to a solution of C10 (10 g, 27 mmol) in dichloromethane (136 mL). Ethyl 3˜chloro˜3˜oxopropanoate (4.48 mL, 35.4 mmol) was then added drop˜wise from an addition funnel, and the reaction mixture was allowed to stir at room temperature for 4 hours. The mixture was diluted with dichloromethane (30 mL), washed with water (80 mL), with saturated aqueous sodium chloride solution (80 mL), and then dried over sodium sulfate. Filtration and removal of solvent in vacuo was followed by chromatographic purification on silica gel (Eluant: 30% ethyl acetate in heptane) to provide C12 (6.64 g) as a yellow oil. Mixed fractions were rechromatographed to provide additional C12. Total yield: 8.24 g, 17.1 mmol, 63%. LCMS m/z 482.0 (M+1). ¹H NMR (500 MHz, CD₃OD) δ 1.20 (t, J=7.1 Hz, 3H), 1.44 (2 doublets, J=7.3, 7.3 Hz, 3H), 1.46 (m, 1H), 1.90 (m, 1H), 2.16 (m, 1H), 2.76 (m, 1H), 3.14 (s, 2H), 3.33 (m, assumed 1H, partially obscured by solvent peak), 4.09 (2 quartets, J=7.1, 7.1 Hz, 2H), 4.15 (m, 1H), 4.54 (m, 1H), 5.10 (m, 2H), 7.16 (m, 2H), 7.29-7.35 (m, 6H), 7.53 (m, 1H),

Step 3. Synthesis of 8˜benzyl 3˜ethyl (5R,7S)˜4˜amino˜1˜(3˜fluorophenyl)˜7-methyl˜2˜oxo˜1,8˜diazaspiro[4.5]dec˜3˜ene˜3,8˜dicarboxylate (C13). Sodium metal (426 mg, 18.5 mmol, prewashed with heptane) was added to methanol (12 mL) and allowed to react completely. This solution of sodium methoxide was then added to a 0° C. solution of C12 (6.64 g, 14.2 mmol) in methanol (45 mL). The reaction mixture was allowed to warm to room temperature, stirred for 45 minutes, and concentrated to provide C13 as a yellow paste, which was taken on to the next transformation without purification. Yield: 6.84 g, 14.2 mmol, 100%.

LCMS m/z 482.1 (M+1). ¹H NMR (500 MHz, CD₃OD) δ 1.02 (d, J=6.1 Hz, 3H), 1.31 (t, J=7.1 Hz, 3H), 2.01 (dd, J=14.7, 11.3 Hz, 1H), 2.12 (dd, J=14.6, 6.8 Hz, 1H), 2.19 (dd, J=15.4, 4.8 Hz, 1H), 2.56 (m, 1H), 3.18 (m, 1H), 3.44 (m, 1H), 3.97 (dd, J=14.0, 6.6 Hz, 1H), 4.27 (q, J=7.1 Hz, 2H), 4.71 (m, 1H), 4.95 (br d, J=12.0 Hz, 1H), 7.05 (m, 1H), 7.10 (br d, J=8.3 Hz, 2H), 7.26 (m, 2H), 7.28˜7.37 (m, 4H).

Step 4. Synthesis of (5R,7S)-1-(3-fluorophenyl)-7-methyl-1,8-diazaspiro[4.5]decane-2,4-dione hydrochloride (C14). Compound C13 (8.0 g, 17 mmol) was added in portions to an aqueous 6 N solution of hydrochloric acid (130 mL), and the yellow suspension was heated at reflux for 28 hours. After cooling to room temperature, the mixture was azeotroped five times with toluene, then dried under high vacuum for 18 hours to provide C14 as a gray˜green solid. Yield: 6.3 g, assumed quantitative. LCMS m/z 277.1 (M+1).

Step 5. Synthesis of benzyl (5R,7S)˜1˜(3˜fluorophenyl)˜7˜methyl˜2,4˜dioxo˜1,8˜diazaspiro[4.5]decane˜8˜carboxylate (C15). A solution of C14 from the previous step (4.73 g, <15.1 mmol) in tetrahydrofuran (40 mL) and water (20 mL) was cooled to 0° C. and treated with a solution of sodium hydroxide (4.11 g, 103 mmol) in water (19 mL). Benzyl chloroformate (95%, 4.61 mL, 30.8 mmol) was added, and the resulting solution was stirred at 0° C. for 2 hours. Another portion of benzyl chloroformate (95%, 1.28 mL, 8.6 mmol) was added, and the reaction was stirred for an additional 2 hours at 0° C. After concentration in vacuo to remove tetrahydrofuran, the residue was diluted with water (50 and extracted three times with dichloromethane. The combined organic layers were dried over sodium sulfate, filtered and concentrated in vacuo, and the crude product was purified twice by chromatography on silica gel (Gradient: 5% to 100% ethyl acetate in heptane, then 30% to 100% ethyl acetate in heptane). The resulting material (5.78 g) was identified as the enol benzyl carbonate by mass spectroscopic and NMR analysis. The bulk of this material (5.05 g) was dissolved in tetrahydrofuran (about 60 mL) and stirred with aqueous sodium hydroxide solution (1 N, 200 mL, 200 mmol) for 5 hours. The reaction mixture was then acidified to pH 2 with aqueous 1 N hydrochloric acid, and extracted twice with dichloromethane. The combined organic layers were dried over sodium sulfate, and concentrated to afford C15 as a brown oil, contaminated with extraneous aromatic material. Yield 4 g, <9.7 mmol. LCMS m/z 411.1 (M+1). ¹H NMR (400 MHz, CDCl₃) Product peaks only: δ 1.26 (d, J=7.2 Hz, 3H), 1.74 (m, 1H), 1.86 (m, 1H), 2.00 (m, 2H), 3.22 (d, half of AB quartet, J=21.9 Hz, 1H), 3.39 (d, half of AB quartet, J=21.9 Hz, 1H) 3.53 (m, 1H), 4.07 (m, 1H), 4.39 m, 1H), 5.04 (m, 2H), 6.88 (m, 1H), 6.93 (br d, J=7.8 Hz, 1H) 7.17 (m, 1H), 7.32 (m, 5H), 7.44 (ddd, J=8.3, 6.3 Hz, 1H).

Step 6. Synthesis of benzyl (5R,7S)-1-(3-fluorophenyl)-4-hydroxy-7-methyl-2-oxo-1,8-diazaspiro[4.5]decane-8-carboxylate (C16). A solution of C15 (881 mg, 2.15 mmol) in methanol (25 mL) and tetrahydrofuran (5 mL) at 0° C. was treated portion-wise with sodium borohydride (98%, 248 mg, 6.42 mmol), and the resulting yellow solution was stirred at 0° C. for 2 hours. Water (5 mL) was added, volatiles were removed in vacuo, and the remaining mixture was acidified to pH 3 with 1 N aqueous hydrochloric acid, then extracted with ethyl acetate (3×5 mL). The combined organic layers were dried over sodium sulfate, filtered and concentrated; the residue was purified via silica gel chromatography (Eluant: ethyl acetate) to afford C16 as a light brown foam. Yield 620 mg, 1.50 mmol, 70%. LCMS m/z 413.2 (M+1). ¹H NMR (400 MHz, CDCl₃) Mixture of two diastereomers, selected peaks: δ 1.18 and 1.21 (2 doublets, J=7.0, 7.2 Hz, 3H), 1.36 (m, <1H), 1.90 (m, <1H), 2.07 and 2.18 (2 broad doublets, J=13.1, 11.3 Hz, 1H), 2.37 (m, 1H), 2.86 (m, 1H), 3.03 (m, 1H), 5.03 (m, 2H), 6.80 (m, 2H), 7.06 (m, 1H), 7.29 (m, 6H).

Step 7. Synthesis of benzyl (5R,7S)-1-(3-fluorophenyl)-7-methyl-2-oxo-1,8-diazaspiro[4.5]dec-3-ene-8-carboxylate (C17). A solution of C16 (510 mg, 1.24 mmol) in pyridine (8.83 mL) was cooled to 0° C. and treated with thionyl chloride (0.270 mL, 3.71 mmol). The reaction was stirred for 1 hour at room temperature, then at 50° C. for 18 hours. After cooling to room temperature, volatiles were removed under reduced pressure, and the residue was dissolved in ethyl acetate and neutralized by repeated washing with an aqueous solution of sodium bicarbonate (4×10 mL). The organic layer was concentrated in vacuo and purified by chromatography on silica gel (Gradient: 20%-100% ethyl acetate in heptane), providing C17, Yield: 300 mg, 0.76 mmol, 61%. LCMS m/z 395.5 (M+1). ¹H NMR (500 MHz, CDCl₂) δ 1.32 (d, J=7.1 Hz, 3H), 1.50 (br d, J=12.9 Hz, 1H), 1.68 (m, 1H), 1.93 (m, 1H), 2.11 (m, 1H), 3.14 (m, 1H), 4.24 (m, 1H), 4.64 (m, 1H), 5.09 (m, 2H), 6.34 (d, J=6.1 Hz, 1H), 6.85 (m, 1H), 6.91 (br d, J=7.8 Hz, 1H), 7.14 (m, 1H), 7.30-7.37 (m, 5H), 7.43 (ddd. J=8.2, 8.2, 6.4 Hz, 1H), 7.64 (d, J=6.3 Hz, 1H).

Step 8. Synthesis of (5R,7S)-1-(3-fluorophenyl)-7-methyl-1,8-diazaspiro[4,5]dec˜3˜en˜2˜one (P3). Compound C17 (150 mg, 0.38 mmol) was dissolved in methanol (0.19 mL) and 6 N aqueous hydrochloric acid (1.27 ml., 7.6 mmol), and the reaction was heated at reflux for 2 hours. The mixture was concentrated in vacuo to one-half its original volume, and then extracted with ethyl acetate; this extract was discarded. The aqueous layer was cooled to 10° C., basified to pH 11 with 1 N aqueous sodium hydroxide solution, and extracted with ethyl acetate (3×10 mL). The combined organic layers were concentrated under reduced pressure to give P3 as an oil. Yield 32 mg, 0.12 mmol, 32%. LCMS m/z 261.2 (M+1). ¹H NMR (400 MHz, CDCl₃) δ 1.02 (d, J=6.5 Hz, 3H), 1.62 (dd, J=14.1, 9.9 Hz, 1H), 1.82˜1.90 (m, 2H), 1.96 (ddd, J=14.1, 10.9, 4.9 Hz, 1H), 2.63 (ddd, J=12.7, 10.9, 3,3 Hz, 1H), 2.73 (m, 1H), 2.89 (ddd, J=12.6, 4.6, 4.6 Hz, 1H), 6.16 (d, J=5.9 Hz, 1H), 6.93 (m, 1H), 6.99 (m. 1H), 7.03 (d, J=6.0 Hz, 1H), 7.12 (m, 1H), 7.41 (ddd, J=8.0, 8.0, 6.4 Hz, 1H).

### Preparation 4

### (5R,7S)-1-(3-fluorophenyl)-7-methyl-1,8-diazaspiro[4.5]decan-2-one (P4)

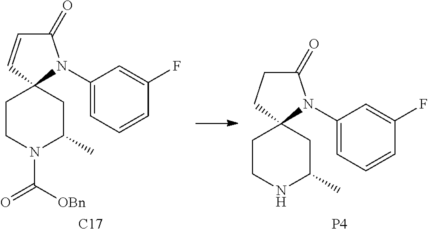

Synthesis of P4. Compound C17 (150 mg, 0.38 mmol) and palladium hydroxide (20% by weight on carbon, 26.7 mg, 0.038 mmol) were combined in methanol (4.75 mL) and hydrogenated for 18 hours under 45 psi of hydrogen. The reaction mixture was filtered through an Acrodisc® syringe filter and the filtrate was concentrated in vacuo to afford P4 as an oil. Yield: 70 mg, 0.27 mmol, 71%. LCMS m/z 263.5 (M+1). ¹H NMR (400 MHz, CDCl₃) δ 1.08 (d, J=6.4 Hz, 3H), 1.59 (dd, J=14.2, 9.2 Hz, 1H), 1.86 (ddd, J=14.3, 10.2, 4.5 Hz, 1H), 1.98˜2.14 (m, 4H), 2.50˜2.58 (m, 3H), 2.76 (m, 1H), 2.88 (ddd, J=13.0, 4.9, 4.9 Hz, 1H), 6.84 (ddd, J=9.3, 2.2, 2.2 Hz, 1H), 6.90 (m, 1H), 7.06 (dddd, J=8.3, 8.3, 2.4, 0.9 Hz, 1H), 7.35 (ddd, J=8.2, 8.2, 6.4 Hz, 1H).

### Examples 1-86

### Racemic 8˜substituted (5R,7S)(5S,7R)˜1˜(3˜fluorophenyl)˜7˜methyl˜1,8˜diazaspiro[4.5]dec-3-en-2-ones and 8-substituted (5R,7S)(5S,7R)-1-(3-fluorophenyl)-7-methyl-1,8-diazaspiro[4.5]decan-2-ones

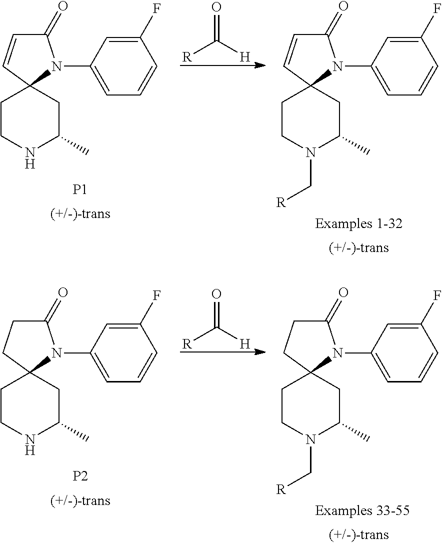

Synthesis of Examples 1-86. A solution of either compound P1 or P2 (0.19 M in dichloroethane, 400 μL, 75 μmol) was placed in an 8 mL vial, and treated with the aldehyde component (0.25 M solution in dichloroethane, 300 μL, 75 μmol). Sodium triacetoxyborohydride (225 μmol) was added to each vial, which was then capped and shaken at 30° C. for 16 hours. Solvent was removed using a SpeedVac system, and the crude products were purified by preparative HPLC. See Table 1 for characterization data.

Table 1 shows the structure of the compounds and relevant biological data that were measured in each case either on the compound as a free base or on the pharmaceutically acceptable salt of the compound disclosed in the Table. Each assay is disclosed in greater detail hereinbelow.

TABLE 1

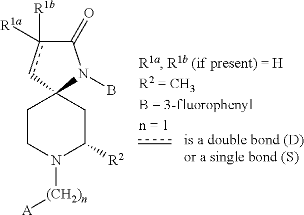

| Ex # | A | 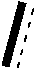 | BACE Activity¹ | IUPAC Name | Calc'd. Exact Mol. Wt. | Mass. Spec. (M + 1) | HPLC RT (min)² |
|---|---|---|---|---|---|---|---|
| 1 | 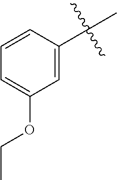 | D | ** | Racemic (5R,7S),(5S,7R)-8-(3- ethoxybenzyl)-1-(3-fluorophenyl)- 7-methyl-1,8-diazaspiro[4.5]dec- 3-en-2-one | 394.2 | 395 | 2.066³ |
| 2 | 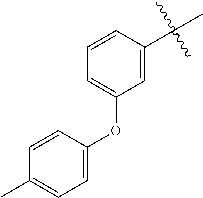 | D | *** | Racemic (5R,7S),(5S,7R)-1-(3- fluorophenyl)-7-methyl-8-[3-(4- methylphenoxy)benzyl]-1,8- diazaspiro[4.5]dec-3-en-2-one | 456.2 | 457 | 2.583³ |
| 3 | 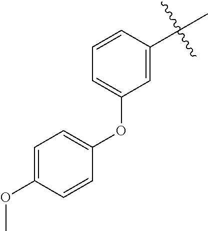 | D | ** | Racemic (5R,7S),(5S,7R)-1-(3- fluorophenyl)-8-[3-(4- methoxyphenoxy)benzyl]-7- methyl-1,8-diazaspiro[4.5]dec-3- en-2-one | 472.2 | 473 | 2.456³ |
| 4 | 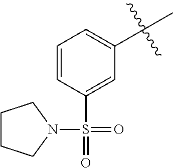 | D | ** | Racemic (5R,7S),(5S,7R)-1-(3- fluorophenyl)-7-methyl-8-[3- (pyrrolidin-1-ylsulfonyl)benzyl]- 1,8-diazaspiro[4.5]dec-3-en-2- one | 483.2 | 484 | 2.003³ |
| 5 | 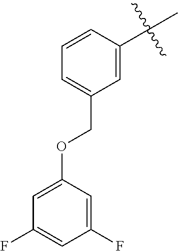 | D | ** | Racemic (5R,7S),(5S,7R)- 8-{3-[(3,5- difluorophenoxy)methyl]benzyl}- 1-(3-fluorophenyl)-7-methyl-1,8- diazaspiro[4.5]dec-3-en-2-one | 492.2 | 493 | 1.901⁴ |
| 6 | 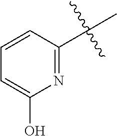 | D | * | Racemic (5R,7S),(5S,7R)-1-(3- fluorophenyl)-8-[(6- hydroxypyridin-2-yl)methyl]-7- methyl-1,8-diazaspiro[4.5]dec-3- en-2-one | 367.2 | 368 | 1.595⁵ |
| 7 | 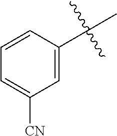 | D | ** | Racemic 3-{[(5R,7S),(5S,7R)-1- (3-fluorophenyl)-7-methyl-2-oxo- 1,8-diazaspiro[4.5]dec-3-en-8- yl]methyl}benzonitrile | 375.2 | 376 | 0.950³ |
| 8 | 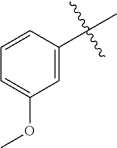 | D | * | Racemic (5R,7S),(5S,7R)-1-(3- fluorophenyl)-8-(3- methoxybenzyl)-7-methyl-1,8- diazaspiro[4.5]dec-3-en-2-one | 380.2 | 381 | 1.796³ |
| 9 | 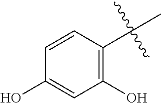 | D | * | Racemic (5R,7S),(5S,7R)-8-(2,4- dihydroxybenzyl)-1-(3- fluorophenyl)-7-methyl-1,8- diazaspiro[4.5]dec-3-en-2-one | 382.2 | 383 | 1.814⁵ |
| 10 | 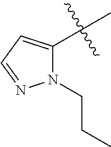 | D | ** | Racemic (5R,7S),(5S,7R)-1-(3- fluorophenyl)-7-methyl-8-[(1- propyl-1H-pyrazol-5-yl)methyl]- 1,8-diazaspiro[4.5]dec-3-en-2- one | 382.2 | 383 | 0.977³ |
| 11 | 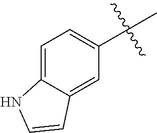 | D | ** | Racemic (5R,7S),(5S,7R)-1-(3- fluorophenyl)-8-(1H-indol-5- ylmethyl)-7-methyl-1,8- diazaspiro[4.5]dec-3-en-2-one | 389.2 | 390 | 1.669³ |
| 12 | 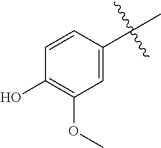 | D | *** | Racemic (5R,7S),(5S,7R)-1-(3- fluorophenyl)-8-(4-hydroxy-3- methoxybenzyl)-7-methyl-1,8- diazaspiro[4.5]dec-3-en-2-one | 396.2 | 397 | 1.813⁵ |
| 13 | 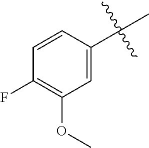 | D | ** | Racemic (5R,7S),(5S,7R)-8-(4- fluoro-3-methoxybenzyl)-1-(3- fluorophenyl)-7-methyl-1,8- diazaspiro[4.5]dec-3-en-2-one | 398.2 | 399 | 1.845³ |
| 14 | 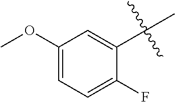 | D | * | Racemic (5R,7S),(5S,7R)-8-(2- fluoro-5-methoxybenzyl)-1-(3- fluorophenyl)-7-methyl-1,8- diazaspiro[4.5]dec-3-en-2-one | 398.2 | 399 | 1.859³ |
| 15 | 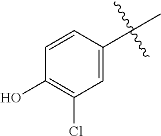 | D | *** | Racemic (5R,7S),(5S,7R)-8-(3- chloro-4-hydroxybenzyl)-1-(3- fluorophenyl)-7-methyl-1,8- diazaspiro[4.5]dec-3-en-2-one | 400.1 | 401 | 0.938³ |
| 16 | 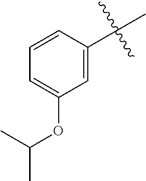 | D | *** | Racemic (5R,7S),(5S,7R)-1(3- fluorophenyl)-8-(3- isopropoxybenzyl)-7-methyl-1,8- diazaspiro[4.5]dec-3-en-2-one | 408.2 | 409 | 2.226³ |
| 17 | 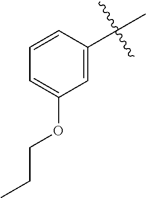 | D | ** | Racemic (5R,7S),(5S,7R)-1-(3- fluorophenyl)-7-methyl-8-(3- propoxybenzyl)-1,8- diazaspiro[4.5]dec-3-en-2-one | 408.2 | 409 | 2.300³ |
| 18 | 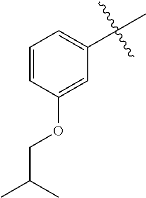 | D | ** | Racemic (5R,7S),(5S,7R)-1-(3- fluorophenyl)-8-(3- isobutoxybenzyl)-7-rnethyl-1,8- diazaspiro[4.5]dec-3-en-2-one | 422.2 | 423 | 2.483³ |
| 19 | 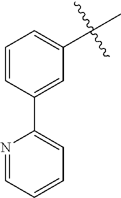 | D | ** | Racemic (5R,7S),(5S,7R)-1-(3- fluorophenyl)-7-methyl-8-(3- pyridin-2-ylbenzyl)-1,8- diazaspiro[4.5]dec-3-en-2-one | 427.2 | 428 | 1.970⁵ |
| 20 | 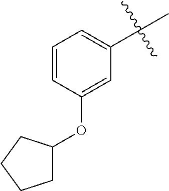 | D | ** | Racemic (5R,7S),(5S,7R)-8-[3- (cyclopentyloxy)benzyl]-1-(3- fluorophenyl)-7-methyl-1,8- diazaspiro[4.5]dec-3-en-2-one | 434.2 | 435 | 2.470³ |
| 21 | 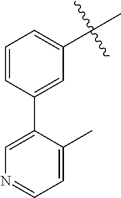 | D | *** | Racemic (5R,7S),(5S,7R)-1-(3- fluorophenyl)-7-methyl-8-[3-(4- methylpyridin-3-yl)benzyl]-1,8- diazaspiro[4.5]dec-3-en-2-one | 441.2 | 442 | 1.783⁵ |
| 22 | 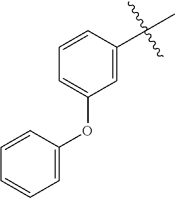 | D | ** | Racemic (5R,7S),(5S,7R)-1-(3- fluorophenyl)-7-methyl-8-(3- phenoxybenzyl)-1,8- diazaspiro[4.5]dec-3-en-2-one | 442.2 | 443 | 1.530⁴ |
| 23 | 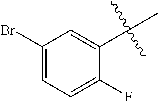 | D | ** | Racemic (5R,7S),(5S,7R)-8-(5- bromo-2-fluorobenzyl)-1-(3- fluorophenyl)-7-methyl-1,8- diazaspiro[4.5]dec-3-en-2-one | 446.1 | 447 | 2.009³ |
| 24 | 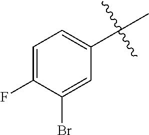 | D | * | Racemic (5R,7S),(5S,7R)-8-(3- bromo-4-fluorobenzyl)-1-(3- fluorophenyl)-7-methyl-1,8- diazaspiro[4.5]dec-3-en-2-one | 446.1 | 447 | 2.102³ |
| 25 | 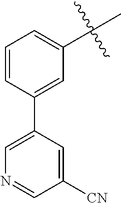 | D | ** | Racemic 5-(3-{[(5R,7S),(5S,7R)- 1-(3-fluorophenyl)-7-methyl-2- oxo-1,8-diazaspiro[4.5]dec-3-en- 8-yl]methyl}phenyl)nicotinonitrile | 452.2 | 453 | 1.981³ |
| 26 | 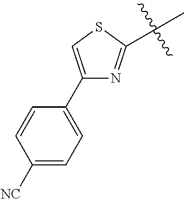 | D | ** | Racemic 4-(2-{[(5R,7S),(5S,7R)- 1-(3-fluorophenyl)-7-methyl-2- oxo-1,8-diazaspiro[4.5]dec-3-en- 8-yl]methyl}-1,3-thiazol-4- yl)benzonitrile | 458.2 | 459 | 2.154³ |
| 27 | 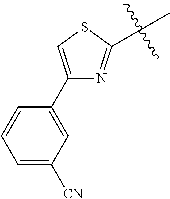 | D | ** | Racemic 3-(2-{[(5R,7S),(5S,7R)- 1-(3-fluorophenyl)-7-methyl-2- oxo-1,8-diazaspiro[4.5]dec-3-en- 8-yl]methyl}-1,3-thiazol-4- yl)benzonitrite | 458.2 | 459 | 2.173³ |
| 28 | 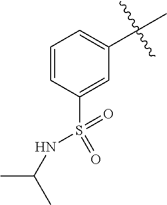 | D | ** | Racemic 3-{[(5R,7S),(5S,7R)-1- (3-fluorophenyl)-7-methyl-2-oxo- 1,8-diazaspiro[4.5]dec-3-en-8- yl]methyl}-N- isopropylbenzenesulfonamide | 471.2 | 472 | 1.852³ |
| 29 | 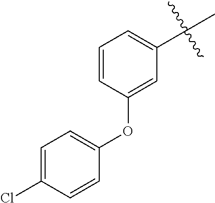 | D | ** | Racemic (5R,7S),(5S,7R)-8-[3- (4-chlorophenoxy)benzyl]-1-(3- fluorophenyl)-7-methyl-1,8- diazaspiro[4.5]dec-3-en-2-one | 476.2 | 477 | 3.586⁶ |
| 30 | 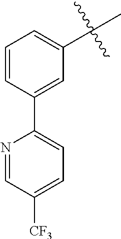 | D | * | Racemic (5R,7S),(5S,7R)-1-(3- fluorophenyl)-7-methyl-8-{3-[5- (trifluoromethyl)pyridin-2- yl]benzyl}-1,8-diazaspiro[4.5]dec- 3-en-2-one | 495.2 | 496 | 2.457³ |
| 31 | 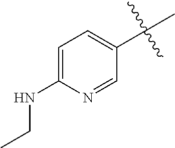 | D | * | Racemic (5R,7S),(5S,7R)-8-{[6- (ethylamino)pyridin-3-yl]methyl}- 1-(3-fluorophenyl)-7-methyl-1,8- diazaspiro[4.5]dec-3-en-2-one | 394.2 | 395 | 2.520⁶ |
| 32 | 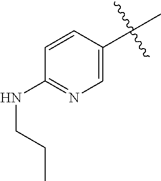 | D | * | Racemic (5R,7S),(5S,7R)-1-(3- fluorophenyl)-7-methyl-8-{[6- (propylamino)pyridin-3- yl]methyl}-1,8-diazaspiro[4.5]dec- 3-en-2-one | 408.2 | 409 | 1.610⁵ |
| 33 | 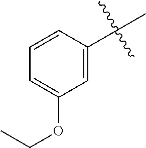 | S | * | Racemic (5R,7S),(5S,7R)-8-(3- ethoxybenzyl)-1-(3-fluorophenyl)- 7-methyl-1,8- diazaspiro[4.5]decan-2-one | 396.2 | 397 | 2.067³ |
| 34 | 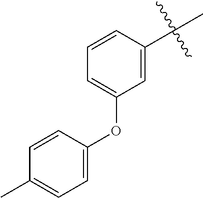 | S | ** | Racemic (5R,7S),(5S,7R)-1-(3- fluorophenyl)-7-methyl-8-[3-(4- methylphenoxy)benzyl]-1,8- diazaspiro[4.5]decan-2-one | 458.2 | 459 | 2.582³ |
| 35 | 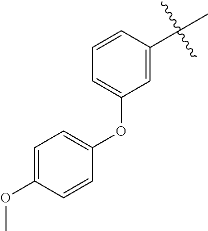 | S | ** | Racemic (5R,7S),(5S,7R)-1-(3- fluorophenyl)-8-[3-(4- methoxyphenoxy)benzyl]-7- methyl-1,8-diazaspiro[4.5]decan- 2-one | 474.2 | 475 | 3.413⁶ |
| 36 | 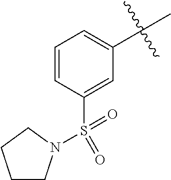 | S | * | Racemic (5R,7S),(5S,7R)-1-(3- fluorophenyl)-7-methyl-8-[3- (pyrrolidin-1-ylsulfonyl)benzyl]- 1,8-diazaspiro[4.5]decan-2-one | 485.2 | 486 | 1.961³ |
| 37 | 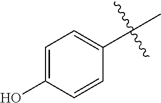 | S | ** | Racemic (5R,7S),(5S,7R)-1-(3- fluorophenyl)-8-(4- hydroxybenzyl)-7-methyl-1,8- diazaspiro[4.5]decan-2-one | 368.2 | 369 | 2.485⁶ |
| 38 | 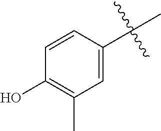 | S | ** | Racemic (5R,7S),(5S,7R)-1-(3- fluorophenyl)-8-(4-hydroxy-3- methylbenzyl)-7-methyl-1,8- diazaspiro[4.5]decan-2-one | 382.2 | 383 | 1.001³ |
| 39 | 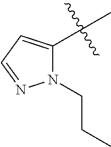 | S | * | Racemic (5R,7S),(5S,7R)-1-(3- fluorophenyl)-7-methyl-8-[(1- propyl-1H-pyrazol-5-yl)methyl]- 1,8-diazaspiro[4.5]decan-2-one | 384.2 | 385 | 1.943⁵ |
| 40 | 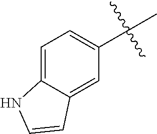 | S | * | Racemic (5R,7S),(5S,7R)-1-(3- fluorophenyl)-8-(1H-indol-5- ylmethyl)-7-methyl-1,8- diazaspiro[4.5]decan-2-one | 391.2 | 392 | 1.685³ |
| 41 | 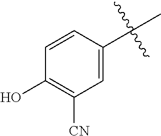 | S | ** | Racemic (5R,7S),(5S,7R)-1(3- fluorophenyl)-7-methyl-2-oxo-1,8- diazaspiro[4.5]dec-8-yl]methyl}-2- hydroxybenzonitrile | 393.2 | 394 | 1.780⁵ |
| 42 | 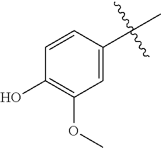 | S | ** | Racemic (5R,7S),(5S,7R)-1-(3- fluorophenyl)-8-(4-hydroxy-3- methoxybenzyl)-7-methyl-1,8- diazaspiro[4.5]decan-2-one | 398.2 | 399 | 2.684⁷ |
| 43 | 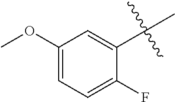 | S | * | Racemic (5R,7S),(5S,7R)-8-(2- fluoro-5-methoxybenzyl)-1-(3- fluorophenyl)-7-methyl-1,8- diazaspiro[4.5]decan-2-one | 400.2 | 401 | 1.861³ |
| 44 | 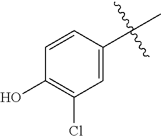 | S | ** | Racemic (5R,7S),(5S,7R)-8-(3- chloro-4-hydroxybenzyl)-1-(3- fluorophenyl)-7-methyl-1,8- diazaspiro[4.5]decan-2-one | 402.2 | 403 | 0.957³ |
| 45 | 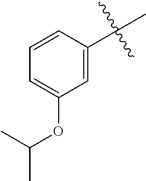 | S | ** | Racemic (5R,7S),(5S,7R)-1-(3- fluorophenyl)-8-(3- isopropoxybenzyl)-7-methyl-1,8- diazaspiro[4.5]decan-2-one | 410.2 | 411 | 2.222³ |
| 46 | 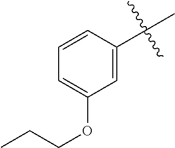 | S | ** | Racemic (5R,7S),(5S,7R)-1-(3- fluorophenyl)-7-methyl-8-(3- propoxybenzyl)-1,8- diazaspiro[4.5]decan-2-one | 410.2 | 411 | 2.298³ |
| 47 | 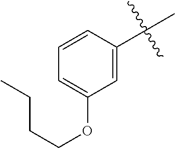 | S | * | Racemic (5R,7S),(5S,7R)-8-(3- butoxybenzyl)-1-(3-fluorophenyl)- 7-methyl-1,8- diazaspiro[4.5]decan-2-one | 424.3 | 425 | 2.478³ |
| 48 | 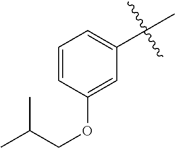 | S | * | Racemic (5R,7S),(5S,7R)-1-(3- fluorophenyl)-8-(3- isobutoxybenzyl)-7-methyl-1,8- diazaspiro[4.5]decan-2-one | 424.3 | 425 | 3.539⁶ |
| 49 | 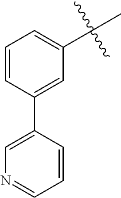 | S | * | Racemic (5R,7S),(5S,7R)-1-(3- fluorophenyl)-7-methyl-8-(3- pyridin-3-ylbenzyl)-1,8- diazaspiro[4.5]decan-2-one | 429.2 | 430 | 2.888⁶ |
| 50 | 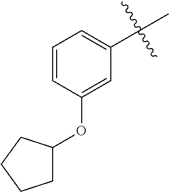 | S | * | Racemic (5R,7S),(5S,7R)-8-[3- (cyclopentyloxy)benzyl]-1-(3- fluorophenyl)-7-methyl-1,8- diazaspiro[4.5]decan-2-one | 436.3 | 437 | 2.468³ |
| 51 | 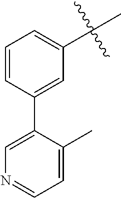 | S | ** | Racemic (5R,7S),(5S,7R)-1-(3- fluorophenyl)-7-methyl-8-[3-(4- methylpyridin-3-yl)benzyl]-1,8- diazaspiro[4.5]decan-2-one | 443.2 | 444 | 1.808⁵ |
| 52 | 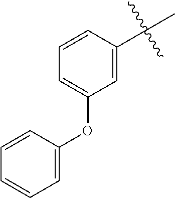 | S | ** | Racemic (5R,7S),(5S,7R)-1-(3- fluorophenyl)-7-methyl-8-(3- phenoxybenzyl)-1,8- diazaspiro[4.5]decan-2-one | 444.2 | 445 | 2.577⁵ |
| 53 | 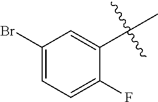 | S | * | Racemic (5R,7S),(5S,7R)-8-(5- bromo-2-fluorobenzyl)-1-(3- fluorophenyl)-7-methyl-1,8- diazaspiro[4.5]decan-2-one | 448.1 | 449 | 2.288⁵ |
| 54 | 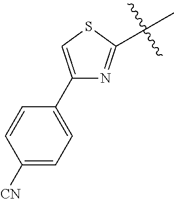 | S | * | Racemic 4-(2-{[(5R,7S),(5S,7R)- 1-(3-fluorophenyl)-7-methyl-2- oxo-1,8-diazaspiro[4.5]dec-8- yl]methyl}-1,3-thiazol-4- yl)benzonitrile | 460.2 | 461 | 2.124³ |
| 55 | 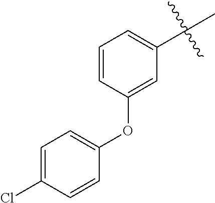 | S | ** | Racemic (5R,7S),(5S,7R)-8-[3- (4-chlorophenoxy)benzyl]-1-(3- fluorophenyl)-7-methyl-1,8- diazaspiro[4.5]decan-2-one | 478.2 | 479 | 2.615³ |
| 59 | 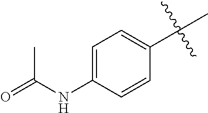 | D | *** | Racemic N-(4-{[(5R,7S),(5S,7R)- 1-(3-fluorophenyl)-7-methyl-2- oxo-1,8-diazaspiro[4.5]-3-en 8-yl]methyl}phenyl)acetamide | 407.2 | 408 | 1.978⁵ |
| 60 | 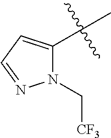 | D | ** | Racemic (5R,7S),(5S,7R)-1-(3- fluorophenyl)-7-methyl-8-{[1- (2,2,2-trifluoroethyl)-1H-pyrazol- 5-yl]methyl}-1,8- diazaspiro[4.5]dec-3-en-2-one | 422.2 | 423 | 2.225⁵ |
| 61 | 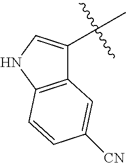 | D | ** | Racemic 3-{[(5R,7S),(5S,7R)-1- (3-fluorophenyl)-7-methyl-2-oxo- 1,8-diazaspiro[4.5]dec-3-en-8- yl]methyl}-1H-indole-5- carbonitrile | 414.2 | 415 | 1.797³ |
| 62 | 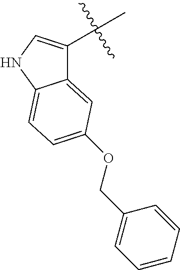 | D | ** | Racemic (5R,7S),(5S,7R)-8-{[5- (benzyloxy)-1H-indol-3- yl]methyl}-1-(3-fluorophenyl)-7- methyl-1,8-diazaspiro[4.5]dec-3- en-2-one | 495.2 | 496 | 1.723⁴ |
| 63 | 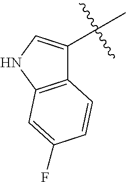 | D | ** | Racemic (5R,7S),(5S,7R)-8-[(6- fluoro-1H-indol-3-yl)methyl]-1-(3- fluorophenyl)-7-methyl-1,8- diazaspiro[4.5]dec-3-en-2-one | 407.2 | 408 | 2.024³ |
| 64 | 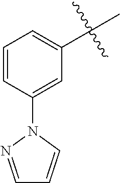 | D | ** | Racemic (5R,7S),(5S,7R)-1-(3- fluorophenyl)-7-methyl-8-[3-(1H- pyrazol-1-yl)benzyl]-1,8- diazaspiro[4.5]dec-3-en-2-one | 416.2 | 417 | 1.985³ |
| 65 | 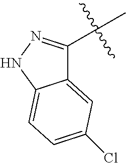 | D | ** | Racemic (5R,7S),(5S,7R)-8-[(5- chloro-1H-indazol-3-yl)methyl]-1- (3-fluorophenyl)-7-methyl-1,8- diazaspiro[4.5]dec-3-en-2-one | 424.1 | 425 | 2.038³ |
| 66 | 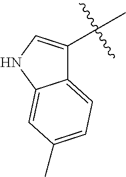 | D | ** | Racemic (5R,7S),(5S,7R)-1-(3- fluorophenyl)-7-methyl-8-[(6- methyl-1H-indol-3-yl)methyl]-1,8- diazaspiro[4.5]dec-3-en-2-one | 403.2 | 404 | 2.163³ |
| 67 | 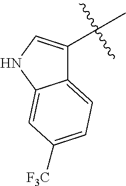 | D | ** | Racemic (5R,7S),(5S,7R)-1-(3- fluorophenyl)-7-methyl-8-{[6- (trifluoromethyl)-1H-indol-3- yl]methyl}-1,8-diazaspiro [4.5]dec-3-en-2-one | 457.2 | 458 | 2.501⁵ |
| 68 | 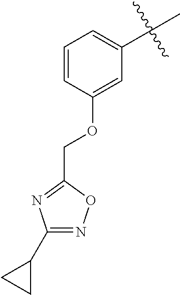 | D | ** | Racemic (5R,7S),(5S,7R)-8-{3- [(3-cyclopropyl-1,2,4-oxadiazol-5- yl)methoxy]benzyl}-1-(3- fluorophenyl)-7-methyl-1,8- diazaspiro[4.5]dec-3-en-2-one | 488.2 | 489 | 2.316³ |
| 69 | 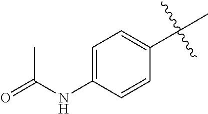 | S | ** | Racemic N-(4-{[(5R,7S),(5S,7R)- 1-(3-fluorophenyl)-7-methyl-2- oxo-1,8-diazaspiro[4.5]dec-8- yl]methyl}phenyl)acetamide | 409.2 | 410 | 1.964⁵ |
| 70 | 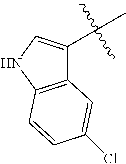 | S | ** | Racemic (5R,7S),(5S,7R)-8-[(5- chloro-1H-indol-3-yl)methyl]-1-(3- fluorophenyl)-7-methyl-1,8- diazaspiro[4.5]decan-2-one | 425.2 | 426 | 2.185³ |
| 71 | 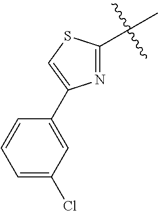 | D | ** | Racemic (5R,7S),(5S,7R)-8-{[4- (3-chlorophenyl)-1,3-thiazol-2- yl]methyl}-1-(3-fluorophenyl)-7- methyl-1,8-diazaspiro[4.5]dec-3- en-2-one | 467.1 | 468 | 1.864⁴ |
| 72 | 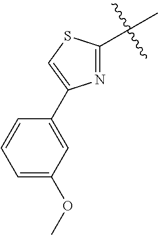 | D | ** | Racemic (5R,7S),(5S,7R)-1-(3- fluorophenyl)-8-{[4-(3- methoxyphenyl)-1,3-thiazol-2- yl]methyl}-7-methyl-1,8- diazaspiro[4.5]dec-3-en-2-one | 463.2 | 464 | 2.36³ |
| 73 | 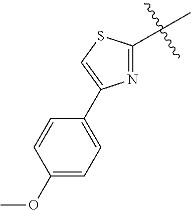 | D | ** | Racemic (5R,7S),(5S,7R)-1-(3- fluorophenyl)-8-{[4-(4- methoxyphenyl)-1,3-thiazol-2- yl]methyl}-7-methyl-1,8- diazaspiro[4.5]dec-3-en-2-one | 463.2 | 464 | 2.335³ |
| 74 | 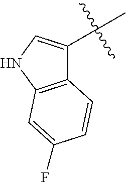 | S | * | Racemic (5R,7S),(5S,7R)-8-[(6- fluoro-1H-indol-3-yl)methyl]-1-(3- fluorophenyl)-7-methyl-1,8- diazaspiro[4.5]decan-2-one | 409.2 | 410 | 2.019³ |
| 75 | 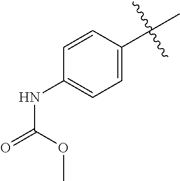 | S | * | Racemic methyl (4- {[(5R,7S),(5S,7R)-1-(3- fluorophenyl)-7-methyl-2-oxo-1,8- diazaspiro[4.5]dec-8- yl]methyl}phenyl)carbamate | 425.2 | 426 | 1.767³ |
| 76 | 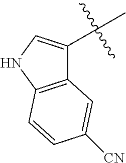 | S | * | Racemic 3-{[(5R,7S),(5S,7R)-1- (3-fluorophenyl)-7-methyl-2-oxo- 1,8-diazaspiro[4.5]dec-8- yl]methyl}-1H-indole-5- carbonitrile | 416.2 | 417 | 1.793³ |
| 77 | 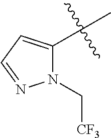 | S | * | Racemic (5R,7S),(5S,7R)-1-(3- fluorophenyl)-7-methyl-8-{[1- (2,2,2-trifluoroethyl)-1H-pyrazol- 5-yl]methyl}-1,8- diazaspiro[4.5]decan-2-one | 424.2 | 425 | 2.157⁵ |
| 78 | 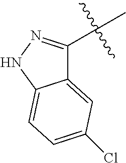 | S | * | Racemic (5R,7S),(5S,7R)-8-[(5- chloro-1H-indazol-3-yl)methyl]-1- (3-fluorophenyl)-7-methyl-1,8- diazaspiro[4.5]decan-2-one | 426.2 | 427 | 2.024³ |
| 79 | 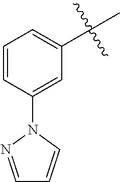 | S | * | Racemic (5R,7S),(5S,7R)-1-(3- fluorophenyl)-7-methyl-8-[3-(1H- pyrazol-1-yl)benzyl]-1,8- diazaspiro[4.5]decan-2-one | 418.2 | 419 | 1.982³ |
| 80 | 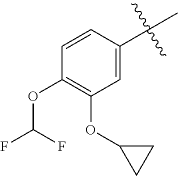 | D | * | Racemic (5R,7S),(5S,7R)-8-[3- (cyclopropyloxy)-4- (difluoromethoxy)benzyl]-1-(3- fluorophenyl)-7-methyl-1,8- diazaspiro[4.5]dec-3-en-2-one | 472.2 | 473 | 2.394³ |
| 81 | 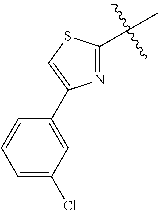 | S | * | Racemic (5R,7S),(5S,7R)-8-{[4- (3-chlorophenyl)-1,3-thiazol-2- yl]methyl}-1-(3-fluorophenyl)-7- methyl-1,8-diazaspiro[4.5]decan- 2-one | 469.1 | 470 | 1.779⁴ |
| 82 | 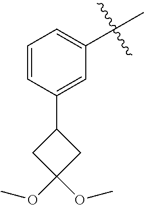 | D | * | Racemic (5R,7S),(5S,7R)-8-[3- (3,3-dimethoxycyclobutyl)benzyl]- 1-(3-fluorophenyl)-7-methyl-1,8- diazaspiro[4.5]dec-3-en-2-one | 464.2 | 465 | 2.288³ |
| 83 | 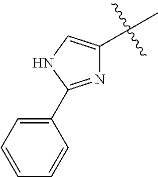 | D | * | Racemic (5R,7S),(5S,7R)-1-(3- fluorophenyl)-7-methyl-8-[(2- phenyl-1H-imidazol-4-yl)methyl]- 1,8-diazaspiro[4.5]dec-3-en-2- one | 416.2 | 417 | 1.747³ |
| 84 | 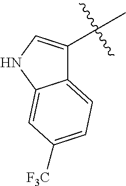 | S | * | Racemic (5R,7S),(5S.7R)-1-(3- fluorophenyl)-7-methyl-8-{[6- (trifluoromethyl)-1H-indol-3- yl]methyl}-1,8- diazaspiro[4.5]decan-2-one | 459.2 | 460 | 2.562⁵ |
| 85 | 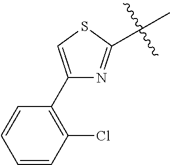 | D | * | Racemic (5R,7S),(5S,7R)-8-{[4- (2-chlorophenyl)-1,3-thiazol-2- yl]methyl}-1-(3-fluorophenyl)-7- methyl-1,8-diazaspiro[4.5]dec-3- en-2-one | 467.1 | 468 | 2.479³ |
| 86 | 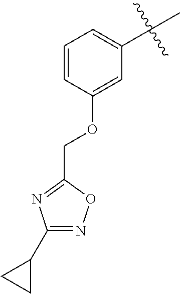 | S | * | Racemic (5R,7S),(5S,7R)-8-{3- [(3-cyclopropyl-1,2,4-oxadiazol-5- yl)methoxy]benzyl}-1-(3- fluorophenyl)-7-methyl-1,8- diazaspiro[4.5]decan-2-one | 490.2 | 491 | 2.307³ |

```
¹BACE activity Cell Free Assay IC₅₀; 1 nM to 1 μM ****, 1 μM to 10 μM ***, 10 μM to 100 μM**, 100 μM to 300 μM*
²HPLC conditions: Flow rate 0.8 mL/min; 50° C.; column and gradient described in footnote for each value.
³Column: Ymc ODS-AQ, 2.0 × 50 mm, 5 μm; Mobile phase A: 0.0375% TFA in water (v/v); Mobile phase B: 0.01875% TFA in acetonitrile (v/v);
Gradient:
  0 minutes  10% B
0.5 minutes  10% B
  4 minutes 100% B
4.3 minutes  10% B
4.7 minutes  10% B
⁴Column: Ymc ODS-AQ, 2.0 × 50 mm, 5 μm; Mobile phase A: 0.0375% TFA in water (v/v); Mobile phase B: 0.01875% TFA in acetonitrile (v/v);
Gradient:
  0 minutes  25% B
0.5 minutes  25% B
  4 minutes 100% B
4.3 minutes  25% B
4.7 minutes  25% B
⁵Column: Ymc ODS-AQ, 2.0 × 50 mm, 5 μm; Mobile phase A: 0.0375% TFA in water (v/v); Mobile phase B: 0.01875% TFA in acetonitrile (v/v);
Gradient:
  0 minutes  1% B
0.6 minutes  5% B
  4 minutes 100% B
4.3 minutes  1% B
4.7 minutes  1% B
⁶Column: Welch XB-C18, 2.1 × 50 mm, 5 μm; Mobile phase A: 0.05% NH₄OH in water (v/v); Mobile phase B: 100% acetonitrile;
Gradient:
  0 minutes  5% B
  0.5 minutes  5% B
  3.4 minutes 100% B
  4.2 minutes 100% B
4.21 minutes  5% B
  4.7 minutes  5% B
⁷Column: Ymc ODS-AQ, 2.0 × 50 mm, 5 μm; Mobile phase A: 0.0375% TFA in water (v/v); Mobile phase B: 0.01875% TFA in acetonitrile (v/v);
Gradient:
  0 minutes  0% B
1.0 minutes  5% B
4.0 minutes 70% B
4.1 minutes  0% B
4.7 minutes  0% B
```

### Example 87

### (5R7S)-1-(3-fluorophenyl)-8-(4-hydroxy-3-isopropoxybenzyl)-7-methyl-1,8-diazaspiro[4,5]dec-3-en-2-one hydrochloride (87)

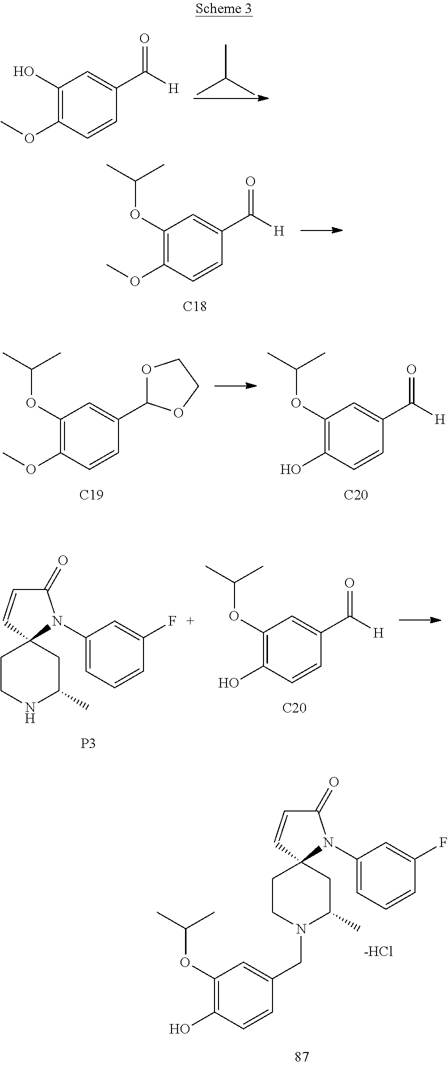

Step 1. Synthesis of 3-isopropoxy-4-methoxybenzaldehyde (C18). A solution of 3-hydroxy-4-methoxybenzaldehyde (5.00 g, 32.9 mmol) in dimethylformamide (100 mL) was treated with potassium carbonate (9.08 g, 65.7 mmol) and 2˜iodopropane (6.57 mL, 65.7 mmol). The reaction was stirred for 4 hours and then additional 2˜iodopropane (3.29 mL, 32.9 mmol) was added and the mixture was allowed to react for an additional hour. It was then poured into water and extracted with ethyl acetate (3×20 mL). The combined organic layers were washed with 1 N aqueous sodium hydroxide solution, then with saturated aqueous sodium chloride solution, dried, filtered and concentrated in vacuo to provide C18 as an oil. Yield: 4.60 g, 23.7 mmol, 72%. LCMS m/z 195.2 (M+1). ¹H NMR (400 MHz, CDCl₃) δ 1.41 (d, J=6.2 Hz, 6H), 3.95 (s, 3H), 4.65 (m, 1H), 6.99 (d, J=8.1 Hz, 1H), 7.42˜7.46 (m, 2H) 9.85 (s, 1H).

Step 2. Synthesis of 2˜(3˜isopropoxy˜4˜methoxyphenyl)˜1,3˜dioxolane (C19). Ethylene glycol (99%, 2.63 mL, 47.4 mmol) and para˜toluenesulfonic acid monohydrate (97%, 75 mg, 0.38 mmol) were added to a solution of C18 (4.6 g, 23.7 mmol) in toluene (79 mL). The reaction flask was equipped with a Dean-Stark trap, and the contents were heated at reflux for 5 hours. The reaction was poured into aqueous potassium carbonate solution, and the organic layer was then washed an additional two times with aqueous potassium carbonate solution, and once with saturated aqueous sodium chloride solution. The organic layer was dried, filtered and concentrated; NMR and LCMS revealed that the reaction was incomplete, so the product was resubjected to the reaction conditions, heating at reflux for 18 hours. The workup was repeated, to afford C19 as an oil. Yield: 5.0 g, 21.0 mmol, 89%. ¹H NMR (400 MHz, CDCl₃) δ 1.38 (d, J=6.2 Hz, 6H), 3.86 (s, 3H), 4.02 (m, 2H), 4.14 (m, 2H), 4.57 (septet, J=6.0 Hz, 1H), 5.75 (s, 1H), 6.88 (d, J=8.7 Hz, 1H), 7.03 (m, 2H),

Step 3. Synthesis of 4˜hydroxy˜3˜isopropoxybenzaldehyde (C20). Lithium wire (cut into small segments, 204 mg, 29.4 mmol) was added to a solution of chlorodiphenylphosphine (2.17 mL, 11.7 mmol) in tetrahydrofuran (18.7 mL), and the reaction was stirred for 1 hour. A solution of C19 (2.00 g, 8.39 mmol) in tetrahydrofuran (5 mL) was then added drop˜wise to the dark red mixture, and the reaction was stirred for 2 hours. It was then filtered into an aqueous sodium hydroxide solution, and extracted with diethyl ether (3×15 mL); the combined organic layers were washed with 1N aqueous sodium hydroxide solution, and the aqueous layers were combined and cooled in an ice bath. This aqueous phase was acidified with concentrated aqueous hydrochloric acid. The mixture was extracted with diethyl ether (3×10 mL) and these three organic layers were combined and washed with saturated aqueous sodium chloride solution, dried and concentrated in vacuo to give C20 as an oil. Yield: 740 mg, 4.11 mmol, 49%. ¹H NMR (400 MHz, CDCl₃) δ 1.41 (d, J=6.0 Hz, 6H), 4.73 (septet, J=6.1 Hz, 1H), 6.30 (s, 1H), 7.05 (d, J=8.0 Hz, 1H), 7.40 (m, 2H), 9.82 (5, 1H).

Step 4. Synthesis of 87. Compound C20 (20.7 mg, 0.115 mol) in dichloroethane (0.5 mL) was combined with a solution of P3 (20 mg, 0.077 mmol) in dichloroethane (0.4 mL). Acetic acid (4 μL, 0.07 mmol) was added. After 5 hours of stirring, the reaction was treated with sodium triacetoxyborohydride (32.6 mg, 0.154 mmol), and the reaction mixture was allowed to stir for 18 hours. Aqueous sodium bicarbonate solution was then added, and the layers were separated. The aqueous layer was extracted with dichloromethane (3×5 mL), and the combined organic layers were dried over sodium sulfate, filtered and concentrated in vacuo. The residue was purified by chromatography on silica gel (Gradient: 20%-70% ethyl acetate in heptane) to provide the free base of 87 as an oil. Yield: 7.8 mg, 0.018 mmol, 23%. LCMS m/z 425.2 (M+1). ¹H NMR (400 MHz, CDCl₃) δ 1.14 (d, J=6.8 Hz, 3H), 1.33 (2 overlapping doublets, J=6.0, 6.0 Hz, 6H), 1.59 (m, 1H), 1.71 (m, 1H), 1.97 (ddd, J=13.1, 9.8, 4.1 Hz, 1H), 2.11 (dd, J=13.3, 5.1 Hz, 1H), 2.41 (ddd, J=12.5, 5.3, 4.3 Hz, 1H), 2.64 (ddd, J=12.7, 9.8, 3.0 Hz, 1H), 2.99 (m, 1H), 3.35 (d, J=13.3 Hz, 1H), 3.55 (d, J=13.3 Hz, 1H), 4.52 (septet, J=6.0 Hz, 1H), 5.63 (br s, 1H), 6.23 (d, J=6.2 Hz, 1H), 6.68 (dd, J=8.0, 1.6 Hz, 1H), 6.77 (br s, 1H), 6.81 (d, J=8.0 Hz, 1H), 6.89 (ddd, J=9.5, 2.2, 2.2 Hz, 1H), 6.95 (br d, J=7.8 Hz, 1H), 7.13 (ddd, J=8.4, 8.4, 2.5 Hz, 1H), 7.38-7.44 (m, 2H). ¹³C NMR (100 MHz, CDCl₃). Not all of the expected signals were observed. δ 15.16, 22.06, 22.12, 33.63, 40.34, 43.96, 51.00, 57.80, 71.47, 113.62, 113.95, 115.60 (d, J=21 Hz), 118.12 (d, J=22 Hz), 121.48, 124.93, 126.60 (d, J=3 Hz). 130.38 (d, J=9 Hz), 144.41, 145.61, 153.60. The hydrochloride salt was prepared by dissolving the free base of 87 in diethyl ether and treating the solution with a 1.0 M solution of hydrochloric acid in ether, followed by concentration in vacuo. Compound 87 was obtained as a solid. Yield: 8.6 mg, 0.18 mmol, 100%.

### Example 88

### (5R,7S)˜1˜(3˜fluorophenyl)˜7˜methyl˜8˜[(2′˜methylbiphenyl˜3˜yl)methyl]˜1,8˜diazaspiro[4.5]decan˜2˜one hydrochloride (88)

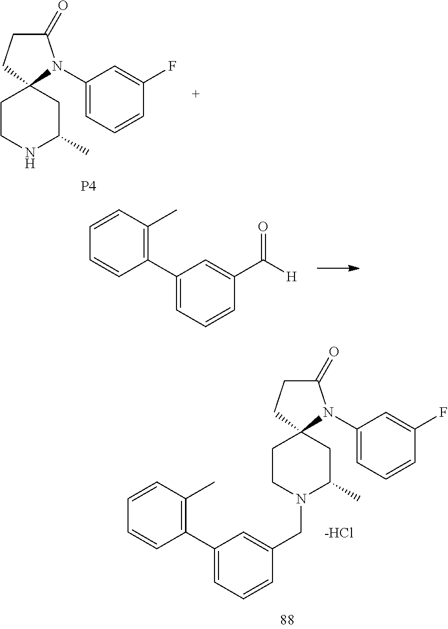

Compound 88 was prepared according to the general procedure for the synthesis of 87 in Example 87, except that P4 and 2′˜methylbiphenyl˜3˜carbaldehyde were used instead of P3 and C20, to provide the free base of 88 as an oil. Yield: 16.5 mg, 0.037 mmol, 48%. LCMS m/z 443.2 (M+1). ¹H NMR (500 MHz, CDCl₃) δ 1.14 (d, J=6.8 Hz, 3H), 1.60 (m, 1H), 1.70 (m, 1H), 1.89 (m, 1H), 2.04 (dd, J=13.2, 5.4 Hz, 1H), 2.13 (ddd, J=12.4, 9.5, 9.5 Hz, 1H), 2.25 (s, 3H), 2.31 (ddd, J=12.7, 8.8, 3.9 Hz, 1H), 2.49 (ddd, J=12.4, 4.4, 4.4 Hz, 1H), 2.53-2.69 (m, 3H), 3.04 (m, 1H), 3.51 (d, J=13.6 Hz, 1H), 3.63 (d, J=13.4 Hz, 1H), 6.87 (ddd, J=9.3, 2.1, 2.1 Hz, 1H), 6.92 (br d, J=8.3 Hz, 1H), 7.11 (ddd, J=8.4, 8.4, 2.4 Hz, 1H), 7.20˜7.28 (m, 7H), 7.33 (dd, J=7.6, 7.6 Hz, 1H), 7.40 (ddd, J=8.0, 8.0, 6.3 Hz, 1H). ¹³C NMR (125 MHz, CDCl₃) δ 13.79, 20.36, 29.80, 33.40, 34.42, 42.57, 43.63, 51.52, 58.13, 64.03, 115.45 (d, J=20 Hz), 117.90 (d, J=22 Hz), 125.69, 126.30 (d, J=3 Hz), 126.99, 127.15, 127.76, 127.89, 129.49, 129,68, 130.18 (d, J=9 Hz), 130.28, 135.17, 138.00, 138.08, 138.56, 141.71 (d, J=6 Hz), 162.76 (d, J=248 Hz), 175.01. The hydrochloride salt was prepared by dissolving the free base of 88 in diethyl ether and treating the solution with a 1.0 M solution of hydrochloric acid in ether, followed by concentration in vacuo. Compound 57 was obtained as a solid. Yield: 18 mg, 0.037 mmol, 100%.

### Example 89

### (5R,7S)˜1˜(3˜fluorophenyl)˜8˜(4˜hydroxy˜3˜isopropoxybenzyl)˜7˜methyl˜1,8˜diazaspiro[4.5]decan˜2˜one hydrochloride (89)

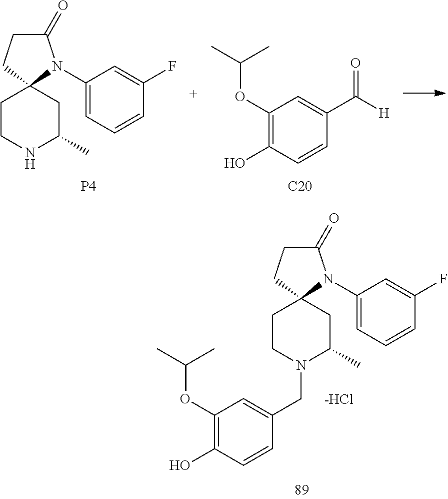

Synthesis of 89. Compound 89 was prepared according to the general procedure for the synthesis of 87 in Example 87, except that P4 was used instead of P3, to provide the free base of 89 as an oil. Yield: 26 mg, 0.060 mmol, 40%. LCMS m/z 427.1 (M+1). ¹H NMR (500 MHz, CDCl₃) δ 1.10 (d, J=6.8 Hz, 3H), 1.32 (d, J=6.1 Hz, 6H), 1.56 (m, 1H), 1.67 (m, 1H), 1.84 (m, 1H), 2.00 (dd, J=13.2, 5.4 Hz, 1H), 2.10 (ddd, J=12.4, 9.5, 9.5 Hz, 1H), 2.28 (ddd, J=12.7, 8.8, 3.9 Hz, 1H), 2.42 (ddd, J=12.4, 4.5, 4.5 Hz, 1H), 2.51-2.63 (m, 3H), 2.97 (m, 1H), 3.38 (d, J=13.2 Hz, 1H), 3.46 (d, J=13.2 Hz, 1H), 4.51 (septet, J=6.1 Hz, 1H), 6.67 (dd, J=7.9, 1.6 Hz, 1H), 6.77 (m, 1H), 6.80 (d, J=8.0 Hz, 1H), 6.85 (ddd, J=9.4, 2.1, 2.1 Hz, 1H), 6.90 (br d, J=7.8 Hz, 1H), 7.10 (ddd, J=8.4, 8.4, 2.5 Hz, 1H), 7.38 (ddd, J=8.0, 8.0, 6.4 Hz, 1H). ¹³C NMR (100 MHz, CDCl₃) Not all of the expected signals were observed. δ 13.70, 22.03, 22.11, 29.84, 33.45, 34.51, 42.54, 43.27, 51.27, 57.86, 64.12, 71.42, 113.54, 113.88, 115.38 (d, J=21 Hz), 117.91 (d, J=22 Hz), 121.36, 126.40 (d, J=3 Hz), 130.15 (d, J=9 Hz), 144.41, 145.52, 175.05. The hydrochloride salt was prepared by dissolving the free base of 89 in diethyl ether and treating the solution with a 1.0 M solution of hydrochloric acid in ether, followed by concentration in vacuo. Compound 89 was obtained as a solid. Yield: 28 mg, 0.060 mmol, 100%.

### Example 90 and 91

### Racemic (5R,7S)(5S,7R)-3-fluoro-1-(3-fluorophenyl)-8-(3-isopropoxybenzyl)-7˜methyl˜1,8˜diazaspiro[4.5]decan˜2˜one, formate salt (90) and Racemic (5R,7S)(5S,7R)˜3,3˜difluoro˜1˜(3˜fluorophenyl)˜8˜(3˜isopropoxybenzyl)˜7˜methyl˜1,8˜diazaspiro[4.5]decan˜2˜one, formate salt (91)

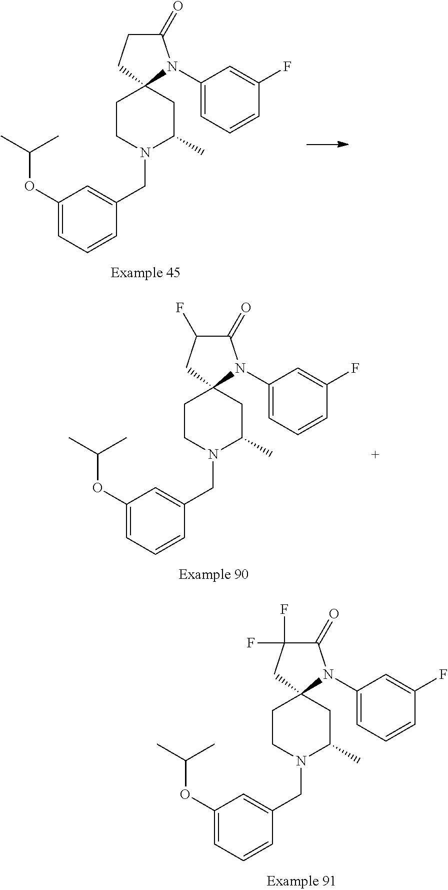

A flame˜dried flask under a nitrogen atmosphere was charged with dry THF (5 mL) and diisopropylamine (106 mg, 1.05 mmol) and was cooled to ˜78° C. in a dry ice-acetone bath, n-BuLi (0.37 mL, 0.93 mmol) was added dropwise, then the solution was warmed to −55° C. for 1 h and then cooled back to −78° C. A solution of Example 45 (240 mg, 0.58 mmol) in anhydrous THF (3 mL) was added dropwise, then the resulting mixture was stirred for 45 min at ˜78° C. and allowed to warm to ˜55° C. A solution of (PhSO₂)₂NF (276 mg, 0.87 mmol) in anhydrous THF (2 mL) was added dropwise, and the reaction mixture was stirred for 1 h at ˜55° C. The reaction was quenched with saturated NH₄Cl (10 mL) and the solvents were removed in vacuo. The residue was partitioned between EtOAc (10 mL) and water (10 mL). After separating layers, the aqueous layer was re˜extracted with EtOAc. The combined organic layers were dried over Na₂SO₄, and evaporated to give crude product, which was purified by preparative HPLC to obtain Example 90 (25 mg, 10%) as a white solid and Example 91 (58 mg, 22%) as a white solid.

### Example 90

¹H NMR (400 MHz, MeOD): δ 7.51˜7.45 (m, 1H), 7.23˜7.13 (m, 2H), 7.04˜6.99 (m, 2H), 6.78˜6.74 (d, 3H), 5.31˜5.15 (m, 1H), 4.56˜4.50 (s, 1H), 3.63-3.48 (m, 2H), 2.94˜2.47 (m, 5H), 2.11˜1.73 (d, 4H), 1.28˜1.21 (d, 6H), 1.22˜1.18 (t, 3H). HPLC Column YMC ODS˜AQ (0.46×5 cm×5 μm), RT=1.99 min, Mobile Phase 10% MeCN (0.1% TFA) in water to 80% MeCN (0.1% TFA) in water. LCMS m/z 429.4 (M+1).

### Example 91

¹H NMR (400 MHz, MeOD): δ 7.54˜7.49 (m, 1H), 7.28˜7.23 (m, 1H), 7 17˜7 13 (m, 1H), 7.09˜7.06 (d, 2H), 6.78˜6.75 (m, 3H), 4.55˜4.51 (s, 1H), 3.64˜3.49 (m, 2H), 2.99˜2.86 (m, 2H), 2.76˜2.66 (d, 2H), 2.50˜2.40 (m, 1H), 2.13˜2.05 (m, 2H), 1.84-1.77 (d, 2H), 1.23-1.26 (d, 6H), 1.19-1.18 (d, 3H). HPLC Column YMC ODS-AQ (0.46×5 cm×5 μm), RT=2.12 min, Mobile Phase 10% MeCN (0.1% TFA) in water to 80% MeCN ((0.1% TFA) in water. LCMS m/z 447.4 (M+1).

### Example 92

### Racemic (3R/S,5R,7S)(3R/S,5S,7R)˜1˜(3˜fluorophenyl)˜3˜hydroxy˜8˜(3˜isopropoxybenzyl)-7-methyl-1,8-diazaspiro[4.5]decan-2-one, hydrochloride salt (92)

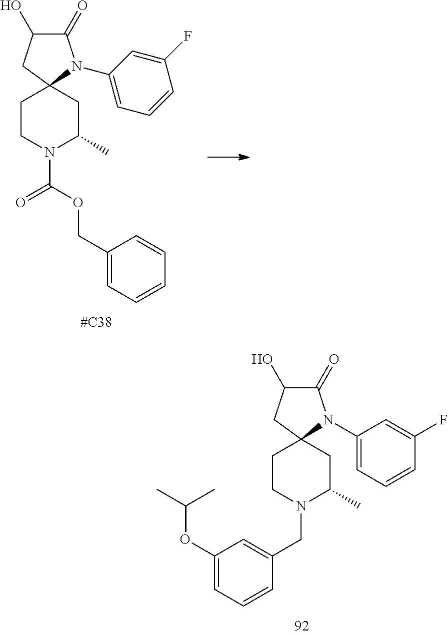

Compound #C38 was hydrogenated according to the procedure described in Preparation 4. The deprotected material [LCMS m/z 279.4 (M+1)] was then converted to the title product by reaction with 3˜isopropoxybenzaldehyde, using the general method described for preparation of 87 in Example 87, except that the gradient for chromatography was 0% to 5% methanol in dichloromethane. The free base was isolated as a colorless oil, estimated by ¹H NMR to be comprised of a roughly 3:2 mixture of diastereomers at the carbon bearing the hydroxy group. Yield: 9 mg, 0.02 mmol, 15%. ¹H NMR (400 MHz, CHCl₃): δ 1.12 (d, J=6.9 Hz) and 1.17 (d, J=6.7 Hz, 3H), 1.29 and 1.35 (2 d, J=6.0 Hz) and 1.30 (d, J=6.0 Hz, total 6H), 1.56-2.84 (m, 8H), 2.98-3.04 (m, 1H), 3.49 (AB quartet, J_{AB}=13.6 Hz, Δv_{AB}=75.6 Hz) and 3.50 (AB quartet, J_{AB}=13.6 Hz, Δv_{AB}=25.7 Hz, total 2H), 4.46˜4.64 (m, 2H), 6.73˜6.80 (m, 3H), 6.84-6.87 (m, 1H), 6.90-6.93 (m, 1H), 7.08-7.18 (m, 2H), 7.36-7.43 (m, 1H). LCMS m/z 427.1 (M+1). Conversion to the hydrochloride salt as in Example 87 provided 3.6 mg of the title product.

### Example @93

### (5R,7-(Cyclopropylmethyl)-8-(3-isopropoxybenzyl)-7-methyl-1,8-diazaspiro[4,5]dec-3-en-2-one (#93)

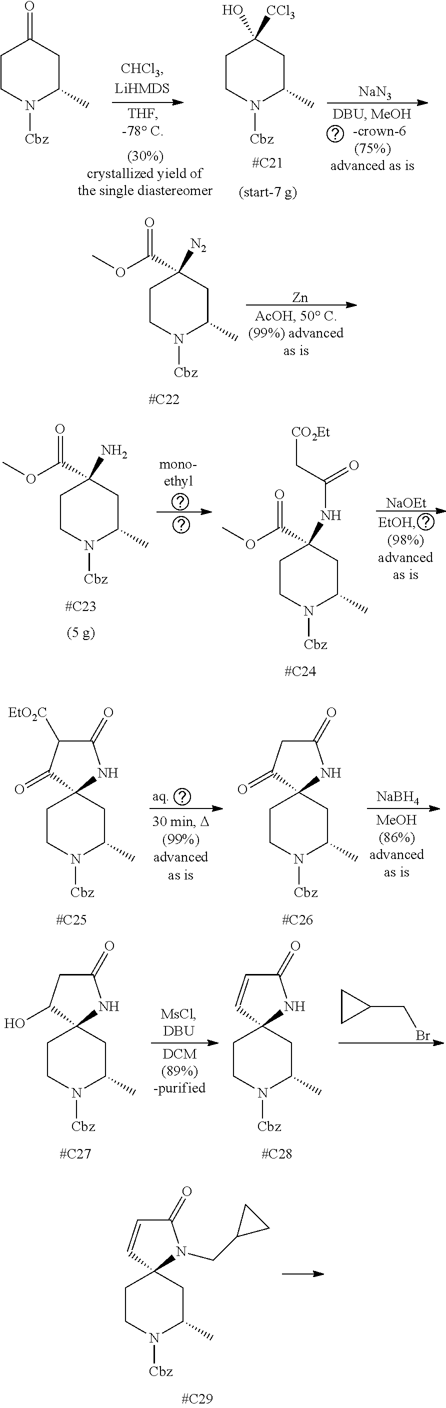

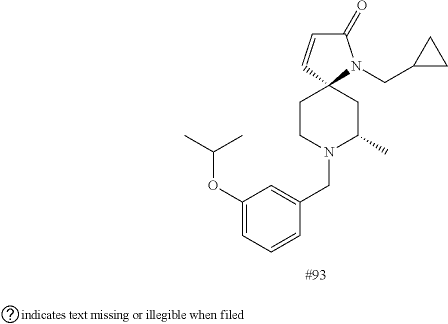

Step 1. Synthesis of benzyl (2S,4S)-4-hydroxy-2-methyl-4-(trichloromethyl)piperidine-1-carboxylate (#C21). Chloroform (4.06 mL, 50.7 mmol) was added to a mixture of benzyl (2S)-2-methyl-4-oxopiperidine-1-carboxylate (98.5%, 4.24 g, 16.9 mmol) and magnesium chloride (4.83 g, 50.7 mmol) in 1,2˜dimethoxyethane (45 mL), and the reaction mixture was cooled in a dry ice/acetone bath, Lithium bis(trimethylsilyl)amide (1 M in tetrahydrofuran, 25.4 mL, 25.4 mmol) was added drop-wise over 30 minutes, while keeping the internal temperature of the reaction below −72° C. The reaction was stirred at −72 to −77° C. for 4 hours, then allowed to warm to ˜15° C. by transferring the flask to a wet ice˜methanol bath. After one hour at ˜15° C., the reaction was slowly quenched with water (25 mL), then partitioned between water (75 mL) and ethyl acetate (150 mL). The aqueous phase was extracted with ethyl acetate (2×50 mL), and the combined organic extracts were washed with saturated aqueous sodium chloride solution (75 mL), dried over magnesium sulfate, filtered and concentrated in vacuo. The crude product was dissolved in diethyl ether (30 mL), which caused a white precipitate to form: this mixture was stirred for 18 hours. The solid was collected by filtration and rinsed with cold diethyl ether (10 mL) to provide #C21 as a white solid. The relative configuration of the methyl and hydroxy groups was determined by single-crystal X-ray crystallographic analysis of a sample prepared in an analogous manner; that sample was crystallized from acetonitrile˜water. Yield: 2.95 g, 8.05 mmol, 48%. ¹H NMR (400 MHz, DMSO-d₆, presumed to be a mixture of rotamers) δ 1.27 and 1.28 (2 d, J=6.9 Hz, 3H), 1.81-1.96 (m, 3H), 2.07-2.15 (m, 1H), 3.09-3.25 (m, 1H), 3.95-4.03 (m, 1H), 4.44˜4.53 (m, 1H), 5.04˜5.14 (m, 2H), 6.20 (s, 1H), 7.29˜7.40 (m, 5H).

Step 2. Synthesis of 1˜benzyl 4˜methyl (2S,4R)˜4˜azido˜2˜methylpiperidine˜1,4˜dicarboxylate (#C22). A suspension of benzyl (2S,4S)-4-hydroxy-2-methyl-4-(trichloromethyl)piperidine-1-carboxylate (#C21) (18.00 g, 49.09 mmol), 18-crown-6 ether (2.00 g, 7.57 mmol) and sodium azide (98%, 9.00 g, 136 mmol) in methanol (130 mL) was stirred at room temperature for 1 hour. 1,8˜Diazabicyclo[5.4.0]undec˜7˜ene (98%, 24.0 mL, 157 mmol) was then added over ten minutes. The reaction mixture was stirred at room temperature for 18 hours. Most of the methanol was removed in vacuo, and the residue was diluted with water (200 mL) and extracted with ethyl acetate (2×250 mL). The combined organic extracts were washed with water (150 mL), washed with saturated aqueous sodium chloride solution (150 mL) and dried over magnesium sulfate. After filtration and removal of solvent under reduced pressure, #C22 was obtained as a light yellow oil. Yield: 15.8 g, 47.5 mmol, 97%. APCI m/z 333.3 (M+1). ¹H NMR (400 MHz, CDCl₃) δ 1.09 (d, J=7.1 Hz, 3H), 1.60 (ddd, J=13.5, 12.5, 5.3 Hz, 1H), 1.94 (dd, J=13.6, 6.1 Hz, 1H), 2.23˜2.32 (m, 2H), 3.16 (ddd, J=14.3, 12.3, 3.2 Hz, 1H), 3.84 (s, 3H), 4.07 (br ddd, J=14, 5, 3 Hz, 1H), 4.45˜4.53 (m, 1H), 5.14 (s, 2H), 7.30˜7.40 (m, 5H).

Step 3. Synthesis of 1-benzyl 4-methyl (2S,4R)-4-amino-2-methylpiperidine-1,4-dicarboxylate (#C23). Zinc dust (99%, 4.76 g, 72 mmol) was added to a solution of 1˜benzyl 4˜methyl (2S,4R)˜4˜azido˜2˜methylpiperidine˜1,4˜dicarboxylate (#C22) (4.8 g, 14.4 mmol) in acetic acid (35 mL) and tetrahydrofuran (35 mL), and the reaction mixture was heated at 50° C. for 4 hours. After cooling to room temperature, the mixture was filtered through Celite, and the filtrate was concentrated in vacuo to remove most of the solvents. The residue was diluted with ethyl acetate, washed several times with saturated aqueous sodium bicarbonate solution, then washed with saturated aqueous sodium chloride solution and dried over magnesium sulfate. The mixture was filtered and concentrated under reduced pressure to provide #C23 as a light yellow oil, which was taken to the next step. Yield: 4.4 g, 14.4 mmol, quantitative. LCMS m/z 307.5 (M+1). ¹H NMR (400 MHz, CDCl₃) δ 1.05 (d, J=7.1 Hz, 3H), 1.44 (ddd, J=13.2, 12.8, 5.2 Hz, 1H), 1.73 (dd, J=13.6, 6.0 Hz, 1H), 2.15-2.26 (m, 4H), 3.16 (ddd, J=14.1, 12.7, 3.1 Hz, 1H), 3.75 (s, 3H), 4.05 (br ddd, J=14, 5, 3 Hz, 1H), 4.42˜4.50 (m, 1H), 5.14 (AB quartet, J_{AB}=12.5 Hz, Δv_{AB}=5.5 Hz, 2H), 7.29˜7.39 (m, 5H).

Step 4. Synthesis of 1˜benzyl 4˜methyl (2S,4R)˜4˜[(3˜ethoxy˜3˜oxopropanoyl)amino]˜2˜methylpiperidine˜1,4˜dicarboxylate (#C24). N˜[3˜(Dimethylamino)propyl]˜N′˜ethylcarbodiimide hydrochloride (EDCl, 98%, 3.58 g, 18.3 mmol) was added to a solution of 1-benzyl 4-methyl (2S,4R)-4-amino-2-methylpiperidine-1,4-dicarboxylate (#C23) (5.10 g, 16.6 mmol), 3-ethoxy-3-oxopropanoic acid (96%, 2.25 mL, 18.3 mmol) and triethylamine (99%, 4.69 mL, 33.3 mmol) in dichloromethane (50 mL), and the mixture was stirred at room temperature for 2 hours. An additional 0.1 equivalent of EDCl and 3-ethoxy-3-oxopropanoic acid were added, and stirring was continued for 1 hour. Solvents were removed in vacuo, and the residue was diluted with ethyl acetate, washed twice with 0.5 N aqueous hydrochloric acid, washed with saturated aqueous sodium bicarbonate solution, water, and saturated aqueous sodium chloride solution. After drying over magnesium sulfate, the mixture was filtered, and the filtrate concentrated under reduced pressure to provide #C24 as a viscous, light yellow oil, which was used without further purification. Yield: 7.3 g, >16.6 mmol, quantitative. LCMS m/z 421.5 (M+1). ¹H NMR (400 MHz, CDCl₃) δ 1.16 (d. J=6.8 Hz, 3H), 1.28 (t, J=7.2 Hz, 3H), 1.68 (ddd. J=14.0, 12.0, 6.2 Hz, 1H), 2.07 (br dd, half of ABX system, J=13.9, 6.0 Hz, 1H), 2.17 (dd, half of ABX system, J=13.8, 6.2 Hz, 1H), 2.52-2.58 (m, 1H), 3.24 (s, 2H), 3.34 (ddd, J=14.2, 12, 4.3 Hz. 1H). 3.75 (s, 3H), 4.02 (br ddd, J=14, 6, 2 Hz, 1H), 4.10˜4.24 (m, 2H), 4.27˜4.35 (m, 1H), 5.14 (AB quartet, J_{AB}=12.4 Hz, Δv_{AB}=6.0 Hz, 2H), 7.30-7.39 (m, 5H), 7.56 (br s, 1H).

Step 5 Synthesis of 8˜benzyl 3˜ethyl (5R,7S)˜7˜methyl˜2,4˜dioxo˜1,8˜diazaspiro[4.5]decane˜3,8˜dicarboxylate (#C25). Sodium ethoxide powder (95%, 1.41 g, 19.7 mmol) was added to a solution of 1˜benzyl 4˜methyl (2S,4R)˜4˜[(3˜ethoxy˜3˜oxopropanoyl)amino]˜2˜methylpiperidine˜1,4˜dicarboxylate (#C24) (6.90 g, 16.4 mmol) in methanol, and the mixture was stirred at room temperature for 20 minutes. The reaction was quenched with acetic acid (2 mL), and most of the ethanol was removed in vacuo. The residue was diluted with ethyl acetate, then washed with 0.2 N aqueous hydrochloric acid, water, and saturated aqueous sodium chloride solution. After drying over magnesium sulfate, the mixture was filtered and concentrated under reduced pressure to afford #C25 as a white foam whose NMR data indicated a mixture of diastereomers, which was taken to the next step without purification. Yield: 6.4 g, 16 mmol, 98%. LCMS m/z 389.5 (M+1). ¹H NMR (400 MHz, CDCl₃) δ 1.26-1.30 (m, 3H). 1.40 (t, J=7.1 Hz, 3H), 1.76-1.92 (m, 3H), 2.18 (ddd, J=14.2, 5.9, 2.5 Hz, 1H), 3.21-3.31 (m, 1H), 4.03-4.10 (m, 1H), 4.18-4.25 (m, 1H), 4.33-4.45 (m, 2H), 5.11˜5.22 (m, 2H), 6.31 (br s, 1H), 7.31˜7.41 (m, 5H).

Step 6. Synthesis of benzyl (5R,7S)-7-methyl-2,4-dioxo-1,8-diazaspiro[4.5]decane-8-carboxylate (#C26). 8-Benzyl 3-ethyl (5R,7S)-7-methyl-2,4-dioxo˜1,8˜diazaspiro[4.5]decane˜3,8˜dicarboxylate (#C25) (6.30 g, 16.2 mmol) was dissolved in dioxane (90 mL) and water (10 mL) and heated at reflux for 1 hour. After cooling to room temperature, the reaction was concentrated in vacuo. The residue was diluted with ethyl acetate, washed with saturated aqueous sodium chloride solution, dried over magnesium sulfate, filtered and concentrated under reduced pressure to afford #C26 as a light yellow foam. Yield: 5.13 g, 16.2 mmol, quantitative. LCMS m/z 317.5 (M+1). ¹H NMR (400 MHz, CDCl₃) δ 1.26 (d, J=6.6 Hz, 3H), 1.74-1.89 (m, 3H), 2.13 (ddd, J=14.1, 5.2, 2.3 Hz, 1H), 3.06 (AB quartet, J_{AB}=22.2 Hz, Δv_{AB}=38.5 Hz, 2H), 3.25 (ddd, J=14.2, 11.3, 5.1 Hz, 1H), 4.07 (br ddd, J=14, 7, 2 Hz, 1H), 4.24-4.33 (m, 1H), 5.16 (AB quartet, J_{AB}=12.3 Hz, Δv_{AB}=18.2 Hz, 2H), 6.70 (br s, 1H), 7.32-7.41 (m, 5H).

Step 7. Synthesis of benzyl (5R,7S)-4-hydroxy-7-methyl-2-oxo-1,8-diazaspiro[4,5]decane˜8˜carboxylate (#C27). Sodium borohydride (98%, 915 mg, 23.7 mmol) was added to a solution of benzyl (5R,7S)-7-methyl-2,4-dioxo-1,8-diazaspiro[4,5]decane-8-carboxylate (#C26) (5.00 g. 15.8 mmol) in methanol (100 mL) and the reaction was allowed to stir at room temperature for 18 hours. After the addition of more sodium borohydride (300 mg, 7.8 mmol), the reaction was stirred for one hour, then quenched with acetic acid (5.5 mL, 96 mmol) and concentrated in vacuo. The residue was diluted with ethyl acetate, washed with 0.2 N hydrochloric acid, saturated aqueous sodium bicarbonate solution, water, and saturated aqueous sodium chloride solution. The organic layer was dried over magnesium sulfate, filtered and concentrated under reduced pressure to provide #C27 as a viscous, colorless oil that was a mixture of diastereomers. Yield: 4.6 g, 14.4 mmol, 91% LCMS m/z 319.5 (M+1). ¹H NMR (400 MHz, CDCl₃) δ 1.24-1.28 (m, 3H), 1.56-1.81 (m, 3H), 2.06-2.11 and 2.24-2.36 (m, 2H), 2.77-2.85 (m, 1H), 3.04-3.20 (m, 1H), 4.00˜4.09 (m, 1H), 4.22˜4.30 (m, 1H), 4.31˜4.47 (m, 1H), 5.10˜5.18 (m, 2H), 6.21 and 6.35 (2 br s, 1H), 7.30˜7.40 (m, 5H).

Step 8. Synthesis of benzyl (5R,7S)-7-methyl-2-oxo-1,8-diazaspiro[4.5]dec-3-ene-8-carboxylate (#C28). Methanesulfonyl chloride (99.5%, 1.16 mL, 14.9 mmol) was added to a solution of benzyl (5R,7S)-4-hydroxy-7-methyl-2-oxo-1,8-diazaspiro[4.5]decane˜8˜carboxylate (#C27) (4.30 g, 13.5 mmol). After addition of triethylamine (99%, 2.47 mL, 17.5 mmol), the reaction mixture was stirred at room temperature for 1 hour. At this point, 1,8-diazabicyclo[5.4.0]undec-7-ene (98%, 2.68 mL, 17.6 mmol) was added, and stirring was continued for 3 hours. Additional 1,8˜diazabicyclo[5.4.0]undec˜7˜ene (1.48 mL, 9.53 mmol) was added, and the reaction was allowed to continue for 1 hour. Most of the solvent was removed in vacuo, and the residue was diluted with ethyl acetate, washed with 0.5 N aqueous hydrochloric acid, then with saturated aqueous sodium bicarbonate solution, water, and saturated aqueous sodium chloride solution. The aqueous layer was dried over magnesium sulfate, filtered and concentrated under reduced pressure, and the crude product was purified by chromatography on silica gel (Eluant: 10% methanol in ethyl acetate) to afford the product as a light yellow foam. Yield: 3.4 g, 11.3 mmol, 84%, LCMS m/z 301.4 (M+1). ¹H NMR (400 MHz, CDCl₃) δ 1.28 (d, J=7.0 Hz, 3H), 1.62 (ddd, J=13.7, 3.4, 1.6 Hz, 1H), 1.73-1.79 (m, 1H), 1.37 (ddd, J=13.5, 12.4, 5.2 Hz, 1H), 2.04 (dd, J=13.7, 6.6 Hz, 1H), 3.12 (ddd, J=14.3, 12.3, 3.6 Hz, 1H), 4.18 (br ddd, J=14, 5, 3 Hz, 1H), 4.52-4.60 (m, 1H), 5.16 (AB quartet, J_{AB}=12.4 Hz, Δv_{AB}=9.8 Hz, 2H), 6.07 (dd, J=5.9, 1.7 Hz, 1H), 6.30 (br s, 1H), 7.32-7.41 (m, 6H).

Step 9. Synthesis of benzyl (5R,7S)-1-(cyclopropylmethyl)-7-methyl-2-oxo-1,8-diazaspiro[4.5]dec-3-ene-8-carboxylate (#C29). A solution of benzyl (5R,7S)-7-methyl-2-oxo-1,8-diazaspiro[4.5]dec-3-ene-8-carboxylate (#C28) (45 mg, 0.15 mmol) in tetrahydrofuran (0.3 mL) was added to a suspension of sodium hydride (60% in mineral oil, 6.6 mg, 0.16 mmol) in tetrahydrofuran (0.3 mL). The reaction was stirred for 20 minutes after gas evolution ceased, then treated with a solution of (bromomethyl)cyclopropane (33.6 mg, 0.249 mmol) in tetrahydrofuran (0.3 mL). The reaction was heated to 60° C. for 20 minutes, at which time sodium iodide (<5 mg) and 15˜crown˜5 ether (1 drop from a Pasteur pipette, <5 mg) were added. The reaction mixture was maintained at 60° C. for an additional 6 hours, then at room temperature for 18 hours. Solvent was removed under a stream of nitrogen, and the residue was partitioned between water (1.5 mL) and ethyl acetate (3 mL). The aqueous layer was extracted with ethyl acetate (2 mL), and the combined organic layers were dried over magnesium sulfate, filtered and concentrated in vacuo. Purification was effect by chromatography on silica gel (Gradient: 0% to 10% methanol in dichloromethane) to provide the product as a thick gray oil. Yield: 52 mg, 0.147 mmol, 98%. LCMS m/z 355.2 (M+1). ¹H NMR (400 MHz, CDCl₃) δ 0.30˜4.34 (m, 2H), 0.49-0.54 (m, 2H), 0.99-1.09 (m, 1H), 1.30 (d, J=6.9 Hz, 3H), 1.3-1.39 (br m, 1H), 1.42˜1.54 (br m, 1H), 1.97˜2.09 (br m, 1H), 2.28 (dd, J=13.8, 6.5 Hz, 1H), 3.08˜3.24 (m, 3H), 4.16˜4.35 (br m, 1H), 4.61˜4.81 (br m, 1H), 5.13˜5.21 (m, 2H), 6.19 (d, J=6.2 Hz, 1H), 7.32-7.40 (m, 5H), 7.47 (d, J=6.2 Hz, 1H).

Step 10. Synthesis of (5R,7S)-1-(cyclopropylmethyl)-8-(3-isopropoxybenzyl)-7-methyl-1,8-diazaspiro[4.5]dec-3-en-2-one (#93). Benzyl (5R,7S)˜1˜(cyclopropyl methyl)˜7˜methyl˜2˜oxo˜1,8˜diazaspiro[4.5]dec˜3˜ene˜8˜carboxylate (#C29) (48 mg, 0.14 mmol) was dissolved in a freshly prepared solution of trimethylsilyl iodide (0.17 M in acetonitrile, 1.0 ml., 0.17 mmol), and the resulting solution was stirred at room temperature for 8 hours. Purification was carried out by loading the reaction mixture directly onto a mixed˜mode cation˜exchange (MCX) solid-phase extraction column. The column was flushed with dichloromethane (5 mL), and the product was then eluted using a 2 M solution of ammonia in methanol (5 mL). The eluant was concentrated in vacuo to afford the deprotected intermediate. LCMS m/z 221.1 (M+1). This material was mixed with acetonitrile (1 mL) and potassium carbonate (62.8 mg, 0.45 mmol). After addition of 1˜(bromomethyl)˜3˜isopropoxybenzene (which may be prepared from 3˜isopropoxybenzaldehyde using the general procedure reported by A. van Oeveren et al. J. Org. Chem. 1994, 59, 5999˜6007) (68.7 mg, 0.300 mmol), the mixture was stirred at room temperature for 1 hour, then loaded onto an MCX cartridge containing a small amount of Celite on top of the packing material, to assist in removing solids. The cartridge was flushed with dichloromethane (5 mL), and the filtered solids and Celite were manually removed from the cartridge. The product was eluted using a 2 M solution of ammonia in methanol (5 mL), and the filtrate was concentrated in vacuo. The residue was purified by preparative silica thin layer chromatography (Eluant: 5% acetonitrile in ethyl acetate); the product band was extracted with 2:1 ethyl acetate: methanol (15 mL) and filtered. After removed of solvent under reduced pressure, the residue was dissolved in ethyl acetate (3 mL), passed through a nylon filter (0.2 μm) and reconcentrated to provide the product as a gray/off-white semi-solid. Yield: 24.8 mg, 0.067 mmol, 48%. LCMS m/z 369.2 (M+1). ¹H NMR (400 MHz, CDCl₃) δ 0.35-0.40 (m, 2H), 0.49-0.54 (m, 2H), 1.05-1.13 (m, 1H), 1.15 (d, J=6.8 Hz, 3H), 1.33-1.46 (m, 2H), 1.35 (d, J=6.0 Hz, 6H), 2.11 (ddd, J=12, 12, 5 Hz, 1H), 2.34 (dd, J=13.1, 5.5 Hz, 1H), 2.61-2.73 (m, 2H), 3.19-3.35 (m, 3H), 3.62 (AB quartet, J_{AB}=13.7 Hz, Δv_{AB}=8.3 Hz, 2H). 4.58 (septet, J=6.0 Hz. 1H), 6.10 (d, J=6.2 Hz, 1H), 6.79 (br d, J=8 Hz, 1H), 6.90-6.94 (m, 2H), 7.23 (dd, J=8, 8 Hz, 1H), 7.37 (d, J=6.0 Hz, 1H).

### Example @94

### (5R,7S)-1-Isopropoxybenzyl)-7-methyl-1-propyl-1-diazaspiro[4.5]dec-3-en-2-one (#94)

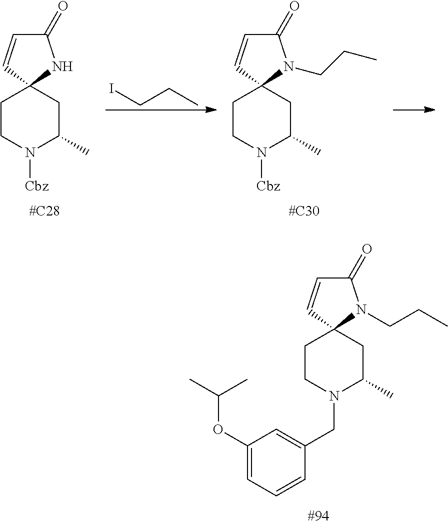

Step 1. Synthesis of benzyl (5R,7S)˜7˜methyl˜2˜oxo˜1˜propyl˜1,8˜diazaspiro[4.5]dec˜3˜ene˜8˜carboxylate (#C30). The title product was prepared according to the general procedure for the synthesis of benzyl (5R,7S)˜1˜(cyclopropylmethyl)˜7˜methyl˜2˜oxo˜1,8˜diazaspiro[4,5]dec˜3˜ene˜8˜carboxylate (#C29) in Example @93, except that 1˜iodopropane was used in place of (bromomethyl)cyclopropane, the reaction was heated at 60° C. for 22 hours, and sodium iodide and 15˜crown˜5 ether were not used. In this case, the crude product, obtained as a thick gray oil, was taken directly on to the next step. LCMS m/z 343.1 (M+1). ¹H NMR (400 MHz, CDCl₃), partial spectrum: δ 0.91 (t, J=7.4 Hz, 3H), 2.21 (dd, J=13.5, 6.6 Hz, 1H), 5.12-5.20 (m, 2H), 6.17 (d, J=6.2 Hz, 1H), 7.32-7.40 (m, 5H), 7.45 (d, J=6.2 Hz, 1H),

Step 2. Synthesis of (5R,7S)˜8˜(3˜isopropoxybenzyl)˜7˜methyl˜1˜propyl˜1,8-diazaspiro[4.5]dec˜3˜en˜2˜one (#94). The title compound was prepared according to the procedure described for the synthesis of #93 in Example @93, except that benzyl (5R,7S)-7-methyl-2-oxo-1-propyl-1,8-diazaspiro[4,5]dec-3-ene-8-carboxylate (#C30) was used instead of benzyl (5R,7S)-1-(cyclopropylmethyl)-7-methyl-2-oxo-1,8-diazaspiro[4.5]dec˜3˜ene˜8˜carboxylate (#C29). The product was obtained as a thick yellow oil. Yield: 14.3 mg, 0.040 mmol, 27% over 2 steps. LCMS m/z 357.6 (M+1). ¹H NMR (400 MHz, CDCl₃) δ 0.94 (t, J=7.4 Hz, 3H), 1.15 (d, J=6.8 Hz, 3H), 1.33˜1.38 (m, 1H), 1.36 (d, J=6.0 Hz, 6H), 1.41-1.47 (m, 1H), 1.60-1.70 (m, 2H), 2.00 (ddd, J=12.9, 10.7, 4.6 Hz, 1H), 2.23 (dd, J=13.2, 5.4 Hz, 1H), 2.59-2.73 (m, 2H), 3.22-3.34 (m, 3H), 3.62 (AB quartet, J_{AB}=13.6 Hz, Δv_{AB}=21.7 Hz, 2H), 4.58 (septet, Hz, 1H), 6.08 (d, J=6.0 Hz, 1H). 6.80 (br dd, J=8.3, 2.3 Hz, 1H), 6.91-6.94 (m, 2H), 7.23 (dd, J=7.8, 7.8 Hz, 1H), 7.31 (d, J=6.2 Hz, 1H).

### Example @95

### (5R,7S)-1-Cyclopropyl-8-(3-isopropoxybenzyl)-7-methyl-1,8-diazaspiro[4.5]dec-3-en-2˜one, trifluoroacetate salt (#95)

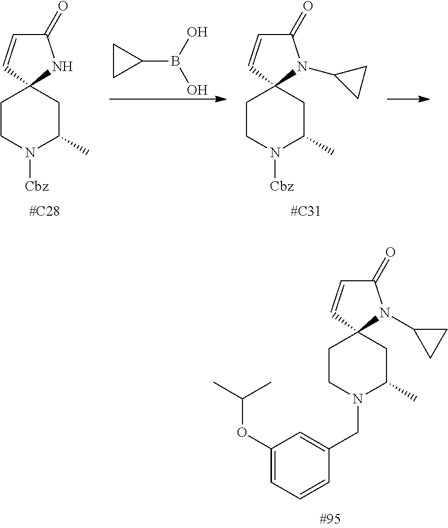

Step 1. Synthesis of benzyl (5R,7S)-1-cyclopropyl-7-methyl-2-oxo-1,8-diazaspiro[4.5]dec-3-ene-8-carboxylate (#C31). Benzyl (5R,7S)-7-methyl-2-oxo-1,8-diazaspiro[4.5]dec˜3˜ene˜8˜carboxylate (#C28) was converted into the title product by reaction with cyclopropylboronic acid, according to the method of S. Benard et al., J. Org. Chem. 2008, 73, 6441-6444. Purification was carried out by silica gel chromatography (Eluant: ethyl acetate) to provide the product as an oil. Yield: 18 mg, 0.053 mmol, 31%. LCMS m/z 341.3 (M+1). ¹H NMR (400 MHz, CDCl₃) δ 0.78-0.99 (m, 4H), 1.27˜1.3 (m, 1H), 1.30 (d, J=7.2 Hz, 3H), 1.36˜1.46 (br m, 1H), 2.17˜2.23 (m, 1H), 2.19-2.30 (br m, 1H), 2.55 (br dd, J=13, 7 Hz, 1H), 3.07-3.17 (br m, 1H), 4.17˜4.35 (br m, 1H), 4.66˜4.80 (br m, 1H), 5.13˜5.23 (m, 2H), 6.14 (d, J=6.2 Hz, 1H), 7.32-7.40 (m, 5H), 7.44 (d, J=6.2 Hz, 1H).

Step 2. Synthesis of (5R,7S)˜1˜cyclopropyl˜8˜(3˜isopropoxybenzyl)˜7˜methyl˜1,8˜diazaspiro[4.5]dec˜3˜en˜2˜one, trifluoroacetate salt (#95). The title compound was prepared according to the procedure described for the synthesis of #93 in Example @93, except that benzyl (5R,7S)-1-cyclopropyl-7-methyl-2-oxo-1,8-diazaspiro[4.5]dec˜3˜ene˜8˜carboxylate (#C31) was used instead of benzyl (5R,7S)˜1˜(cyclopropylmethyl)˜7˜methyl˜2˜oxo˜1,8˜diazaspiro[4.5]dec˜3˜ene˜8˜carboxylate (#C29), the removal of the protecting group was carried out over 18 hours rather than 8 hours, and strong cation exchange (SCX) solid-phase extraction columns were used rather than MCX columns. The final purification was carried out via reversed-phase HPLC (Column: C₁₈; Mobile phase A: 0.1% TFA in water (v/v); Mobile phase B: 0.1% TFA in acetonitrile (v/v); Gradient: 5% B to 100% B) to afford the title product as an oil, Yield: 7 mg, 0.015 mmol, 9% over 2 steps. LCMS m/z 355.2 (M+1). ¹H NMR (400 MHz, CD₃OD), partial spectrum: δ 0.81˜1.01 (br in, 4H), 1.33 (d, J=6.0 Hz, 6H), 1.62 (d, J=6.9 Hz, 3H), 1.65-1.74 (br m, 1H), 3.46-3.60 (br m, 2H), 4.67 (septet, J=6.0 Hz, 1H), 7.03˜4.23 (m, 4H), 7.41 (dd, J=7.9, 7.9 Hz, 1H).

### Example @96

### (5R,7S)˜1˜(3˜Fluorophenyl)˜8˜(3˜isopropoxybenzyl)˜3,7˜dimethyl˜1,8˜diazaspiro[4.5]decan˜2˜one, hydrochloride salt (#96)

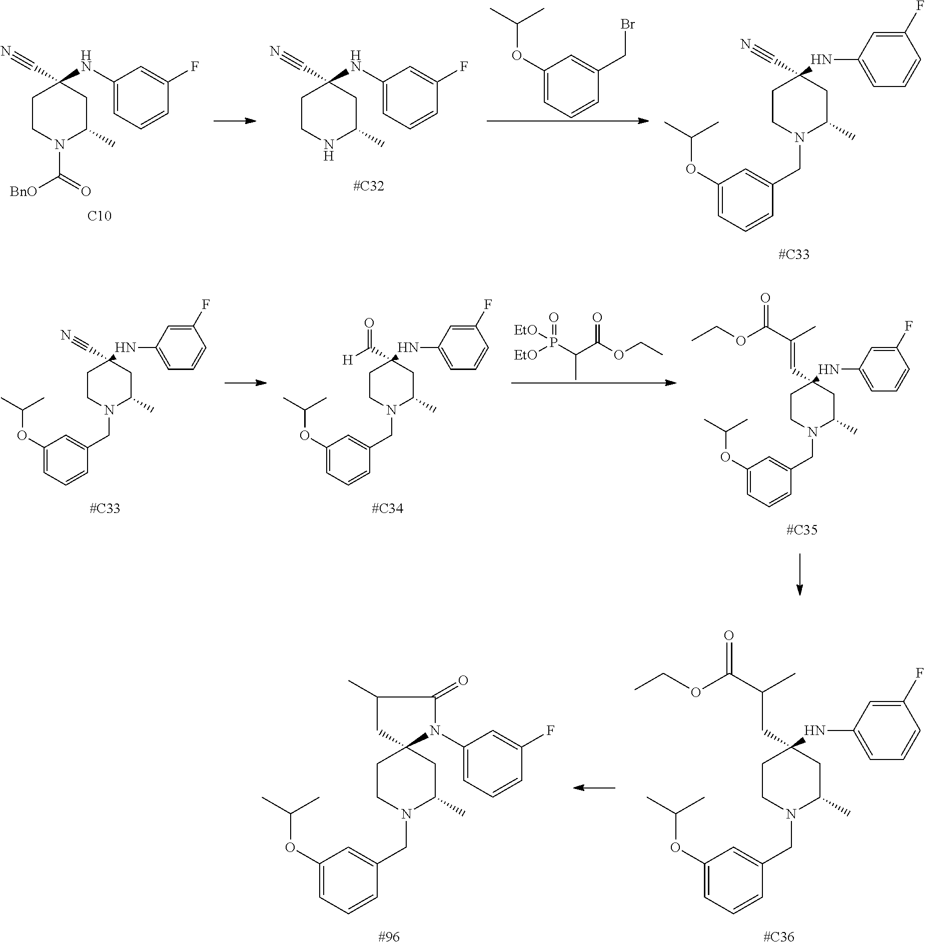

Step 1. Synthesis of (2S,4R)˜4˜[(3˜fluorophenyl)amino]˜2˜methylpiperidine˜4˜carbonitrile (#C32). Benzyl (2S,4R)-4-cyano-4-[(3-fluorophenyl)amino]-2-methylpiperidine-1-carboxylate (C10) (4.0 g, 11 mmol) was dissolved in methanol (100 mL) and treated with a suspension of palladium hydroxide on carbon (20% by weight, 540 mg, 0.77 mmol) in ethyl acetate (10 mL). The reaction mixture was shaken under 40 psi of hydrogen for 4 hours, filtered and concentrated in vacuo. The resulting oil was purified by chromatography on silica gel (Gradient: 50% to 100% ethyl acetate in heptane, followed by 10% methanol in ethyl acetate), to afford the product as a clear yellow oil. Yield: 1.85 g, 7.93 mmol, 72%, LCMS m/z 234.0 (M+1). ¹H NMR (400 MHz, CDCl₃) δ 1.10 (d, J=6.3 Hz, 3H), 1.43 (br s, 1H), 1.73 (dd, J=13.7, 11.5 Hz, 1H), 2.03 (ddd, J=13.8, 8.6, 8.6 Hz, 1H), 2.30-2.38 (m, 2H), 2.90-3.01 (m, 3H), 3.78 (br s, 1H), 6.56˜6.64 (m, 2H), 6.66 (ddd, J=8.1, 2.3, 0.8 Hz, 1H), 7.20 (ddd, J=8.2, 8.1, 6.7 Hz, 1H),

Step 2. Synthesis of (2S,4R)-4-[(3-fluorophenyl)amino]-1-(3-isopropoxybenzyl)-2-methylpiperidine-4-carbonitrile (#C33). (2S,4R)-4-[(3-Fluorophenyl)amino]˜2˜methylpiperidine˜4˜carbonitrile (#C32) (4.20 g, 18.0 mmol), 1˜(bromomethyl)˜3˜isopropoxybenzene (4.95 g, 21.6 mmol) and cesium carbonate (99%, 14.2 g, 43.1 mmol) were combined in acetonitrile (90 mL) and stirred at room temperature for 3 hours. The reaction mixture was then diluted with ethyl acetate, and washed with water, then with saturated aqueous sodium chloride solution, dried over magnesium sulfate, filtered and concentrated in vacuo. Purification was effected by chromatography on silica gel (Gradient 0% to 20% ethyl acetate in heptane), providing the product as a solid. Yield: 4.60 g, 12.1 mmol, 67%. The characterization data was obtained on a sample derived from a similar reaction. LCMS m/z 382.0 (M+1). ¹H NMR (400 MHz, CDCl₃) δ 1.22 (d, J=6.2 Hz, 3H), 1.35 (d, J=6.0 Hz, 6H), 2.02˜2.32 (m. 5H), 2.56˜2.71 (m, 2H), 3.06 (d, J=13.4 Hz, 1H), 3.80 (br s, 1H), 4.07 (d, J=13.4 Hz, 1H), 4.57 (septet, J=6.0 Hz, 1H), 6.55-6.66 (m, 3H), 6.77-6.88 (m, 3H), 7.16-7.24 (m, 2H).

Step 3. Synthesis of (2S,4R)-4-[(3-fluorophenyl)amino]-1-(3-isopropoxybenzyl)˜2˜methylpiperidine˜4˜carbaldehyde (#C34). Diisobutylaluminum hydride (98%, 1.5 M solution in toluene, 5.8 mL, 8.5 mmol) was added drop-wise to a solution of (2S,4R)-4-[(3-fluorophenyl)amino]-1-(3-isopropoxybenzyl)-2-methylpiperidine˜4˜carbonitrile (#C33) (2.20 g, 5.77 mmol) at ˜78° C. The reaction was stirred at ˜78° C. for 1 hour, then warmed to 0° C. for 1 hour, then warmed to room temperature for 1 hour. Aqueous ammonium chloride solution and 1 N hydrochloric acid were added until the reaction mixture was acidic (pH around 5). The aqueous layer was extracted three times with ethyl acetate, and the combined organic layers were dried, filtered and concentrated in vacuo. The residue was purified by chromatography on silica gel (Gradient: 0% to 100% ethyl acetate in heptane), providing the product as an oil. Yield: 730 mg, 1.90 mmol, 33%. LCMS m/z 385.0 (M+1). ¹H NMR (400 MHz, CDCl₃) δ 1.22 (d, J=6.0 Hz, 3H), 1.36 (d, J=6.0 Hz, 6H), 1.71 (dd, 11.7 Hz, 1H), 1.86-1.96 (m, 3H), 2.15 (br ddd, 11.3, 4.4 Hz, 1H), 2.48˜2.56 (m, 1H), 2.76 (br ddd, J=12, 3, 3 Hz, 1H), 3.07 (d, J=13.5 Hz, 1H), 4.12 (d, J=13.5 Hz, 1H), 4.17 (br s, 1H), 4.58 (septet, J=6.0 Hz, 1H), 6.25 (ddd, J=11.3, 2.3, 2.3 Hz, 1H), 6.30 (dd, J=8.0, 2.1 Hz, 1H), 6.44 (ddd, J=8.3, 8.3, 2.2 Hz, 1H), 6.79 (dd, J=8.1, 2.2 Hz, 1H), 6.86-6.91 (m, 2H), 7.08 (ddd, J=8.1, 8.1, 6.8 Hz, 1H), 7.21 (dd, J=7.9, 7.9 Hz, 1H), 9.63 (s, 1H).

Step 4 Synthesis of ethyl 3˜[(2S,4R)˜4˜[(3˜fluorophenyl)amino]˜1˜(3˜isopropoxybenzyl)˜2˜methylpiperidin˜4˜yl]˜2˜methylacrylate (#C35). Ethyl 2˜(diethoxyphosphoryl)propanoate (0.122 mL, 0.560 mmol) was added drop-wise to a mixture of sodium hydride (60% in oil, 20.6 mg, 0.515 mmol) in 1,2˜dimethoxyethane (0.9 mL) at 0° C. After being stirred at 0° C. for 30 min, the reaction was warmed to room temperature. (2S,4R)-4-[(3-Fluorophenyl)amino]-1-(3-isopropoxybenzyl)-2-methylpiperidine-4-carbaldehyde (#C34) (180 mg, 0.47 mmol) in minimal 1,2-dimethoxyethane was added drop-wise, and the reaction was stirred for an additional 3 hours. After addition of water, the mixture was extracted with ethyl acetate. The combined organic layers were dried over sodium sulfate, filtered and concentrated in vacuo. Purification via silica gel chromatography (Gradient: 10% to 40% ethyl acetate in heptane) afforded the title product as an oil. Yield: 85 mg, 0.18 mmol, 38%. LCMS m/z 469.1 (M+1). NMR (400 MHz, CDCl₃), characteristic signals: δ 1.18 (d. J=6.0 Hz, 3H), 1.31 (t, J=7.1 Hz, 3H), 1.34 (d, J=6.2 Hz, 6H), 1.94 (d, J=1.5 Hz, 3H), 3.04 (d, J=13.5 Hz, 1H), 4.10 (d, J=13.5 Hz, 1H), 4.21 (q, J=7.1 Hz, 2H), 7.04 (ddd, J=8.1, 8.1, 6.8 Hz, 1H).

Step 5. Synthesis of ethyl 3-[(2S,4R)-4-[(3-fluorophenyl)amino]-1-(3-isopropoxybenzyl)˜2˜methylpiperidin˜4˜yl]˜2˜methylpropanoate (#C36). Ethyl 3-[(2S,4R)˜4˜[(3˜fluorophenyl)amino]˜1˜(3˜isopropoxybenzyl)˜2˜methylpiperidin˜4˜yl]˜2˜methylacrylate (#C35) (85 mg, 0.18 mmol) and palladium on carbon (10%, 19.2 mg, 0.018 mmol) were combined in methanol (1.8 mL) and shaken under 50 psi of hydrogen for 18 hours. The reaction was filtered and concentrated in vacuo to provide the product as an oil, which was used without further purification. Yield: 75 mg, 0.16 mmol, 88%. LCMS m/z 471.4 (M+1).

Step 6. Synthesis of (5R,7S)-1-(3-fluorophenyl)-8-(3-isopropoxybenzyl)-3,7-dimethyl˜1,8˜diazaspiro[4.5]decan˜2˜one, hydrochloride salt (#96). Ethyl 3˜[(2S,4R)˜4-[(3-fluorophenyl)amino]-1-(3-isopropoxybenzyl)-2-methylpiperidin-4-yl]-2-methylpropanoate (#C36) (75 mg, 0.16 mmol) was added to a mixture of sodium hydride (9.5 mg. 0.24 mmol) and tetrahydrofuran (0.8 mL) at 0° C. The reaction was stirred under ice cooling for 1 hour, then heated at reflux for 18 hours. Removal of solvent under reduced pressure was followed by chromatography on silica gel (Gradient: 0% to 100% ethyl acetate in heptane) to provide the free base of the product as a roughly 2:1 mixture of diastereomers, as judged from the ¹H NMR spectrum. Yield: 14 mg, 0.033 mmol, 21%. LCMS m/z 425.0 (M+1). ¹H NMR (400 MHz, CDCl₃) δ 1.09 and 1.14 (2 d, J=6.9 and 6.8 Hz, 3H), 1.28˜1.32 (m, 9H), 1.46˜1.51 and 1.60˜1.77 (2 m, 3H), 1.82˜1.91 (m, 1H), 1.98˜2.08 (m, 1H), 2.19˜2.25 and 2.47˜2.76 (2 m, 4H), 2.89˜3.03 (m, 1H), 3.47 (AB quartet, J_{AB}=13.6 Hz, Δv_{AB}=22.8 Hz) and 3.44 (AB quartet, J_{AB}=13.6 Hz, Δv_{AB}=99.9 Hz, total of 2H), 4.45-4.55 (2 septets, J=6.0 Hz, 1H), 6.72˜6.81 (m, 3H), 6.84 (ddd, J=9.5, 2.2, 2.2 Hz, 1H), 6.90 (ddd, J=7.9, 1.8, 0.9 Hz, 1H). 7.06˜7.18 (m, 2H). 7.35˜7.42 (m, 1H).

This material was converted to 15 mg of the corresponding hydrochloride salt, isolated as a solid.

### Example @97

### (5R,7S)-1-(3-Fluorophenyl)-8-(4-hydroxy-3-isopropoxybenzyl)-7-methyl-3-phenyl-1,8-diazaspiro[4.5]dec-3-en-2-one, hydrochloride salt (#97)

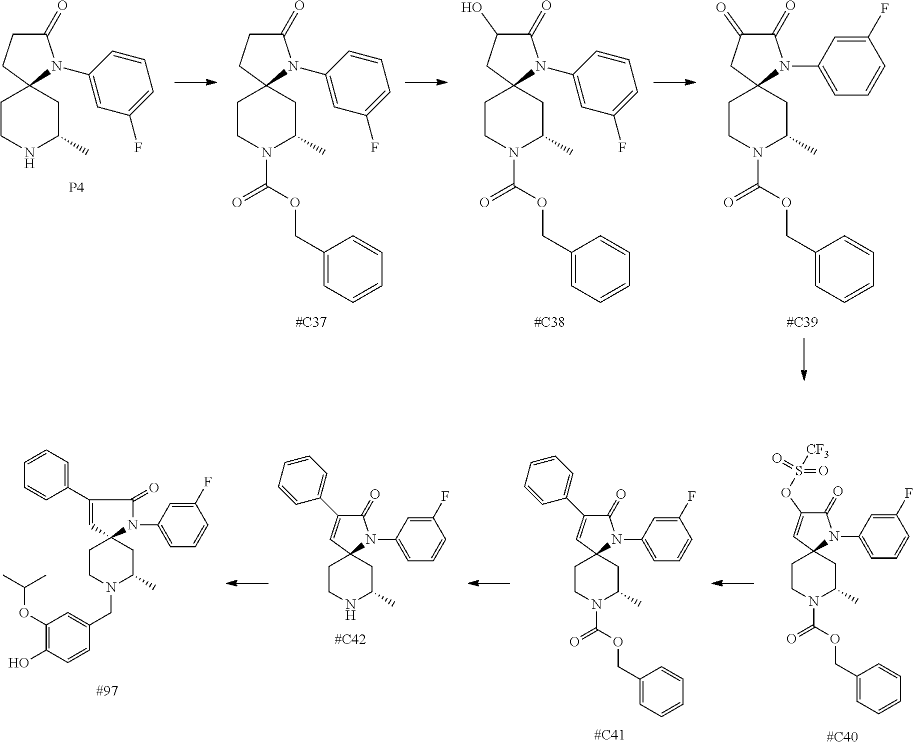

Step 1. Synthesis of benzyl (5R,7S)-1-(3-fluorophenyl)-7-methyl-2-oxo-1,8-diazaspiro[4.5]decane-8-carboxylate (#C37). (5R,7S)-1-(3-Fluorophenyl)-7-methyl-1,8-diazaspiro[4.5]decan-2-one (P4) (532 mg, 2.03 mmol) was dissolved in tetrahydrofuran (10 mL) and water (5 mL) and chilled in an ice bath. Sodium hydroxide (487 mg, 12.2 mmol) in water (1 mL) was added, followed by benzyl chloroformate (039 mL, 2.6 mmol), and the ice bath cooling the reaction mixture was allowed to warm to room temperature over 18 hours. The reaction was then poured into dilute aqueous sodium bicarbonate solution and extracted three times with dichloromethane. The combined organic layers were dried over sodium sulfate, filtered and concentrated in vacuo to provide a residue, which was subjected to silica gel chromatography (Gradient: 0% to 4% methanol in dichloromethane). The product was isolated as a white foam. Yield: 545 mg, 1.37 mmol, 67%.

Step 2. Synthesis of benzyl (5R,7S)-1-(3-fluorophenyl)-3-hydroxy-7-methyl-2-oxo-1,8-diazaspiro[4,5]decane-8-carboxylate (#C38). Lithium bis(trimethylsilyl)amide (1 M in tetrahydrofuran, 1.5 mL, 1.5 mmol) was added to a solution of benzyl (5R,7S)˜1˜(3˜fluorophenyl)˜7˜methyl˜2˜oxo˜1,8˜diazaspiro[4.5]decane˜8˜carboxylate (#C37) (500 mg, 1.26 mmol) in tetrahydrofuran (6.3 mL) at ˜60° C., and the reaction mixture was maintained at this temperature for 1 hour. A solution of 3-phenyl-2-(phenylsulfonyl)oxaziridine (see L. C. Vishwakarma et al., Organic Syntheses 1988, 66, 203-10) (494 mg, 1.89 mmol) in tetrahydrofuran was added drop-wise, and the reaction was warmed to room temperature and stirred for 18 hours. The mixture was poured into saturated aqueous ammonium chloride solution (3 mL) and extracted with dichloromethane (3×3 mL); the combined organic layers were dried over sodium sulfate, filtered and concentrated in vacuo. Purification via silica gel chromatography (Gradient: 0% to 5% methanol in dichloromethane) provided the product as a white solid, presumed to be a mixture of rotamers and diastereomers from its ¹H NMR spectrum, Yield: 201 mg, 0.487 mmol, 39%. APCI m/z 413.2 (M+1). ¹H NMR (400 MHz, CDCl₃) δ 1.24˜1.31 (m, 3H), 1.53˜1.94 (br m, 4H), 2.23 (dd, J=13.5, 5.3 Hz) and 2.05˜2.12 (m, total of 1H), 2.64 and 2.86 (2 dd, J=13.4, 8.1 Hz and J=12.8, 8.5 Hz, 1H), 3.05˜3.15 (br m, 1H), 4.05˜4.62 (m, 4H), 5.01˜5.12 (br s, 2H), 6.79-6.83 (m, 1H), 6.85-6.88 (m, 1H), 7.10-7.16 (m, 1H), 7.28-7.44 (m, 6H).

Step 3. Synthesis of benzyl (5R,7S)-1-(3-fluorophenyl)-7-methyl-2,3-dioxo-1,8-diazaspiro[4.5]decane-8-carboxylate (#C39). Manganese(IV) oxide (85%, 124 mg, 1.21 mmol) was added to a solution of benzyl (5R,7S)˜1˜(3˜fluorophenyl)˜3˜hydroxy˜7˜methyl˜2˜oxo˜1,8˜diazaspiro[4.5]decane˜8˜carboxylate (#C38) (50 mg, 0.12 mmol) in dichloromethane (0.61 mL), and the reaction was stirred at room temperature until no starting material was observed by thin layer chromatography on silica gel (Eluant: 5% methanol in chloroform). The reaction mixture was filtered through a <1 μm filter, and solvent was removed in vacuo. Chromatography on silica gel (Gradient; 0% to 5% methanol in dichloromethane) provided the product as an oil, assumed to be a mixture of rotamers from its ¹H NMR spectrum. Yield: 30 mg, 0.073 mmol, 61%.

LCMS m/z 411.0 (M+1). ¹H NMR (400 MHz, CDCl₃) δ 1.23-1.31 (m, 3H), 1.61˜1.78 (br m, 2H), 1.84˜2.13 (br m, 2H), 2.96 (AB quartet, J_{AB}=19.1 Hz, Δv_{AB}=34.9 Hz, 2H), 3.0-3.13 (br m, 1H), 4.12-4.31 (br m, 1H), 4.54-4.71 (br m, 1H), 5.00-5.14 (br m, 2H), 6.80˜6.94 (m, 2H), 7.11˜7.23 (m, 1H), 7.28˜7.39 (m, 5H), 7.44˜7.51 (m, 1H).

Step 4. Synthesis of benzyl (5R,7S)-1-(3-fluorophenyl)-7-methyl-2-oxo-3-{[(trifluoromethyl)sulfonyl]oxy}˜1,8˜diazaspiro[4.5]dec˜3˜ene˜8˜carboxylate (#C40). Lithium bis(trimethylsilyl)amide (1 M in tetrahydrofuran, 0.067 mL, 0.067 mmol) was added drop-wise to a solution of benzyl (5R,7S)-1-(3-fluorophenyl)-7-methyl-2,3-dioxo˜1,8˜diazaspiro[4.5]decane˜8˜carboxylate (#C39) (25 mg, 0.061 mmol) in tetrahydrofuran (0.61 mL) at −78° C. After 30 minutes, N-(5-chloropyridin-2-yl)-1,1,1-trifluoro˜N˜[(trifluoromethyl)sulfonyl]methanesulfonamide (28.7 mg, 0.0731 mmol) in tetrahydrofuran (1 mL) was added drop-wise and stirring was continued at −78° C. for 2 hours. Sodium sulfate decahydrate (100 mg, 0.31 mmol) was added, and the reaction was allowed to warm to room temperature, at which point it was filtered and concentrated in vacuo. Purification via silica gel chromatography (Gradient: 0% to 40% ethyl acetate in heptane) provided the product as a solid. Yield: 30 mg, 0.055 mmol, 90%. LCMS m/z 542.9 (M+1). ¹H NMR (400 MHz, CDCl₃) δ 1.31 (d, J=7.1 Hz, 3H), 1.55 (br d, J=13 Hz, 1H), 1.71˜1.78 (br m, 1H), 1.96˜2.06 (br m, 1H), 2.19 (br dd, J=13, 7 Hz, 1H), 3.06-3.17 (br m, 1H), 4.21-4.36 (br m, 1H), 4.60-4.74 (br m, 1H), 5.09 (br s, 2H), 6.86 (ddd, J=9.1, 2.2, 2.2 Hz, 1H), 6.91 (ddd, J=7.9, 1.9, 0.8 Hz, 1H), 7.18 (dddd, J=8.3, 8.3, 2.5, 0.8 Hz, 1H), 7.29-7.39 (m, 5H), 7.42 (s, 1H), 7.45 (ddd, J=8.2, 8.2, 6.2 Hz, 1H).

Step 5. Synthesis of benzyl (5R,7S)-1-(3-fluorophenyl)-7-methyl-2-oxo-3-phenyl˜1,8˜diazaspiro[4.5]dec˜3˜ene˜8˜carboxylate (#C41). Phenylboronic acid (8.0 mg, 0.066 mmol), anhydrous potassium phosphate (35.0 mg, 0.165 mmol) and then [1,1′˜bis(diphenylphosphino)ferrocene]dichloropalladium(II) (4.4 mg. 0.0060 mmol) were added to a solution of benzyl (5R,7S)-1-(3-fluorophenyl)-7-methyl-2-oxo-3-{[(trifluoromethyl)sulfonyl]oxy}-1,8-diazaspiro[4.5]dec-3-ene-8-carboxylate (#C40) (30 mg, 0.055 mmol) in tetrahydrofuran (0.55 mL). The resulting solution was heated at reflux for 1 hour, then cooled to room temperature, diluted with ethyl acetate (5 and filtered through a <1 μm filter. The filtrate was concentrated in vacuo, then purified by silica gel chromatography (Gradient: 0% to 50% ethyl acetate in heptane) to provide the product as a colorless oil. Yield: 20 mg, 0.042 mmol, 76% APCI m/z 471.1 (M+1). ¹H NMR (400 MHz, CDCl₃) δ 1.39 (d, J=7.2 Hz, 3H), 1.54-1.6 (m, 1H, assumed: partially obscured by water peak), 171˜1.79 (br m, 1H), 1.95˜2.05 (br m, 1H), 2.18 (br dd, J=13, 7 Hz, 1H), 3.19-3.28 (br m, 1H), 4.20-4.37 (br m, 1H), 4.62-4.74 (br m, 1H), 5.11 (br 5, 2H), 6.91 (ddd, J=9.3, 2.2, 2.2 Hz, 1H), 6.95˜6.98 (m, 1H), 7.16 (br ddd, J=8.3, 8.3, 2.5 Hz, 1H), 7.31-7.48 (m, 9H), 7.73 (s, 1H), 7.91-7.95 (m, 2H).

Step 6. Synthesis of (5R,7S)-1-(3-fluorophenyl)-7-methyl-3-phenyl-1,8-diazaspiro[4,5]dec-3-en-2-one (#C42). The title compound was prepared according to the general procedure for the synthesis of P1 in Preparation 1, except that benzyl (5R,7S)˜1˜(3˜fluorophenyl)˜7˜methyl˜2˜oxo˜3˜phenyl˜1,8˜diazaspiro[4.5]dec˜3˜ene˜8˜carboxylate (#C41) was used instead of racemic benzyl (5R,7S)(5S,7R)˜1˜(3˜fluorophenyl)˜7˜methyl˜2˜oxo˜1,8˜diazaspiro[4.5]dec˜3˜ene˜8˜carboxylate (C9). The product was obtained as an oil. Yield: 6.8 mg, 0.020 mmol, 47%. LCMS m/z 337.1 (M+1). ¹H NMR (400 MHz, CDCl₃) δ 1.07 (d, J=6.4 Hz, 3H), 1.72 (dd, J=14.2, 10.1 Hz, 1H), 1.90˜1.98 (m, 2H), 2.06 (ddd, J=14.2, 11.1, 5.0 Hz, 1H), 2.69 (ddd, J=12.7, 11.0, 3.2 Hz, 1H), 2.74-2.84 (m, 1H), 2.95 (ddd, J=12.6, 4.5, 4.5 Hz, 1H), 6.99 (ddd, J=9.3, 2.1, 2.1 Hz, 1H), 7.06 (ddd, J=7.9, 1.8, 0.9 Hz, 1H), 7.15 (dddd, J=8.4, 8.4, 2.5, 1.0, 1H), 7.17 (s, 1H), 7.34-7.47 (m, 4H), 7.91-7.94 (m, 2H).

Step 7. Synthesis of (5R,7S)˜1˜(3˜fluorophenyl)˜8˜(4˜hydroxy˜3˜isopropoxybenzyl)˜7˜methyl˜3˜phenyl˜1,8˜diazaspiro[4.5]dec˜3˜en˜2˜one, hydrochloride salt (#97). The title product was prepared from (5R,7S)-1-(3-fluorophenyl)-7-methyl-3-phenyl-1,8-diazaspiro[4.5]dec-3-en-2-one (#C42) according to the general procedure for the synthesis of (5R,7S)-1-(3-fluorophenyl)-8-(4-hydroxy˜3˜isopropoxybenzyl)˜7˜methyl˜1,8˜diazaspiro[4.5]dec˜3˜en˜2˜one hydrochloride (87) in Example 87, except that the purification was carried out by multiple silica gel chromatographies: 0% to 5% methanol in dichloromethane gradient, followed by 1% to 100% ethyl acetate in heptane gradient, and finally diethyl ether eluant, providing the neutral form of the product as a solid. Yield: 3.0 mg, 0.0056 mmol, 28%. LCMS m/z 500.2 (M+1). ¹H NMR (400 MHz, CDCl₃) δ 1.20 (d, J=6.7 Hz, 3H), 1.34 (br d, J=6 Hz, 6H), 1.64˜1.69 (m. 1H), 1.76˜1.82 (m, 1H), 2.00-2.08 (m, 1H), 2.18 (dd, J=13, 5 Hz, 1H), 2.41-2.47 (m, 1H), 2.68-2.74 (m, 1H), 2.99-3.04 (m, 1H), 3.48 (AB quartet, J_{AB}=13 Hz, Δv_{AB}=79 Hz, 2H), 4.54 (septet, Hz, 1H), 5.62 (s, 1H), 6.70 (dd, J=8, 2 Hz, 1H), 6.79 (d, J=2 Hz, 1H), 6.83 (d, J=8.0 Hz, 1H). 6.93˜6.96 (m, 1H), 6.99˜7.02 (m, 1H), 7.12˜7.17 (m, 1H), 7.34˜7.46 (m, 4H), 7.53 (s, 1H), 7.91-7.94 (m, 2H). The hydrochloride salt was prepared using 1 M hydrogen chloride in diethyl ether, providing #97 as a solid, 3 mg.

### Example @98

### (5R,7S)-1-(3-Fluorophenyl)-8-(4-hydroxy-3-isopropoxybenzyl)-3,7-dimethyl-1,8-diazaspiro[4.5]dec-3-en-2-one, hydrochloride salt (#98)

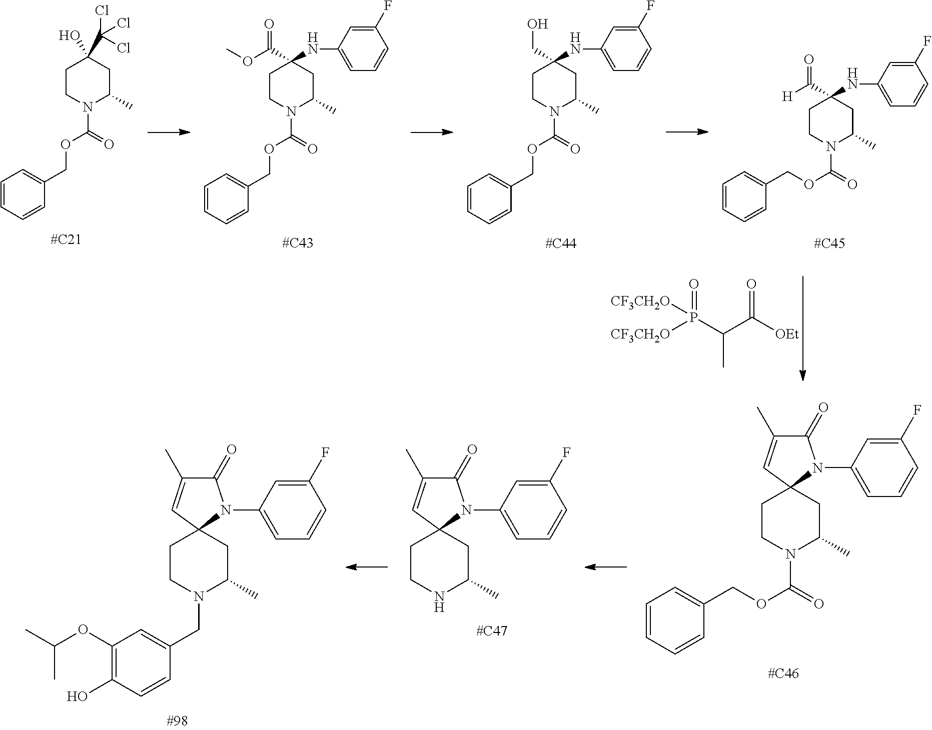

Step 1. Synthesis of 1˜benzyl 4˜methyl (2S,4R)˜4˜[(3˜fluorophenyl)amino]˜2˜methylpiperidine˜1,4˜dicarboxylate (#C43). Benzyl (2S,4S)˜4˜hydroxy˜2˜methyl˜4˜(trichloromethyl)piperidine-1-carboxylate (#C21) (4.80 g, 13.1 mmol), 3-fluoroaniline (98%, 2.91 mL. 26.2 mmol) and diazabicyclo[5.4.0]undec-7-ene (98%, 5.99 mL, 39.3 mmol) were dissolved in methanol (131 mL) and heated at reflux overnight. The reaction mixture was diluted with water and extracted with ethyl acetate. The combined organic layers were washed with saturated aqueous sodium chloride solution, dried over magnesium sulfate, filtered and concentrated in vacuo. The residue was purified by chromatography on silica gel (Eluant: 25% ethyl acetate in heptane) to afford a viscous, colorless oil (3.8 g), which was taken directly to the next step. LCMS m/z 401.47 (M+1).

Step 2. Synthesis of benzyl (2S,4R)-4-[(3-fluorophenyl)amino]-4-(hydroxymethyl)-2-methylpiperidine-1-carboxylate (#C44). 1-Benzyl 4-methyl (2S,4R)-4-[(3-fluorophenyl)amino]-2-methylpiperidine-1,4-dicarboxylate (#C43) from the previous step was dissolved in tetrahydrofuran (63.3 mL) and treated with a solution of lithium borohydride in tetrahydrofuran (2 M, 19.0 mL, 38.0 mmol). The resulting mixture was heated at reflux for 18 hours. After cooling to room temperature, the reaction mixture was quenched with saturated aqueous ammonium chloride solution, diluted with water, and extracted with ethyl acetate. The combined organic layers were washed with saturated aqueous sodium chloride solution, dried over magnesium sulfate, filtered and concentrated under reduced pressure. The residue was purified by silica gel chromatography (Eluant: 50% ethyl acetate in heptane) to provide the product as a viscous, colorless oil. Yield: 1.10 g, 2.95 mmol, 22% over two steps. ¹H NMR (400 MHz, CDCl₃) δ 1.21 (d, J=6.6 Hz, 3H), 1.35 (dd, J=14.4, 7.9 Hz, 1H), 1.78˜1.88 (m, 2H), 2.06 (dd, J=14.3, 6.5 Hz, 1H), 2.20 (br J=6, 6 Hz, 1H), 3.06-3.14 (m, 1H), 3.53 (br s, 1H), 3.68 (dd, half of ABX system, J=11.3, 4.9 Hz, 1H), 3.75 (dd, half of ABX system, J=11.3, 6.1 Hz, 1H), 3.96-4.03 (m, 1H), 4.15-4.24 (m, 1H), 5.13 (s, 2H), 6.39-6.44 (m, 2H), 6.52 (dddd, 6.3, 2.3, 0.9 Hz, 1H), 7.08 (ddd, J=8.3, 8.3, 6.8 Hz, 1H), 7.29˜7.38 (m, 5H),

Step 3. Synthesis of benzyl (2S,4R)˜4˜[(3˜fluorophenyl)amino]˜4˜formyl˜2˜methylpiperidine-1-carboxylate (#C45). Oxalyl chloride (99%, 0.39 mL, 4.4 mmol) was added drop˜wise to a ˜78 solution of dimethyl sulfoxide (0.63 mL, 8.9 mmol) in dichloromethane (5 mL). After 20 minutes, a solution of benzyl (2S,4R)-4-[(3-fluorophenyl)amino]˜4˜(hydroxymethyl)˜2˜methylpiperidine˜1˜carboxylate (#C44) (1.10 g, 2.95 mmol) in dichloromethane (5 mL) was slowly added. After an additional 20 minutes, triethylamine (99%, 1.66 mL, 11.8 mmol) was added, and the reaction mixture was allowed to warm to room temperature and stir for 18 hours. The reaction was then diluted with water and extracted with ethyl acetate. The organic layer was washed with saturated aqueous sodium chloride solution, dried over magnesium sulfate, filtered and concentrated under reduced pressure to provide the product as an oil. Yield: 600 mg, 1.62 mmol, 55%. ¹H NMR (400 MHz, CDCl₃) δ 1.18 (d, J=6.7 Hz, 3H), 1.71 (ddd, J=13.7, 12.2, 6.0 Hz, 1H), 1.91 (dd, half of ABX system, J=14.2, 6.4 Hz, 1H), 1.98 (dd, half of ABX system, J=14.2, 6.3 Hz, 1H), 2.44 (ddd, J=14.0, 3, 3 Hz, 1H), 3.14 (ddd. J=14.2, 12.0, 4.2 Hz, 1H), 4.03˜4.09 (m, 2H), 4.32˜4.41 (m, 1H), 5.14 (s, 2H), 6.21 (ddd. J=11.2, 2.3, 2.3 Hz, 1H), 6.25 (br dd, J=8, 2 Hz, 1H), 6.46 (br ddd, J=8, 8, 2 Hz, 1H), 7.05 (ddd, J=8.1, 8.1, 6.7 Hz, 1H), 7.31-7.39 (m, 5H), 1H).

Step 4. Synthesis of benzyl (5R,7S)-1-(3-fluorophenyl)-3,7-dimethyl-2-oxo-1,8-diazaspiro[4.5]dec-3-ene-8-carboxylate (#C46). Ethyl 2-[bis(2,2,2-trifluoroethoxy)phosphoryl]propanoate (353 mg, 1.02 mmol) was added drop-wise to an ice-cooled mixture of sodium hydride (60% in oil, 40.8 mg, 1.02 mmol) and 1,2˜dimethoxyethane (1.46 mL). The mixture was stirred at 0° C. for 30 minutes and then warmed to room temperature. A solution of benzyl (2S,4R)˜4˜[(3˜fluorophenyl)amino]-4-formyl-2-methylpiperidine-1-carboxylate (#C45) (270 mg, 0.729 mmol) in minimal 1,2˜dimethoxyethane was added drop˜wise, and the reaction was stirred for 18 hours. Water was then added, and the mixture was extracted with ethyl acetate. The combined organic layers were dried over sodium sulfate, filtered and concentrated in vacuo. Purification of the residue via chromatography on silica gel (Gradient: 10% to 40% ethyl acetate in heptane) provided the product as a solid. Yield: 170 mg, 0.416 mmol, 57%. LCMS m/z 409.5 (M+1). ¹H NMR (400 MHz, CDCl₃) δ 1.32 (d, J=7.0 Hz, 3H), 1.41˜1.47 (m, 1H), 1.57˜1.65 (m, 1H), 1.82˜1.95 (m, 1H), 1.99 (d, J=16 Hz, 3H), 2.04˜2.10 (m, 1H), 3.08˜3.17 (m, 1H), 4.13˜4.26 (m, 1H), 4.56-4.69 (m, 1H), 5.05-5.14 (m, 2H), 6.84 (ddd, J=9.4, 2.2, 2.2 Hz, 1H), 6.89 (br d, J=8 Hz, 1H), 7.11 (br ddd, J=8.4, 8.4, 2.5 Hz, 1H), 7.23-7.24 (m, 1H), 7.30-7.37 (m, 5H), 7.41 (ddd, J=8.2, 8.2, 6.4 Hz, 1H).

Step 5. Synthesis of (5R,7S)˜1˜(3˜fluorophenyl)˜3,7˜dimethyl˜1,8˜diazaspiro[4.5]dec˜3˜en˜2˜one (#C47). The title compound was prepared according to the general procedure for the synthesis of P1 in Preparation 1, except that benzyl (5R,7S)˜1˜(3˜fluorophenyl)˜3,7˜dimethyl˜2˜oxo˜1,8˜diazaspiro[4.5]dec˜3˜ene˜8˜carboxylate (#C46) was used instead of racemic benzyl (5R,7S)(5S,7R)˜1˜(3˜fluorophenyl)˜7˜methyl˜2˜oxo˜1,8˜diazaspiro[4.5]dec˜3˜ene˜8˜carboxylate (C9). Yield: 55 mg, 0.20 mmol, 50%. APCI m/z 275.0 (M+1). ¹H NMR (400 MHz, CDCl₃) δ 1.01 (d, J=6.3 Hz, 3H), 1.34 (br s, 1H), 1.57 (dd, J=14.1, 10.0 Hz, 1H). 1.75˜1.85 (m. 2H), 1.87-1.95 (m, 1H), 1.92 (d, J=1.6 Hz, 3H), 2.61 (ddd, J=12.7, 11.0, 3.3 Hz, 1H), 2.66-2.74 (m, 1H), 2.87 (ddd, J=12.6, 4.5, 4.5 Hz, 1H), 6.63 (q, J=1.5 Hz, 1H), 6.91 (ddd, J=9.5, 2.2, 2.2 Hz, 1H), 6.97 (ddd, J=7.9, 1.8, 0.9 Hz, 1H), 7.10 (dddd, J=8.4, 8.4, 2.5, 0.9 Hz, 1H), 7.39 (ddd, J=8.2, 8.1, 6.4 Hz, 1H).

Step 6. Synthesis of (5R,7S)-1-(3-fluorophenyl)-8-(4-hydroxy-3-isopropoxybenzyl)-3,7-dimethyl-1,8-diazaspiro[4,5]dec-3-en-2-one, hydrochloride salt (#98). The title product was prepared from (5R,7S)-1-(3-fluorophenyl)-3,7-dimethyl-1,8-diazaspiro[4.5]dec˜3˜en˜2˜one (#C47) according to the general procedure for the synthesis of (5R,7S)˜1˜(3˜fluorophenyl)˜8˜(4˜hydroxy˜3˜isopropoxybenzyl)˜7˜methyl˜1,8˜diazaspiro[4.5]dec˜3˜en˜2˜one hydrochloride (87) in Example 87. The neutral form of the product was obtained as an oil. Yield: 12.4 mg, 0.0283 mmol, 39%. LCMS m/z 439.6 (M+1). ¹H NMR (400 MHz, CDCl₃) δ 1.14 (d, J=6.6 Hz, 3H), 1.33 (2 d, J=6.0 Hz, 6H), 1.51˜1.56 (m, 1H), 1.62˜1.69 (m, 1H), 1.90˜1.97 (m, 1H), 1.95 (d, J=1.6 Hz, 3H), 2.07 (dd, J=13.3, 5.1 Hz, 1H), 2.38 (ddd, J=12.5, 5.7, 4.1 Hz, 1H), 2.62 (ddd, J=12.5, 9.7, 3.1 Hz, 1H), 2.92-3.00 (m, 1H), 3.44 (AB quartet, J_{AB}=13.3 Hz, Δv_{AB}=76.8 Hz, 2H), 4.52 (septet, J=6.0 Hz, 1H), 5.63 (br s, 1H), 6.68 (dd, J=8.1, 1.9 Hz, 1H), 6.77 (d, J=1.8 Hz, 1H), 6.81 (d, J=8.1 Hz, 1H), 6.88 (ddd, J=9.5, 2.2, 2.2 Hz, 1H), 6.93 (ddd, 1.8, 0.9 Hz, 1H), 7.00-7.02 (m, 1H), 7.11 (dddd, J=8.4, 8.4, 2.5, 0.8 Hz, 1H), 7.39 (ddd, J=8.1, 8.1, 6.4 Hz, 1H).

Treatment of the neutral form of the product with 1 M hydrogen chloride in diethyl ether provided the hydrochloride salt #98 as a solid, 13.2 mg.

Biological data for Examples 87-@98 is given in Table 6.

The structures of additional Examples are shown in Tables 2 and 3, which also give physical data, preparative information and biological data for these Examples.

TABLE 2
Examples #200-#212

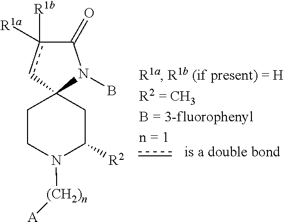

| | | | | | ¹H NMR (400 MHz, CDCl₃), δ (ppm); Mass |
|---|---|---|---|---|---|
| | | | BACE | IUPAC | spectrum: LCMS (unless otherwise |
| Ex # | A | Method | Activity¹ | Name | indicated), observed ion m/z |
| #200 | 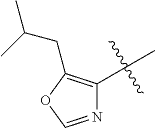 | Ex 87 | *** | (5R,7S)-1- (3- fluorophenyl)- 8-[(5- isobutyl-1,3- oxazol-4- yl)methyl]- 7-methyl- 1,8-diazaspiro [4.5]dec-3-en- 2-one, hydrochloride salt | 0.85 (d, J = 6.5 Hz, 3H), 0.86 (d, J = 6.6 Hz, 3H), 1.19 (d, J = 6.5 Hz, 3H), 1.67 (br dd, J = 14, 6 Hz, 1H), 1.80-2.01 (m, 3H), 2.09 (dd, J = 13.6, 4.7 Hz, 1H), 2.40-2.46 (m, 1H), 2.42 (d, J = 7.1 Hz, 2H), 2.66 (ddd (J = 12.4, 8.5, 3.6 Hz, 1H), 2.94-3.02 (m, 1H), 3.46 (AB quartet, J_{AB} = 14.0 Hz, Δν_{AB} = 15.0 Hz, 2H) 6.21 (d, J = 6 Hz, 1H), 6.85 (ddd, J = 9.4, 2.2, 2.2 Hz, 1H), 6.91 (ddd, J = 7.9, 1.8, 1.0 Hz, 1H), 7.08 (dddd, J = 8.4, 8.4, 2.5, 0.9 Hz, 1H), 7.32 (d, J = 6.0 Hz, 1H), 7.36 (ddd, J = 8.1, 8.1, 6.3 Hz, 1H), 7.66 (s, 1H), 398.2 (M + 1)² |
| #201 | 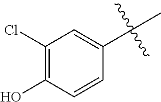 | Ex 87 | *** | (5R,7S)-8- (3-chloro-4- hydroxy- benzyl)-1-(3- fluorophenyl)- 7-methyl- 1,8-diazaspiro [4.5]dec-3-en- 2-one, hydrochloride salt | 1.14 (d, J = 6.6 Hz, 3H), 1.61 (ddd, J = 13.5, 5.1, 1.6 Hz, 1H), 1.71-1.78 (m, 1H), 1.96 (ddd, J = 13.0, 9.5, 4.0 Hz, 1H), 2.10 (dd, J = 13.4, 5.0 Hz, 1H), 2.37 (ddd, J = 12.5, 6.2, 4.1 Hz, 1H), 2.62 (ddd, J = 12.5, 9.4, 3.3 Hz, 1H), 2.91-2.98 (m, 1H), 3.43 (AB quartet, J_{AB} = 13.5 Hz, Δν_{AB} = 75.4 Hz, 2H), 6.23 (d, J = 6.0 Hz, 1H), 6.86-6.90 (m, 1H), 6.88 (d, J= 8.3 Hz, 1H), 6.94 (ddd, J = 7.9, 1.9, 1.0 Hz, 1H), 7.00 (dd, J = 8.3, 2.0 Hz, 1H), 7.12 (dddd, J = 8.4, 8.4, 2.5, 0.9 Hz, 1H), 7.18 (br d, J = 2.0 Hz, 1H), 7.39 (d, J = 6.2 Hz, 1H), 7.4-7.44 (m, 1H); APCI 401.0, 403.0 (M + 1)² |
| #202 | 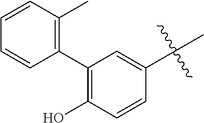 | Ex 87 | **** | (5R,7S)-1-(3- fluorophenyl)- 8-[(6-hydroxy- 2′-methyl- biphenyl-3- yl)methyl]- 7-methyl- 1,8- diazaspiro [4.5]dec-3-en- 2-one, hydrochloride salt | 1.14 (d, J = 6.6 Hz, 3H), 1.60 (ddd, J = 13.4, 4.9, 1.6 Hz, 1H), 1.70-1.76 (m, 1H), 1.97 (ddd, J = 13.1, 9.7, 4.0 Hz, 1H), 2.10 (dd, J = 13.2, 5.1 Hz, 1H), 2.14 (s, 3H), 2.42 (ddd, J = 12.4, 5.7, 4.2 Hz, 1H), 2.66 (ddd, J = 12.5, 9.6, 3.1 Hz, 1H), 2.95-3.03 (m, 1H), 3.36 (d, J = 13.2 Hz, 1H), 3.60 (d, J = 13.3 Hz, 1H), 4.90 (br s, 1H), 6.22 (d, J = 6.0 Hz, 1H), 6.87-6.91 (m, 2H), 6.95 (ddd, J = 7.8, 1.8, 0.9 Hz, 1H), 6.99 (d, J = 2.2 Hz, 1H), 7.09-7.14 (m, 2H), 7.20-7.22 (m, 1H), 7.26-7.34 (m, 3H), 7.38- 7.44 (m, 1M), 7.40 (d, J = 6.0 Hz, 1H); 457.1 (M + 1)² |
| #203 | 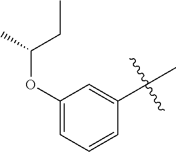 | Ex 87 | *** | (5R,7S)-1-(3- fluorophenyl)- 7-methyl- 8-(3-{[(1R)-1- methylpropyl] oxy}benzyl)- 1,8- diazaspiro[4.5] dec-3-en- 2-one, hydrochloride salt | 0.96 (t, J = 7.5 Hz, 3H), 1.15 (d, J = 6.7 Hz, 3H), 1.27 (d, J = 6.2 Hz, 3H), 1.54-1.64 (m, 2H), 1.67-1.76 (m, 2H), 1.99 (ddd, J = 12.9, 9.8, 4.1 Hz, 1H), 2.13 (dd, J = 13.3, 5.1 Hz, 1H), 2.42 (ddd, J = 12.5, 5.7, 4.2 Hz, 1H), 2.66 (ddd, J = 12.5, 9.8, 3.2 Hz, 1H), 2.97-3.05 (m, 1H), 3.38 (d, J = 13.5 Hz, 1H), 3.60 (d, J = 13.5 Hz, 1H), 4.22-4.29 (m, 1H), 6.23 (d, J = 6.2 Hz, 1H), 6.73-6.81 (m, 3H), 6.89 (ddd, J = 9.5, 2.2, 2.2 Hz, 1H), 6.95 (ddd, J = 7.9, 1.8, 1.0 Hz, 1H), 7.11-7.18 (m, 2H), 7.39-7 45 (m, 2H); 423.1 (M + 1)² |
| #204 | 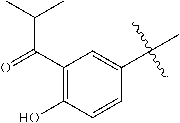 | Ex 87 | *** | (5R,7S)-1- (3- fluorophenyl)- 8-(4- hydroxy-3- isobutyryl- benzyl)-7- methyl-1,8- diazaspiro[4.5] dec-3-en- 2-one, hydrochloride salt | 1.17 (d, J = 6.7 Hz, 3H), 1.21 (br d, J = 6.8 Hz, 6H), 1.61 (ddd, J = 13.4, 4.5, 1.7 Hz, 1H), 1.70-1.76 (m, 1H), 1.98 (ddd, J = 13.1, 9.8, 4.0 Hz, 1H), 2.13 (dd, J = 13.4, 5.1 Hz, 1H), 2.40 (ddd, J = 12.5, 5.6, 4.2 Hz, 1H), 2.66 (ddd, J = 12.5, 9.8, 3.1 Hz, 1H), 2.97-3.04 (m, 1H), 3.49-3.6 (m, 1H), 3.50 (AB quartet, J_{AB} = 13.5 Hz, Δν_{AB} = 59.9 Hz, 2H), 6.24 (d, J = 6.0 Hz, 1H), 6.87-6.91 (m, 1H), 6.92 (d, J = 8.4 Hz, 1H), 6.95 (ddd, J = 7.9, 1.8, 0.9 Hz, 1H), 7.13 (dddd, J = 8.3, 8.3, 2.6, 0.9 Hz, 1H), 7.35 (dd, J = 8.5, 2.1 Hz, 1H), 7.42 (ddd, J = 8.2, 8.2. 6.3 Hz, 1H), 7.42 (d, J = 6.0 Hz, 1H), 7.62 (d, J = 2.0 Hz, 1H), 12.42 (s, 1H); 436.9 (M + 1)² |
| #205 | 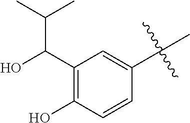 | Ex 87 | **** | (5R,7S)-1-(3- fluorophenyl)- 8-[4- hydroxy-3- (1-hydroxy-2- methylpropyl) benzyl]-7- methyl-1,8- diazaspiro[4.5] dec-3-en-2-one, hydrochloride salt | 0.79 (2 d, J = 6.8 and 6.7 Hz, 3H), 1.01 (d, J = 6.6 Hz, 3H), 1.13 (2 d, J = 6.6 Hz, 3H), 1.54-1.61 (m, 1H), 1.67-1.73 (m, 1H), 1.89- 1.98 (m, 1H), 1.99-2.11 (m, 2H), 2.31-2.41 (m, 1H), 2.56-2.65 (m, 1H), 2.88-2.99 (m, 1H), 3.05 (v br s, 1H), 3.32 (d, J = 13.1 Hz, 1H), 3.52 and 3.53 (2 d, J = 13.1 Hz, 1H), 4.40 and 4.41 (2 d, J = 7.1 Hz, 1H), 6.21 (d, J = 6.0 Hz, 1H), 6.72-6.76 (m, 2H), 6.84-6.88 (m, 1H), 6.91-6.94 (m, 1H), 6.96-7.00 (m, 1H), 7.09-7.15 (m, 1H), 7.37-7.44 (m, 2H), 8.12 (v br s, 1H); 439.0 (M + 1)² |
| #206 | 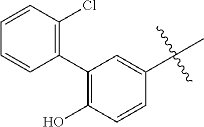 | Ex 87 | **** | (5R,7S)-8- [(2′-chloro- 6-hydroxy- biphenyl-3- yl)methyl]-1-(3- fluorophenyl)- 7-methyl-1,8- diazaspiro[4.5] dec-3-en- 2-one, hydrochloride salt | 1.14 (d, J = 6.7 Hz, 3H), 1.60 (ddd, J = 13.3, 4.9, 1.6 Hz, 1H), 1.70-1.77 (m, 1H), 1.97 (ddd, J = 13.0, 9.6, 4.1 Hz, 1H), 2.10 (dd, J = 13.4, 5.0 Hz, 1H), 2.43 (ddd, J = 12.5, 5.9, 4.1 Hz, 1H), 2.67 (ddd, J = 12.4, 9.7, 3.3 Hz, 1H), 2.95-3.03 (m, 1H), 3.49 (AB quartet, J_{AB} = 13.3 Hz, Δν_{AB} = 94.1 Hz, 2H), 5.12 (br s, 1H), 6.22 (d, J = 6.0 Hz, 1H), 6.88 (d, J = 8.3 Hz, 1H), 6.87-6.90 (m, 1H), 6.94 (ddd, J = 7.9, 1.8, 0.9 Hz, 1H), 7.05 (d, J = 2.2 Hz, 1H), 7.11 (dddd, J = 8.4, 8.4, 2.5, 0.9 Hz, 1H), 7.15 (dd, J = 8.3, 2.2 Hz, 1H), 7.31-7.43 (m, 5H), 7.50- 7.54 (m, 1H); 476.8, 479.0 (M + 1)² |
| #207 | 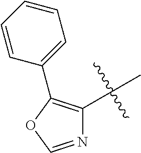 | Ex 87 | ** | (5R,7S)-1-(3- fluorophenyl)- 7-methyl- 8-[(5- phenyl-1,3- oxazol-4- yl)methyl]-1,8- diazaspiro[4.5] dec-3-en- 2-one, hydrochloride salt | 1.18 (d, J = 6.7 Hz, 3H), 1.61 (ddd, J = 13.4, 4.8, 1.7 Hz, 1H), 1.70-1.76 (m, 1H), 1.98 (ddd, J = 13.1, 9.7, 4.0 Hz, 1H), 2.12 (dd, J = 13.4, 5.0 Hz, 1H), 2.56 (ddd, J = 12.4, 5.8, 4.2 Hz, 1H), 2.74 (ddd, J = 12.4, 9.6, 3.2 Hz, 1H), 3.04-3.12 (m, 1H), 3.70 (AB quartet, J_{AB} = 13.6 Hz, Δν_{AB} = 71.2 Hz, 2H), 6.22 (d, J = 6.2 Hz, 1H), 6.86 (ddd, J = 9.4, 2.2, 2.2 Hz, 1H), 6.92 (ddd, J = 7.9, 1.8, 0.9 Hz, 1H), 7.12 (dddd, J = 8.4, 8.4, 2.5, 0.9 Hz, 1H), 7.31-7.43 (m, 5H), 7.64-7.68 (m, 2H), 7.82 (s, 1H); 417.9 (M + 1)² |
| #208 | 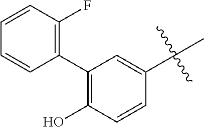 | Ex 87 | **** | (5R,7S)-8- [(2′-fluoro-6- hydroxy- biphenyl-3- yl)methyl]-1-(3- fluorophenyl)- 7-methyl-1,8- diazaspiro[4.5] dec-3-en-2-one, hydrochloride salt | 1.15 (d, J = 6.6 Hz, 3H), 1.62 (br dd, J = 13, 5 Hz, 1H), 1.72-178 (br m, 1H), 1.93-2.00 (m, 1H), 2.09 (dd, J = 13.5, 4.9 Hz, 1H), 2.38-2.45 (m, 1H), 2.63-2.70 (m, 1H), 2.94-3.01 (m, 1H), 3.50 (AB quartet, J_{AB} = 13.3 Hz, Δν_{AB} = 83.3 Hz, 2H), 6.22 (d, J = 6.0 Hz, 1H), 6.85-6.90 (m, 2H), 6.93 (br d, J = 8 Hz, 1H), 7.07-7.26 (m, 5H), 7.33-7.42 (m, 4H); 461.5 (M + 1)² |
| #209 | 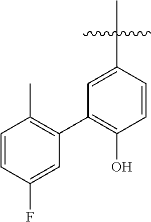 | Ex 87 | **** | (5R,7S)-8- [(5′-fluoro-6- hydroxy-2′- methyl- biphenyl-3- yl)methyl]-1-(3- fluorophenyl)- 7-methyl-1,8- diazaspiro[4.5] dec-3-en- 2-one, hydrochloride salt | 1.14 (d, J = 6.7 Hz, 3H), 1.60 (ddd, J = 13.4, 4.9, 1.5 Hz, 1H), 1.70-1.77 (m, 1H), 1.97 (ddd, J = 13.0, 9.6, 4.0 Hz, 1H), 2.09 (s, 3H), 2.10 (dd, J = 13.3, 5.0 Hz, 1H), 2.41 (ddd, J = 12.4, 5.9, 4.1 Hz, 1H), 2.66 (ddd, J = 12.4, 9.5, 3.1 Hz, 1H), 2.94-3.02 (m, 1H), 3.43 (AB quartet, J_{AB} = 13.3 Hz, Δν_{AB} = 96.1 Hz, 2H), 5.13 (br s, 1H), 6.22 (d, J = 6.2 Hz, 1H), 6.86 (d, J = 8.3 Hz, 1H), 6.88 (ddd, J = 9.4, 2.2, 2.2 Hz, 1H), 6.91-6.96 (m, 3H), 7.01 (ddd, J = 8.4, 8.4, 2.8 Hz, 1H), 7.09-7.14 (m, 2H), 7.26 (dd, J = 8.5, 5.8 Hz, 1H), 7.40 (d, J = 6.1 Hz, 1H), 7.41 (ddd, J = 8.2, 8.2, 6.4 Hz, 1H); 474.9 (M + 1)² |
| #210 | 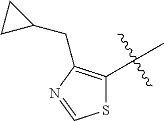 | Ex 87 | **** | (5R,7S)-8-{[4- (cyclopropyl- methyl)-1,3- thiazol-5- yl]methyl}- 1-(3- fluorophenyl)- 7-methyl-1,8- diazaspiro[4.5] dec-3-en- 2-one, hydrochloride salt | 0.14-0.18 (m, 2H), 0.43-0.48 (m, 2H), 0.97- 1.07 (m, 1H), 1.16 (d, J = 6.7 Hz, 3H), 1.60 (ddd, J = 13.4, 4.8, 1.7 Hz, 1H), 1.70-1.77 (m, 1H), 1.97 (ddd, J = 13.1, 9.6, 4.0 Hz, 1H), 2.11 (dd, J = 13.4, 5.0 Hz, 1H), 2.46 (ddd, J = 12.4, 5.8, 4.1 Hz, 1H), 2.60 (d, J = 6.7 Hz, 2H), 2.68 (ddd, J = 12.5, 9.6, 3.2 Hz, 1H), 3.02-3.10 (m, 1H), 3.67 (AB quartet, J_{AB} = 14.2 Hz, Δν_{AB} = 82.8 Hz, 2H), 6.24 (d, J = 6.0 Hz, 1H), 6.89 (ddd, J = 9.3, 2.2, 2.2 Hz, 1H), 6.95 (ddd, J = 7.9, 1.8, 0.9 Hz, 1H), 7.14 (dddd, J = 8.4, 8.4, 2.5, 0.9 Hz, 1H), 7.39 (d, J = 6.0 Hz, 1H), 7.43 (ddd, J = 8.1, 8.1, 6.3 Hz, 1H), 8.63 (s, 1H); 411.9 (M + 1)² |
| #211 | 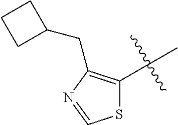 | Ex 87 | **** | (5R,7S)-8-{[4- (cyclobutyl methyl)-1,3- thiazol-5- yl]methyl}- 1-(3- fluorophenyl)- 7-methyl-1,8- diazaspiro[4.5] dec-3-en-2-one, hydrochioride salt | 1.17 (d, J = 6.6 Hz, 3H), 1.57-2.04 (m, 9H), 2.11 (dd, J = 13.5, 5.1 Hz, 1H), 2.46 (ddd, J = 12.4, 5.7, 4.2 Hz, 1H), 2.61-2.75 (m, 4H), 3.02-3.10 (m, 1H), 3.68 (AB quartet, J_{AB} = 14.2 Hz, Δν_{AB} = 82.2 Hz, 2H), 6.24 (d, J = 6.1 Hz, 1H), 6.89 (ddd, J = 9.4, 2.2, 2.2 Hz, 1H); 6.95 (ddd, J = 7.9, 1.8, 0.9 Hz, 1H), 7.14 (dddd, 7 = 8.4, 8.4, 2.5, 1.0 Hz, 1H), 7.4-7.45 (m, 1H), 7.41 (d, J = 6.1 Hz, 1H), 8.59 (s, 1H); 425.9 (M + 1)² |
| #212 | 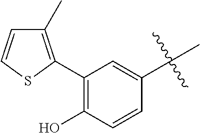 | Ex 87 | **** | (5R,7S)-1- (3- fluorophenyl)- 8-[4- hydroxy-3- (3-methyl-2- thienyl)benzyl]- 7-methyl-1,8- diazaspiro[4.5] dec-3-en- 2-one, hydrochloride salt | 1.14 (d, J = 6.7 Hz, 3H), 1.61 (ddd, J = 13.4, 4.9, 1.3 Hz, 1H), 1.71-1.77 (m, 1H), 1.96 (ddd, J = 13.0, 9.5, 4.0 Hz, 1H), 2.06-2.1 (m, 1H), 2.11 (s, 3H), 2.41 (ddd, J = 12.5, 6.0, 4.1 Hz, 1H), 2.66 (ddd, J = 12.4, 9.5, 3.2 Hz, 1H), 2.94-3.02 (m, 1H), 3.47 (AB quartet, J_{AB} = 13.3 Hz, Δν_{AB} = 76.3 Hz, 2H), 6.22 (d, J = 6.1 Hz, 1H), 6.81-6.84 (m, 1H), 6.88 (ddd, J = 9.4, 2.2, 2.2 Hz, 1H), 6.92-6.95 (m, 1H), 6.95 (d, J = 5.2 Hz, 1H), 7.07-7.12 (m, 3H), 7.29 (d, J = 5.1 Hz, 1H), 7.39 (ddd, J = 8.1, 8.1, 6.3 Hz, 1H), 7.40 (d, J = 6.1 Hz, 1H); 463.4 (M + 1)² |

¹BACE activity Cell Free Assay IC₅₀ 1 nM to 1 μM ****, 1 μM to 10 μM ***, 10 μM to 100 μM **, 100 μM to 300 μM *
²NMR and MS data obtained on free base, prior to formation of hydrochloride salt.

TABLE 3
Examples #213-#217

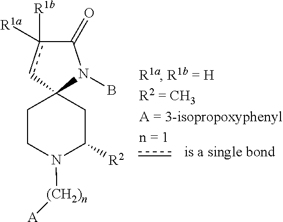

| | | | | | ¹H NMR (400 MHz, CDCl₃), δ (ppm); Mass |
|---|---|---|---|---|---|
| | | | BACE | IUPAC | spectrum: LCMS (unless otherwise |
| Ex # | A | Method | Activity¹ | Name | indicated), observed ion m/z |
| #213 | 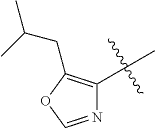 | Ex 88 | ** | (5R,7S)-1-(3- fluorophenyl)- 8-[(5-isobutyl- 1,3-oxazol-4- yl)methyl]-7- methyl-1,8- diazaspiro[4.5] decan-2-one, hydrochloride salt | 0.84 (d, J = 6.6 Hz, 3H), 0.85 (d, J = 6.7 Hz, 3H), 1.13 (d, J = 6.8 Hz, 3H), 1.54-1.59 (m, 1H), 1.65-1.72 (m, 1H), 1.85-1.95 (m, 2H), 2.02-2.12 (m, 2H), 2.25 (ddd, J = 12.6, 8.6, 4.6 Hz, 1H), 2.4-2.47 (m, 1H), 2.42 (d, J = 7.0 Hz, 2H), 2.53-2.63 (m, 3H), 2.94- 3.01 (m, 1H), 3.40 (s, 2H), 6.81 (ddd, J = 9.4, 2.2, 2.2 Hz, 1H), 6.87 (ddd, J = 7.9, 1.8, 0.9 Hz, 1H), 7.05 (dddd, J = 8.4, 8.4, 2.5, 0.9 Hz, 1H), 7.34 (ddd, J = 8.2, 8.1, 6.3 Hz, 1H), 7.66 (s, 1H); 400.2 (M + 1)² |
| #214 | 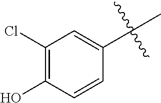 | Ex 88 | *** | (5R,7S)-8-(3- chloro-4- hydroxybenzyl)- 1-(3- fluorophenyl)- 7-methyl-1,8- diazaspiro[4.5] decan-2-one, hydrochloride salt | 1.10 (d, J = 6.9 Hz, 3H), 1.57 (ddd, J = 13.2, 4.0, 2.1 Hz, 1H), 1.64-1.70 (m, 1H), 1.84 (br ddd, J = 13, 10.4 Hz, 1H), 2.00 (dd, J = 13.2, 5.3 Hz, 1H), 2.10 (br ddd, J = 12.5, 9.4, 9.4 Hz, 1H), 2.27 (ddd, J = 12.6, 8.6, 4.2 Hz, 1H), 2.38 (br ddd, J = 12.4, 4.6, 4.6 Hz, 1H), 2.50-2.65 (m, 3H), 2.90-2.98 (m, 1H), 3.40 (AB quartet, J_{AB} = 13.4 Hz, Δν_{AB} = 33.5 Hz, 2H), 5.67 (v br s, 1H), 6.84 (ddd, J = 9.4, 2.2, 2.2 Hz, 1H), 6.89 (d, J = 8.3 Hz, 1H), 6.90 (ddd, J = 7.9, 1.9, 1.0 Hz, 1H), 7.01 (dd, J = 8.3, 2.0 Hz, 1H), 7.10 (dddd, J = 8.4, 8.4, 2.5, 0.9 Hz, 1H), 7.18 (d, J = 2.0 Hz, 1H), 7.39 (ddd, J = 8.1, 8.1, 6.3 Hz, 1H); 403.1, 405.1 (M + 1)² |
| #215 | 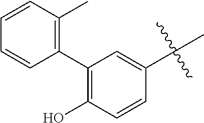 | Ex 88 | **** | (5R,7S)-1-(3- fluorophenyl)- 8-[(6-hydroxy- 2′- methylbiphenyl- 3-yl)methyl]-7- methyl-1,8- diazaspiro[4.5] decan-2-one, hydrochloride salt | 1.10 (d, J = 6.9 Hz, 3H), 1.57 (ddd, J = 13, 4, 2 Hz, 1H) 1.64-1.70 (m, 1H), 1.81-1.89 (m, 1H), 2.00 (dd, J = 13.0, 5.4 Hz, 1H), 2.06-2.14 (m, 1H), 2.14 (s, 3H), 2.28 (ddd, J = 12.5, 8.6, 4.2 Hz, 1H), 2.44 (br ddd, J = 12.5, 4.5, 4.5 Hz, 1H), 2.50-2.65 (m, 3H), 2.95-3.01 (m, 1H), 3.45 (br AB quartet, J_{AB} = 13 Hz, Δν_{AB} = 46 Hz, 2H), 6.84 (ddd, J = 9.4, 2.2, 2.2 Hz, 1H), 6.89 (d, J = 8.3 Hz, 1H), 6.90 (ddd, J = 7.9, 1.8, 0.9 Hz, 1H), 6.98 (d, J = 2.2 Hz, 1H), 7.08-7.14 (m, 2H), 7.20-7.22 (m, 1H), 7.26-7.34 (m, 3H), 7.38 (ddd, J = 8.2, 8.1, 6.3 Hz, 1H); 459.1 (M + 1)² |
| #216 | 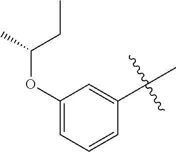 | Ex 88 | *** | (5R,7S)-1-(3- fluorophenyl)- 7-methyl-8-(3- {[(1R)-1- methylpropyl] oxy}benzyl)-1,8- diazaspiro[4.5] decan-2-one, hydrochloride salt | 0.95 (t, J = 7.5 Hz, 3H), 1.11 (d, J = 6.9 Hz, 3H), 1.26 (d, J = 6.0 Hz, 3H), 1.54-1.78 (m, 4H), 1.82-1.89 (m, 1H), 2.02 (dd, J = 13.1, 5.5 Hz, 1H), 2.06-2.15 (m, 1H), 2.29 (ddd, J = 12.6, 8.6, 4.1 Hz, 1H), 2.43 (ddd, J = 12.4, 4.5, 4.5 Hz, 1H), 2.50-2.65 (m, 3H), 2.96-3.04 (m, 1H), 3.47 (AB quartet, J_{AB} = 13.6 Hz, Δν_{AB} = 39.0 Hz, 2H), 4.21- 4.28 (m, 1H), 6.73-6.80 (m, 3H), 6.85 (ddd, J = 9.4, 2.2, 2.2 Hz, 1H), 6.91 (ddd, J = 7.9, 1.9, 0.9 Hz, 1H), 7.10 (dddd, J = 8.4, 8.4, 2.5, 1.0 Hz, 1H), 7.16 (br dd, J = 8, 8 Hz, 1H), 7.40 (ddd, J = 8.2, 8.1, 6.3 Hz, 1H); 425.2 (M + 1)² |
| #217 | 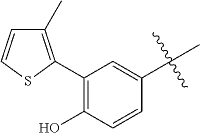 | Ex 88 | *** | (5R,7S)-1-(3- fluorophenyl)- 8-[4-hydroxy-3- (3-methyl-2- thienyl)benzyl]- 7-methyl-1,8- diazaspiro[4.5] decan-2-one, hydrochloride salt | 1.11 (d, J = 6.8 Hz, 3H), 1.55-1.60 (m, 1H), 1.65-171 (m, 1H), 1.81-1.89 (m, 1H), 2.00 (dd, J = 13.2, 5.4 Hz, 1H), 2.06-2.15 (m, 1H), 2.11 (s, 3H), 2.28 (ddd, J = 12.5, 8.7, 4.2 Hz, 1H), 2.43 (ddd, J = 12.3, 4.5, 4.5 Hz, 1H), 2.50-2.65 (m, 3H), 2.94-3.02 (m, 1H), 3.45 (AB quartet, J_{AB} = 13.4 Hz, Δν_{AB} = 38.8 Hz, 2H), 6.84 (ddd, J = 9.4, 2.2, 2.2 Hz, 1H), 6.89 (d, J = 8.3 Hz, 1H), 6.90 (ddd, J = 7.8, 1.9, 0.9 Hz, 1H), 6.98 (d, J = 5.2 Hz, 1H), 7.07-7.12 (m, 2H), 7.14 (dd, J = 8.3, 2.2 Hz, 1H), 7.33 (d, J = 5.2 Hz, 1H), 7.39 (ddd, J = 8.1, 8.1, 6.4 Hz, 1H)² |

¹BACE activity Cell Free Assay IC₅₀ 1 nM to1 μM ****, 1 μM to 10 μM ***, 10 μM to 100 μM**, 100 μM to 300 μM*
²NMR and MS data obtained on free base, prior to formation of hydrochloride salt.

### Examples #101-#126

### 1-Heteroaryl-substituted (5R,7S)-8-(3-isopropoxybenzyl)-7-methyl-1,8-diazaspiro[4.5]dec˜3˜en˜2˜ones

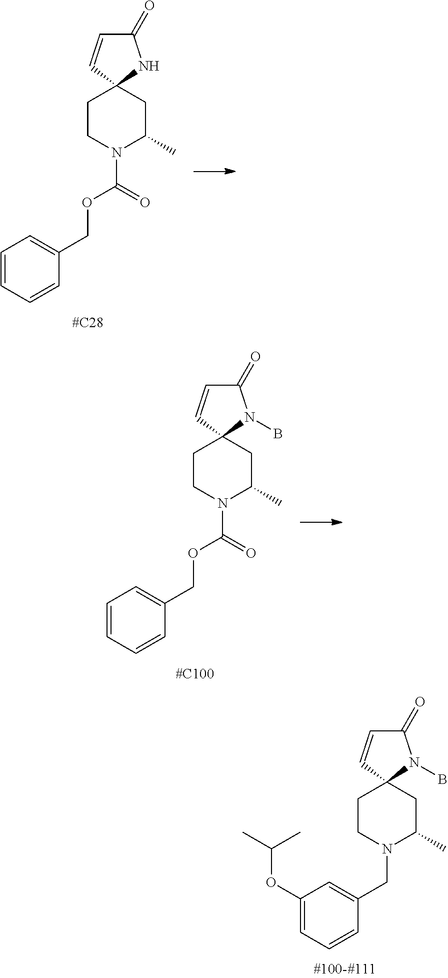

Step 1. Synthesis of 1-heteroaryl-substituted benzyl (5R,7S)-7-methyl-2-oxo-1,8-diazaspiro[4.5]dec˜3˜ene˜8˜carboxylate (#C100). A solution of benzyl (5R,7S)˜7˜methyl-2-oxo-1,8-diazaspiro[4.5]dec-3-ene-8-carboxylate (#C28) (45 mg, 0.15 mmol) and N,N′˜dimethylethylenediamine (5 equivalents) in dioxane (0.92 mL) was added to a mixture of the heteroaryl iodide or bromide (3 equivalents), copper(I) iodide (4 equivalents) and either cesium carbonate or potassium phosphate (3 equivalents) in a 1˜dram vial. The resulting suspension was sealed and heated at 80˜90° C. for 18˜66 hours. The reaction mixture was then cooled to room temperature, diluted with ethyl acetate, and flushed through an MCX cartridge containing a small amount of Celite on top of the packing material. Additional ethyl acetate (5˜10 mL) was eluted through the cartridge, and the combined filtrates were concentrated in vacuo to afford the product, which was taken directly into the next step.

Step 2. Synthesis of 1-heteroaryl-substituted (5R,7S)-8-(3-isopropoxybenzyl)˜7˜methyl˜1,8˜diazaspiro[4,5]dec˜3˜en˜2˜ones (#101-#126). The 1˜heteroaryl-substituted benzyl (5R,7S)-7-methyl-2-oxo-1,8-diazaspiro[4.5]dec-3-ene-8-carboxylate (#C100) from the previous step was dissolved in a freshly prepared solution of trimethylsilyl iodide (0.3 M in acetonitrile, 2 equivalents), and the resulting solution was stirred at room temperature for 18 hours. Purification was carried out by loading the reaction mixture directly onto an MCX column. The column was flushed with dichloromethane (5 mL), and the product was then eluted using a 2 M solution of ammonia in methanol (5 mL). The eluant was concentrated in vacuo to afford the deprotected intermediate. This material was mixed with acetonitrile (1 mL) and potassium carbonate (3 equivalents). After addition of 1˜(bromomethyl)˜3˜isopropoxybenzene (2 equivalents), the mixture was stirred at room temperature for 18 hours, then loaded onto an MCX cartridge containing a small amount of Celite on top of the packing material. The cartridge was flushed with dichloromethane (5 mL), and the filtered solids and Celite were manually removed from the cartridge. The product was eluted using a 2 M solution of ammonia in methanol (5 mL), and the filtrate was concentrated in vacuo.

Purification was carried by preparative HPLC using one of the following systems: 1) Column: Waters XBridge C₁₈, 5 μm; Mobile phase A: 0.03% NH₄OH in water (v/v); Mobile phase B: 0.03% NH₄OH in acetonitrile (v/v); Gradient: 5-30% B to 100% B; or 2) Column: Waters Sunfire C₁₈, 5 μm; Mobile phase A: 0.05% trifluoroacetic acid in water (v/v); Mobile phase B: 0.05% trifluoroacetic acid in acetonitrile (v/v); Gradient: 10 or 15% to 100% B. See Table 4 for characterization data and biological activity.

TABLE 4
Examples #101-#126

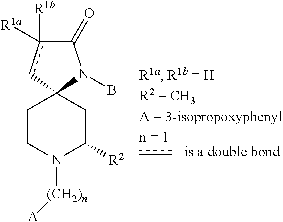

| | | | | Retention | Mass |
|---|---|---|---|---|---|
| | | | | Time (min) | spectrum: |
| | | | | [HPLC | Observed |
| | | BACE | | method in | ion m/z |
| Ex # | B | Activity¹ | IUPAC Name | footnotes] | (M + 1) |
| #101 | 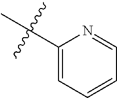 | ** | (5R,7S)-8-(3-isopropoxybenzyl)-7- methyl-1-pyridin-2-yl-1,8- diazaspiro[4.5]dec-3-en-2-one | 2.48 | 392.2 |
| #102 | 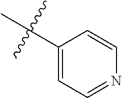 | * | (5R,7S)-8-(3-isopropoxybenzyl)-7- methyl-1-pyridin-4-yl-1,8- diazaspiro[4.5]dec-3-en-2-one | 2.40 | 392.3 |
| #104 | 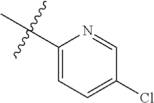 | ** | (5R,7S)-1-(5-chloropyridin-2-yl)-8-(3- isopropoxybenzyl)-7-methyl-1,8- diazaspiro[4.5]dec-3-en-2-one, trifluoroacetate salt | 2.44 | 426.2, 428.2 |
| #105 | 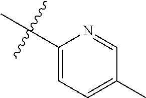 | ** | (5R,7S)-8-(3-isopropoxybenzyl)-7- methyl-1-(5-methylpyridin-2-yl)-1,8- diazaspiro[4.5]dec-3-en-2-one, trifluoroacetate salt | 2.13 | 406.3 |
| #106 | 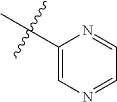 | ** | (5R,7S)-8-(3-isopropoxybenzyl)-7- methyl-1-pyrazin-2-yl-1,8- diazaspiro[4.5]dec-3-en-2-one, trifluoroacetate salt | 2.08 | 393.3 |
| #107 | 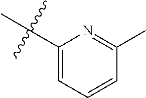 | *** | (5R,7S)-8-(3-isopropoxybenzyl)-7- methyl-1-(6-methylpyridin-2-yl)-1,8- diazaspiro[4.5]dec-3-en-2-one | 2.09 | 406.3 |
| #108 | 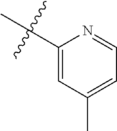 | *** | (5R,7S)-8-(3-isopropoxybenzyl)-7- methyl-1-(4-methylpyridin-2-yl)-1,8- diazaspiro[4.5]dec-3-en-2-one | 2.12 | 406.3 |
| #109 | 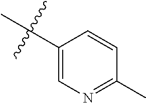 | ** | (5R,7S)-8-(3-isopropoxybenzyl)-7- methyl-1-(6-methylpyridin-3-yl)-1,8- diazaspiro[4.5]dec-3-en-2-one | 1.81 | 406.3 |
| #110 | 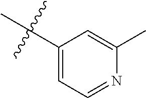 | ** | (5R,7S)-8-(3-isopropoxybenzyl)-7- methyl-1-(2-methylpyridin-4-yl)-1,8- diazaspiro[4.5]dec-3-en-2-one | 1.66 | 406.3 |
| #111 | 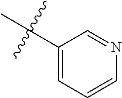 | ** | (5R,7S)-8-(3-isopropoxybenzyl)-7- methyl-1-pyridin-3-yl-1,8- diazaspiro[4.5]dec-3-en-2-one | 1.81 | 392.2 |
| #112 | 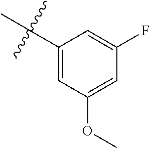 | *** | (5R,7S)-1-(3-fluoro-5-methoxyphenyl)- 8-(3-isopropoxybenzyl)-7-methyl-1,8- diazaspiro[4.5]dec-3-en-2-one | 2.37 | 439.2 |
| #113 | 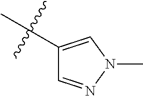 | ** | (5R,7S)-8-(3-isopropoxybenzyl)-7- methyl-1-(1-methyl-1H-pyrazol-4-yl)- 1,8-diazaspiro[4.5]dec-3-en-2-one | 1.87 | 395.3 |
| #114 | 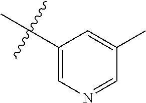 | *** | (5R,7S)-8-(3-isopropoxybenzyl)-7- methyl-1-(5-methylpyridin-3-yl)-1,8- diazaspiro[4.5]dec-3-en-2-one | 1.89 | 406.2 |
| #115 | 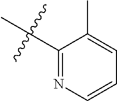 | ** | (5R,7S)-8-(3-isopropoxybenzyl)-7- methyl-1-(3-methylpyridin-2-yl)-1,8- diazaspiro[4.5]dec-3-en-2-one | 1.97 | 406.3 |
| #116 | 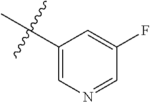 | *** | (5R,7S)-1-(5-fluoropyridin-3-yl)-8-(3- isopropoxybenzyl)-7-methyl-1,8- diazaspiro[4.5]dec-3-en-2-one | 2.02 | 410.2 |
| #117 | 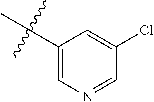 | *** | (5R,7S)-1-(5-chloropyridin-3-yl)-8-(3- isopropoxybenzyl)-7-methyl-1,8- diazaspiro[4.5]dec-3-en-2-one | 2.14 | 426.2, 428.2 |
| #118 | 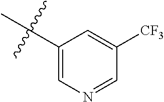 | ** | (5R,7S)-8-(3-isopropoxybenzyl)-7- methyl-1-[5-(trifluoromethyl)pyridin-3- yl]-1,8-diazaspiro[4.5]dec-3-en-2-one | 2.34 | 460.2 |
| #119 | 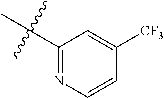 | * | (5R,7S)-8-(3-isopropoxybenzyl)-7- methyl-1-[4-(trifluoromethyl)pyridin-2- yl]-1,8-diazaspiro[4.5]dec-3-en-2-one | 2.79 | 460.1 |
| #120 | 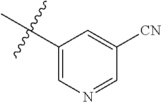 | ** | 5-{(5R,7S)-8-(3-isopropoxybenzyl)-7- methyl-2-oxo-1,8-diazaspiro[4.5]dec-3- en-1-yl]nicotinonitrile | 2.10 | 417.2 |
| #121 | 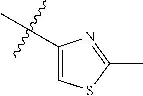 | *** | (5R,7S)-8-(3-isopropoxybenzyl)-7- methyl-1-(2-methyl-1,3-thiazol-4-yl)- 1,8-diazaspiro[4.5]dec-3-en-2-one | 2.10 | 412.1 |
| #122 | 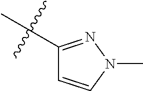 | ** | (5R,7S)-8-(3-isopropoxybenzyl)-7- methyl-1-(1-methyl-1H-pyrazol-3-yl)- 1,8-diazaspiro[4.5]dec-3-en-2-one | 1.93 | 395.2 |
| #123 | 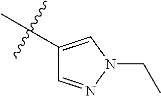 | * | (5R,7S)-1-(1-ethyl-1H-pyrazol-4-yl)-8- (3-isopropoxybenzyl)-7-methyl-1,8- diazaspiro[4.5]dec-en-2-one | 1.97 | 409.2 |
| #124 | 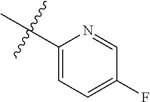 | ** | (5R,7S)-1-(5-fluoropyridin-2-yl)-8-(3- isopropoxybenzyl)-7-methyl-1,8- diazaspiro[4.5]dec-3-en-2-one, trifluoroacetate salt | 2.25 | 410.2 |
| #125 | 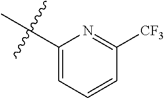 | ** | (5R,7S)-8-(3-isopropoxybenzyl)-7- methyl-1-[6-(trifluoromethyl)pyridin-2- yl]-1,8-diazaspiro[4.5]dec-3-en-2-one, trifluoroacetate salt | 2.70 | 460.2 |
| #126 | 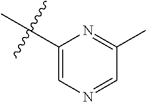 | ** | (5R,7S)-8-(3-isopropoxybenzyl)-7- methyl-1-(6-methylpyrazin-2-yl)-1,8- diazaspiro[4.5]dec-3-en-2-one, trifluoroacetate salt | 2.22 | 407.2 |

```
¹BACE activity Cell Free Assay IC₅₀: 1 nM to 1 μM ****, 1 μM to 10 μM ***, 10 μM to 100 μM**, 100 μM to 300 μM*
²Column: Waters Atlantis dC₁₈, 4.6 × 50 mm, 5 μm; Mobile phase A: 0.05% TFA in water (v/v); Mobile phase B: 0.05% TFA in acetonitrile (v/v); Flow rate: 2.0 mL/min;
Gradient:
  0 minutes  5% B
4.0 minutes 95% B
5.0 minutes 95% B
³Column: Waters XBridge C₁₈, 4.6 × 50 mm, 5 μm; Mobile phase A: 0.03% NH₄OH in water (v/v); Mobile phase B: 0.03% NH₄OH in acetonitrile (v/v); Flow rate: 2.0 mL/min;
Gradient:
  0 minutes 15% B
4.0 minutes 95% B
5.0 minutes 95% B
```

### Examples #130-#141

### 1-Heteroaryl-8-substituted-5R,7S)-7-methyl-1,8-diazaspiro[4.5]dec-3-en-2-ones

These compounds were prepared from benzyl (5R,7S)-7-methyl-2-oxo-1,8-diazaspiro[4.5]dec-3-ene-8-carboxylate (#C28) in a manner analogous to the preparation of Examples #101-#126, except that the 8-substituent was introduced via reductive amination (see Examples 1˜87). Purification was carried out by preparative HPLC using the same systems described for Examples #101-#126. See Table 5 for characterization data and biological activity.

TABLE 5
Examples #130-#141

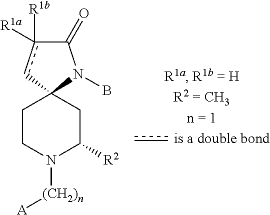

| | | | | | Retention | Mass |
|---|---|---|---|---|---|---|
| | | | | | Time (min) | spectrum: |
| | | | | | [HPLC | Observed |
| | | | BACE | | method in | ion m/z |
| Ex # | A | B | Activity¹ | IUPAC Name | footnotes] | (M + 1) |
| #130 | 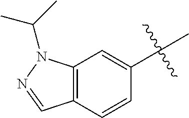 | 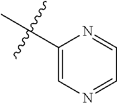 | ^{**} | (5R,7S)-8-[(1-isopropyl-1H- indazol-6-yl)methyl]-7-methyl-1- pyrazin-2-yl-1,8- diazaspiro[4.5]dec-3-en-2-one | 2.30 | 417.0 |
| #131 | 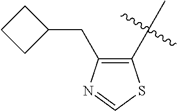 | 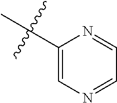 | ^{***} | (5R,7S)-8-([4-(cyclobutylmethyl)- 1,3-thiazol-5-yl]methyl)-7- methyl-1-pyrazin-2-yl-1,8- diazaspiro[4.5]dec-3-en-2-one | 2.47 | 410.0 |
| #132 | 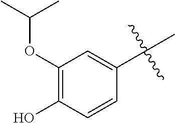 | 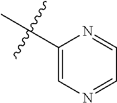 | ^{****} | (5R,7S)-8-(4-hydroxy-3- isopropoxybenzyl)-7-methyl-1- pyrazin-2-yl-1,8- diazaspiro[4.5]dec-3-en-2-one | 1.95 | 409.0 |
| #133 | 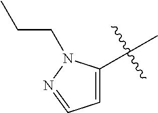 | 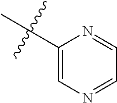 | ^{*} | (5R,7S)-7-methyl-8-[(1-propyl- 1H-pyrazol-5-yl)methyl]-1- pyrazin-2-yl-1,8- diazaspiro[4.5]dec-3-en-2-one | 2.02 | 367.1 |
| #134 | 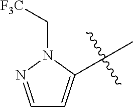 | 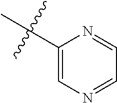 | ^{*} | (5R,7S)-7-methyl-1-pyrazin-2-yl- 8-[(1-(2,2,2-trifluoroethyl)-1H- pyrazol-5-yl]methyl]-1,8- diazaspiro[4.5]dec-3-en-2-one | 2.30 | 407.0 |
| #135 | 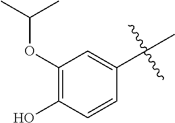 | 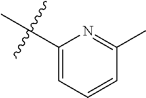 | ^{****} | (5R,7S)-8-(4-hydroxy-3- isopropoxybenzyl)-7-methyl-1- (6-methylpyridin-2-yl)-1,8- diazaspiro[4.5]dec-3-en-2-one | 2.10 | 422.2 |
| #136 | 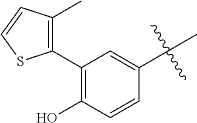 | 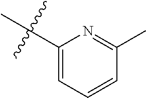 | ^{****} | (5R,7S)-8-[4-hydroxy-3-(3- methyl-2-thienyl)benzyl]-7- methyl-1-(6-methylpyridin-2-yl)- 1,8-diazaspiro[4.5]dec-3-en-2- one | 2.48 | 460.1 |
| #137 | 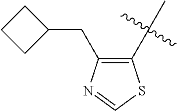 | 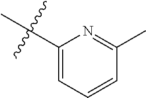 | ^{***} | (5R,7S)-8-{[4-(cyclobutylmethyl)- 1,3-thiazol-5-yl]methyl}-7- methyl-1-(6-methylpyridin-2-yl)- 1,8-diazaspiro[4.5]dec-3-en-2- one | 2.53 | 423.2 |
| #138 | 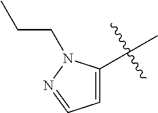 | 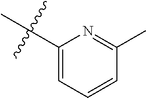 | ^{**} | (5R,7S)-7-methyl-1-(6- methylpyridin-2-yl)-8-[(1-propyl- 1H-pyrazol-5-yl)methyl]-1,8- diazaspiro[4.5]dec-3-en-2-one | 2.05 | 380.2 |
| #139 | 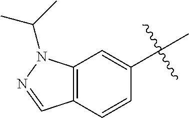 | 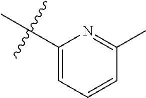 | ^{*} | (5R,7S)-8-[(1-isopropyl-1H- indazol-6-yl)methyl]-7-methyl-1- (6-methylpyridin-2-yl)-1,8- diazaspiro[4.5]dec-3-en-2-one | 2.41 | 430.2 |
| #140 | 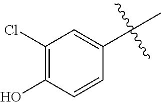 | 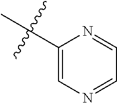 | ^{**} | (5R,7S)-8-(3-chloro-4- hydroxybenzyl)-7-methyl-1- pyrazin-2-yl-1,8- diazaspiro[4.5]dec-3-en-2-one, ammonium salt | 1.57 | 385.0, 386.9 |
| #141 | 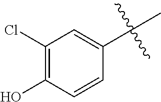 | 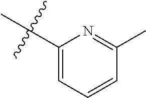 | ^{***} | (5R,7S)-8-(3-chloro-4- hydroxybenzyl)-7-methyl-1-(6- methylpyridin-2-yl)-1,8- diazaspiro[4.5]dec-3-en-2-one, ammonium salt | 1.62 | 398.1, 400.1 |

¹BACE activity Cell Free Assay IC₅₀ 1 nM to 1 μM****, 1 μM to 10 μM***, 10 μM to 100 μM**, 100 μM to 300 μM*

TABLE 6
Biological Data for Examples **

| Example | |
|---|---|
| Number | BACE Activity¹ |
| 87 | **** |
| 88 | *** |
| 89 | **** |
| 90 | ** |
| 91 | ** |
| 92 | ** |
| @93 | * |
| @94 | * |
| @95 | * |
| @96 | ** |
| @97 | *** |
| @98 | **** |

¹BACE activity Cell Free Assay IC₅₀ 1 nM to 1 μM ****, 1 μM to 10 μM ***, 10 μM to 100 μM **, 100 μM to 300 μM *

### Biological Assay

A synthetic APP substrate that can be cleaved by beta˜secretase and having N-terminal biotin is used to assay beta-secretase activity in the presence or absence of the inhibitory compounds. The substrate can contain either the wildtype sequence around the BACE cleavage site or the Swedish mutation (Vassar, R., B. D. Bennett et al. (1999). “beta˜secretase cleavage of Alzheimer's amyloid precursor protein by the transmembrane aspartic protease BACE.” Science. 286(5440): 735˜741). The substrate and test compounds are added to 384 well polypropylene plates. The reaction is initiated by the addition of soluble BACE enzyme to a final volume of 12.5 μL per well. The final assay conditions are: 0.001˜300 μM compound inhibitor, 0.05M sodium acetate (pH 4.5), 3 μM substrate, soluble human BACE, and 2% DMSO. Concentrated conditioned media from cells secreting human recombinant soluble BACE was titrated to provide a source of BACE enzyme. The cell media can be used as either a crude BACE prep or BACE can be purified using any number of techniques, including immobilized BACE inhibitor purification columns. The assay mixture is incubated for 1 hour at 37° C., and the reaction is quenched by the addition of an equal volume of 0.1M Tris, pH 8. Half of the quenched mix is incubated on clear streptavidin coated 384 well polystyrene plates for 1 hour. An ELISA is then performed using an in˜house antibody that specifically recognizes the new C˜terminus created after cleavage by BACE. Two in-house antibodies are available; each is cleavage specific, but one is raised against the wildtype sequence (APP 591-596) while the other is raised against the swedish mutation (APP 590-596). (These polyclonal antibodies were raised in rabbits by immunizing with antigen comprised of six amino acid residues present at the carboxy terminus of the wild-type soluble APPbeta sequence (NH2-ISEVKM-COOH) or seven amino acid residues present at the carboxy terminus of the Swedish mutation at the beta cleavage site (NH2-EISEVNL˜COOH) conjugated to keyhole limpet hemacyanin by methods known to those skilled in the art.) A secondary anti˜species Horseradish Peroxidase (HRP) conjugated antibody is then utilized. The readout, following assay development with TMB substrate and quenching with 0.09M sulfuric acid, is absorbance at 450 nm.

We claim:

1. A compound of formula I: 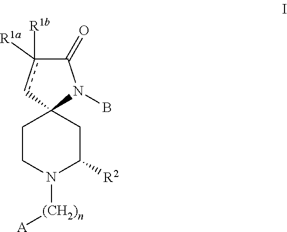
wherein the stereochemistry shown in formula I at the carbon bonded to R² and at the spirocyclic carbon is the absolute stereochemistry; B is alkyl, aryl, heteroaryl, cycloalkyl, or heterocycloalkyl, wherein B is optionally substituted with zero to three R³ groups;
A is independently aryl, cycloalkyl, heterocycloalkyl or heteroaryl wherein said aryl, cycloalkyl, heterocycloalkyl or heteroaryl is optionally substituted with one to three R⁴;
when [symbol] is a single bond, R^{1a} and R^{1b} are each independently hydrogen, alkyl, alkenyl, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, —(CH₂)ₜ—OR⁵, —(CH₂)ₜN(R⁷)₂, —NH—(CH₂)ₜ-cycloalkyl, —NH—(CH₂)ₜ-heterocycloalkyl, —NH—(CH₂)ₜ-aryl, —NH—(CH₂)ₜ-heteroaryl, —(CH₂)ₜ—COR⁵, —(CH₂)ₜ—SO₂R⁵, or —(CH₂)ₜ—CO₂R⁵; wherein said alkyl, alkenyl, —(CH₂)ₜ-cycloalkyl, —(CH₂)ₜ-heterocycloalkyl, ˜(CH₂)ₜ˜aryl, or ˜(CH₂)ₜ˜heteroaryl R^{1a} or R^{1b} substituent is optionally substituted with one to three hydroxyl, aryl, heteroaryl, halogen, alkyl, cycloalkyl, —SO₂R⁷, —NR⁷—COR⁷, —CON(R⁷)₂, —COOR⁷, —C(O)R⁷, —CN, or —N(R⁷)₂ wherein said aryl, alkyl, cycloalkyl and heteroaryl substituent is optionally substituted with one to three halogen, alkyl, hydroxyl, or ˜O˜alkyl; or R^{1a} and R^{1b} together with the carbon they are bonded to form a cycloalkylene moiety or a heterocycloalkylene moiety, wherein said cycloalkylene or heterocycloalkylene moiety is optionally substituted with one to three hydroxyl, aryl, heteroaryl, halogen, alkyl, cycloalkyl, —SO₂R⁷, —NR⁷—COR⁷, —CON(R⁷)₂, —COOR⁷, —C(O)R⁷, —CN, or —N(R⁷)₂, wherein said aryl, alkyl, cycloalkyl and heteroaryl substituent is optionally substituted with one to three halogen, alkyl, hydroxyl, or —O-alkyl;
when [symbol] is a double bond, R^{1b} is absent and R^{1a} is hydrogen, alkyl, alkenyl, ˜(CH₂)ₜ˜cycloalkyl, ˜(CH₂)ₜ˜heterocycloalkyl, ˜(CH₂)ₜ˜aryl, ˜(CH₂)ₜ˜heteroaryl, —(CH₂)ₜ—OR⁵, —(CH₂)ₜN(R⁷)₂, —NH—(CH₂)ₜ-cycloalkyl, —NH—(CH₂)ₜ-heterocycloalkyl, —NH—(CH₂)ₜ˜aryl, ˜NH˜(CH₂)ₜ˜heteroaryl, ˜(CH₂)ₜ˜COR⁵, ˜(CH₂)ₜ˜SO₂R⁵, or ˜(CH₂)ₜ˜CO₂R⁵, wherein said alkyl, alkenyl, ˜(CH₂)ₜ˜cycloalkyl, ˜(CH₂)ₜ˜heterocycloalkyl, ˜(CH₂)ₜ˜aryl, or —(CH₂)ₜ-heteroaryl R^{1a} substituent is optionally substituted with one to three hydroxyl, aryl, heteroaryl, halogen, alkyl, cycloalkyl, —SO₂R⁷, —NR⁷—COR⁷, —CON(R⁷)₂, —COOR⁷, —C(O)R⁷, —CN, or —N(R⁷)₂, wherein said aryl, alkyl, cycloalkyl and heteroaryl substituent is optionally substituted with one to three halogen, alkyl, hydroxyl, or —O-alkyl:
R² is alkyl, cycloalkyl, or alkenyl wherein said alkyl, cycloalkyl, or alkenyl is optionally substituted with one to three halogen, hydroxyl, or cyano;
each R³ is independently halogen, alkyl, cyano, hydroxyl, ˜O˜alkyl, ˜O˜cycloalkyl, ˜SO₂R⁷, ˜N(R⁷)₂, ˜COR⁷, ˜CON(R⁷)₂, ˜(CH₂)ₜ˜cycloalkyl, ˜(CH₂)ₜ˜heterocycloalkyl, —(CH₂)ₜ-aryl, or —(CH₂)ₜ-heteroaryl wherein said R³ alkyl, —(CH₂)ₜ-cycloalkyl, —(CH₂)ₜ-heterocycloalkyl, —(CH₂)ₜ-aryl, or —(CH₂)ₜ-heteroaryl is optionally substituted with one to three R⁴;
each R⁴ is independently alkyl, halogen, cyano, ˜SO₂NHR⁷, ˜CON(R⁷)₃, —N(R⁷)₂, —N(R⁷)COR⁷, —N(R⁷)CO₂R⁷, —SO₂N(R⁷)₂, —N(R⁷)SO₂R⁷, —COR⁷, —SO₂R⁷, —(CH₂)ₜ-cycloalkyl, —(CH₂)ₜ-heterocycloalkyl, —(CH₂)ₜ-aryl, —(CH₂)ₜ-heteroaryl, —(CH₂)ₜ—N(R⁷)₂, or —(CH₂)ₜ—OR⁵; wherein each R¹ alkyl, —(CH₂)ₜ-cycloalkyl, —(CH₂)ₜ-heterocycloalkyl, ˜(CH₂)ₜ˜aryl, or ˜(CH₂)ₜ˜heteroaryl is optionally independently substituted by one to three cyano, alkyl, halogen, ˜CF₃ or ˜OR⁵;
each R⁵ is independently hydrogen, alkyl, —(CH₂)ₜ-cycloalkyl, —(CH₂)ₜ-heterocycloalkyl, ˜(CH₂)ₜ˜aryl, or ˜(CH₂)ₜ˜heteroaryl: wherein said ˜(CH₂)ₜ˜cycloalkyl, ˜(CH₂)ₜ˜heterocycloalkyl, ˜(CH₂)ₜ˜aryl, or ˜(CH₂)ₜ˜heteroaryl is optionally substituted with one to three R⁶;
each R⁶ is independently alkyl, hydroxyl, alkoxy, halogen, cyano, ˜(CH₂)ₜN(R⁷)₂, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl;
each R⁷ is independently hydrogen, alkyl, —(CH₂)ₜ-cycloalkyl, —(CH₂)ₜ-heterocycloalkyl, —(CH₂)ₜ-aryl, or —(CH₂)ₜ-heteroaryl, or when two R⁷ substituents are attached to the same nitrogen atom they may be taken together with the nitrogen to which they are attached to form a heterocycloalkylene moiety; and wherein said alkyl, ˜(CH₂)ₜ˜cycloalkyl, ˜(CH₂)ₜ˜heterocycloalkyl, ˜(CH₂)ₜ˜aryl, or ˜(CH₂)ₜ˜heteroaryl are optionally substituted with one to three alkyl, halogen, cyano, hydroxyl, or —OR⁴;
n is an integer selected from 1, 2 and 3; and
each t is an integer independently selected from 0, 1, 2 and or pharmaceutically acceptable salts thereof.

2. A compound of claim 1 wherein A is aryl, heteroaryl, cycloalkyl or heterocycloalkyl, and A is optionally substituted with one R⁴ substituent.

3. A compound of claim 2 wherein A is aryl or heteroaryl, and R⁴ is independently alkyl, halogen, cyano, —SO₂NHR⁷, —CON(R⁷)₂, —N(R⁷)₂, —N(R⁷)COR⁷, —SO₂N(R⁷)₂, —N(R⁷)SO₂R⁷, —COR⁷, —SO₂R⁷, —(CH₂)ₜ-cycloalkyl, —(CH₂)ₜ-heterocycloalkyl, ˜(CH₂)ₜ˜aryl, ˜(CH₂)ₜ˜heteroaryl, ˜(CH₂)ₜ˜N(R⁷)₂, or ˜(CH₂)ₜ˜OR⁵ wherein each R⁴ alkyl, ˜(CH₂)ₜ˜cycloalkyl, ˜(CH₂)ₜ˜heterocycloalkyl, ˜(CH₂)˜aryl, or —(CH₂)ₜ-heteroaryl is optionally independently substituted by one to three cyano, alkyl, halogen, —CF₃, or —OR⁵.

4. A compound of claim 1 wherein A is aryl, heteroaryl, cycloalkyl or heterocycloalkyl, and A is optionally substituted with two R⁴ substituents.

5. A compound of claim 4 where in A is aryl or heteroaryl and each R⁴ is independently alkyl, halogen, cyano, —SO₂NHR⁷, —CON(R⁷)₂, —N(R⁷)₂—N(R⁷)COR⁷, —SO₂N(R⁷)₂, —N(R⁷)SO₂R⁷, —COR⁷, —SO₂R⁷, —(CH₂)ₜ-cycloalkyl, —(CH₂)ₜ-heterocycloalkyl, ˜(CH₂)ₜ˜aryl, ˜(CH₂)ₜ˜heteroaryl, ˜(CH₂)ₜ˜N(R⁷)₂, or ˜(CH₂)ₜ˜OR⁵ wherein each R⁴ alkyl, ˜(CH₂)ₜ˜cycloalkyl, ˜(CH₂)ₜ˜heterocycloalkyl, ˜(CH₂)ₜ˜aryl, or —(CH₂)ₜ-heteroaryl is optionally independently substituted by one to three cyano, alkyl, halogen, ˜CF₃, or ˜OR⁵.

6. A compound of claim 5 wherein at least one R⁴ is —(CH₂)ₜ-aryl wherein t is zero and the aryl is optionally substituted by one to three cyano, alkyl, halogen, or —OR⁵.

7. A compound of claim 5 wherein each R⁴ is —OR⁵.

8. A compound of claim 1 wherein A is aryl, heteroaryl, cycloalkyl or heterocycloalkyl, and A is optionally substituted with three R⁴ substituents.

9. A compound of claim 8 wherein A is aryl or heteroaryl and each R⁴ is independently alkyl, halogen, cyano, ˜SO₂NHR⁷, ˜CON(R⁷)₂, ˜N(R⁷)₂, ˜N(R⁷)COR⁷, —SO₂N(R⁷)₂, —N(R⁷)SO₂R⁷, —COR⁷, —(CH₂)ₜ-cycloalkyl, —(CH₂)ₜ-heterocycloalkyl, ˜(CH₂)ₜ˜aryl, ˜(CH₂)ₜ˜heteroaryl, ˜(CH₂)ₜ˜N(R⁷)₂, or ˜(CH₂)ₜ˜OR⁵ wherein each R⁴ alkyl, ˜(CH₂)ₜ˜cycloalkyl, ˜(CH₂)ₜ˜heterocycloalkyl, ˜(CH₂)ₜ˜aryl, or —(CH₂)ₜ-heteroaryl is optionally independently substituted by one to three cyano, alkyl, halogen, —CF₃, or —OR⁵.

10. A compound of claim 1 wherein B is aryl and is substituted with only one to three R³ substituents.

11. A compound of claim 10 wherein B is aryl and is substituted with only one R³ substituent wherein R³ is halogen.

12. A compound of claim 1 wherein [symbol] is a single bond, and R^{1a} and R^{1b} are each independently hydrogen or alkyl.

13. A compound of claim 12 wherein R^{1a} and R^{1b} together with the carbon they are bonded to form a cycloalkylene moiety or a heterocycloalkylene moiety.

14. A compound of claim 12 wherein R^{1a} and R^{1b} are each hydrogen.

15. A compound of claim 1 wherein [symbol] is a double bond, and R^{1b} is absent.

16. A compound of claim 1 wherein said compound of Formula (I) is a compound having Formula (II) 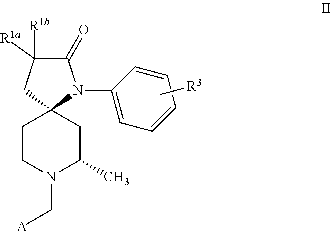
wherein A, R^{1a}, R^{1b}, and R³ are as defined in claim 1; or a pharmaceutically acceptable salt thereof.

17. A compound of claim 1 wherein said compound of Formula (I) is a compound having Formula (III) 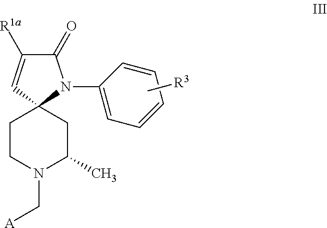
wherein A, R^{1a}, and R³ are as defined in claim 1; or a pharmaceutically acceptable salt thereof.

18. A method for the treatment of a disease or condition selected from the group consisting of neurological and psychiatric disorders comprising administering to the mammal an effective amount of compound of claim 1 or pharmaceutically acceptable salt thereof.

19. A pharmaceutical composition comprising a compound of claim 1 or a pharmaceutically acceptable salt thereof and a pharmaceutically acceptable carrier.

20. The composition of claim 17 further comprising an atypical antipsychotic, a cholinesterase inhibitor, dimebon or NMDA receptor antagonist.
